SEQUENCE LISTING (84 sequences)

SEQ ID NO: 1 - DNA, 16569 bp, Homo sapiens
  variation 3107: note = a, c, t, g, unknown or absent
  source 1..16569: mol_type = genomic DNA
gatcacaggtctatcaccctattaaccactcacgggagctctccatgcatttggtattttcgtctggggggtatgcacgcgatagcattgcgagacgctggagccggagcaccctatgtcgcagtatctgtctttgattcctgcctcatcctattatttatcgcacctacgttcaatattacaggcgaacatacttactaaagtgtgttaattaattaatgcttgtaggacataataataacaattgaatgtctgcacagccactttccacacagacatcataacaaaaaatttccaccaaaccccccctcccccgcttctggccacagcacttaaacacatctctgccaaaccccaaaaacaaagaaccctaacaccagcctaaccagatttcaaattttatcttttggcggtatgcacttttaacagtcaccccccaactaacacattattttcccctcccactcccatactactaatctcatcaatacaacccccgcccatcctacccagcacacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaaggaacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactaagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctacnttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgaaccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaactaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggtcataaagcctaaatagcccacacgttccccttaaataagacatcacgatg

SEQ ID NO: 2 - DNA, 783 bp, Homo sapiens
  misc_feature 81..82: note = Positions 81 and 82 comprises the joining position between mutated left gene and mutated right gene
  source 1..783: mol_type = genomic DNA
atggcccaccataattacccccatactccttacactattcctcatcacccaactaaaaatattaaacacaaactaccacctacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 3 - DNA, 565 bp, Homo sapiens
  misc_feature 407..408: note = Positions 407 and 408 comprises the joining position between mutated left gene and mutated right gene
  source 1..565: mol_type = genomic DNA
atggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcct

SEQ ID NO: 4 - DNA, 1905 bp, Homo sapiens
  misc_feature 665..666: note = Positions 665 and 666 comprises the joining position between mutated left gene and mutated right gene
  source 1..1905: mol_type = genomic DNA
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 5 - DNA, 1905 bp, Homo sapiens; identical to SEQ ID NO: 4
  misc_feature 662..663: note = Positions 662 and 663 comprises the joining position between mutated left gene and mutated right gene
  source 1..1905: mol_type = genomic DNA

SEQ ID NO: 6 - DNA, 1629 bp, Homo sapiens
  misc_feature 309..310: note = Positions 309 and 310 comprises the joining position between mutated left gene and mutated right gene
  source 1..1629: mol_type = genomic DNA
ataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 7 - DNA, 1692 bp, Homo sapiens
  misc_feature 357..358: note = Positions 357 and 358 comprises the joining position between mutated left gene and mutated right gene
  source 1..1692: mol_type = genomic DNA
atgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 8 - DNA, 572 bp, Homo sapiens
  misc_feature 387..388: note = Positions 387 and 388 comprises the joining position between mutated left gene and mutated right gene
  source 1..572: mol_type = genomic DNA
atggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaacaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataa

SEQ ID NO: 9 - DNA, 652 bp, Homo sapiens
  misc_feature 560..561: note = Positions 560 and 561 comprises the joining position between mutated left gene and mutated right gene
  source 1..652: mol_type = genomic DNA
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccactaatagttatgtcatccctcttattaatcatcatcctagccctaagtctggcctatgagtgactacaaaaaggattagactgaaccgaat

SEQ ID NO: 10 - DNA, 1453 bp, Homo sapiens
  misc_feature 553..554: note = Positions 553 and 554 comprises the joining position between mutated left gene and mutated right gene
  source 1..1453: mol_type = genomic DNA
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcct

SEQ ID NO: 11 - DNA, 1705 bp, Homo sapiens
  misc_feature 1357..1358: note = Positions 1357 and 1358 comprises the joining position between mutated left gene and mutated right gene
  source 1..1705: mol_type = genomic DNA
atgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcct

SEQ ID NO: 12 - DNA, 1361 bp, Homo sapiens
  misc_feature 66..67: note = Positions 66 and 67 comprises the joining position between mutated left gene and mutated right gene
  source 1..1361: mol_type = genomic DNA
atgccccaactaaatactaccgtatggcccaccataattacccccatactccttacactattcctcaccacccacagcctaattttagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatagactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctctt

SEQ ID NO: 13 - RNA, 783 nt, Homo sapiens
  source 1..783: mol_type = genomic RNA
atggcccaccataattacccccatactccttacactattcctcatcacccaactaaaaatattaaacacaaactaccacctacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 14 - RNA, 565 nt, Homo sapiens
  source 1..565: mol_type = genomic RNA
atggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcct

SEQ ID NO: 15 - RNA, 1905 nt, Homo sapiens
  source 1..1905: mol_type = genomic RNA
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 16 - RNA, 1905 nt, Homo sapiens; identical to SEQ ID NO: 15
  source 1..1905: mol_type = genomic RNA

SEQ ID NO: 17 - RNA, 1629 nt, Homo sapiens
  source 1..1629: mol_type = genomic RNA
ataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 18 - RNA, 1692 nt, Homo sapiens
  source 1..1692: mol_type = genomic RNA
atgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 19 - RNA, 572 nt, Homo sapiens
  source 1..572: mol_type = genomic RNA
atggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaacaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataa

SEQ ID NO: 20 - RNA, 652 nt, Homo sapiens
  source 1..652: mol_type = genomic RNA
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccactaatagttatgtcatccctcttattaatcatcatcctagccctaagtctggcctatgagtgactacaaaaaggattagactgaaccgaat

SEQ ID NO: 21 - RNA, 1453 nt, Homo sapiens
  source 1..1453: mol_type = genomic RNA
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcct

SEQ ID NO: 22 - RNA, 1705 nt, Homo sapiens
  source 1..1705: mol_type = genomic RNA
atgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcct

SEQ ID NO: 23 - RNA, 1362 nt, Homo sapiens
  source 1..1362: mol_type = genomic RNA
atgccccaactaaatactaccgtatggcccaccataattacccccatactccttacactattcctcaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatagactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctctt

SEQ ID NO: 24 - protein, 260 aa, Homo sapiens
MAHHNYPHTPYTIPHHPTKNIKHKLPPTSLTIGSLALAGMPFLTGFYSKDHIIETANMSYTNAWALSITLIATSLTSAYSTRMILLTLTGQPRFPTLTNINENNPTLLNPIKRLAAGSLFAGFLITNNISPASPFQTTIPLYLKLTALAVTFLGLLTALDLNYLTNKLKMKSPLCTFYFSNMLGFYPSITHRTIPYLGLLTSQNLPLLLLDLTWLEKLLPKTISQHQISTSIITSTQKGMIKLYFLSFFFPLILTLLLIT

SEQ ID NO: 25 - protein, 189 aa, Homo sapiens
  VARIANT 189: note = X can be any naturally occurring amino acid
MAHAAQVGLQDATSPIMEELITFHDHALMIIFLICFLVLYALFLTLTTKLTNTNISDAQEMETVWTILPAIILVLIALPSLRILYMTDEVNDPSLTIKSIGHQWYWTYEYTDYGGLIFNSYMLPPLFLEPGDLRLLAADLLILTWIGGQPVSYPFTIIGQVASVLYFTTILILMPTISLIENKMLKWAX

SEQ ID NO: 26 - protein, 634 aa, Homo sapiens
MNENLFASFIAPTILGLPAAVLIILFPPLLIPTSKYLINNRLITTQQWLIKLTSKQMMTMHNTKGRTWSLMLVSLIIFIATTNLLGLLPHSFTPTTQLSMNLAMAIPLWAGTVIMGFRSKIKNALAHFLPQGTPTPLIPMLVIIETISLLIQPMALAVRLTANITAGHLLMHLIGSATLAMSTINLPSTLIIFTILILLTILEIAVALIQAYVFTLLVSLYLHSNSWDPQQMALLNANPSLTPLLGLLLAAAGKSAQLGLHPWLPSAMEGPTPVSALLHSSTMVVAGIFLLIRFHPLAENSPLIQTLTLCLGAITTLFAAVCALTQNDIKKIVAFSTSSQLGLMMVTIGINQPHLAFLHICTHAFFKAMLFMCSGSIIHNLNNEQDIRKMGGLLKTMPLTSTSLTIGSLALAGMPFLTGFYSKDHIIETANMSYTNAWALSITLIATSLTSAYSTRMILLTLTGQPRFPTLTNINENNPTLLNPIKRLAAGSLFAGFLITNNISPASPFQTTIPLYLKLTALAVTFLGLLTALDLNYLTNKLKMKSPLCTFYFSNMLGFYPSITHRTIPYLGLLTSQNLPLLLLDLTWLEKLLPKTISQHQISTSIITSTQKGMIKLYFLSFFFPLILTLLLIT

SEQ ID NO: 27 - protein, 634 aa, Homo sapiens; identical to SEQ ID NO: 26

SEQ ID NO: 28 - protein, 542 aa, Homo sapiens
MNFALILMINTLLALLLMIITFWLPQLNGYMEKSTPYECGFDPMSPARVPFSMKFFLVAITFLLFDLEIALLLPLPWALQTTNLPLMVMSSLLLIIILALSLANTAAIQAILYNRIGDIGFILALAWFILHSNSWDPQQMALLNANPSLTPLLGLLLAAAGKSAQLGLHPWLPSAMEGPTPVSALLHSSTMVVAGIFLLIRFHPLAENSPLIQTLTLCLGAITTLFAAVCALTQNDIKKIVAFSTSSQLGLMMVTIGINQPHLAFLHICTHAFFKAMLFMCSGSIIHNLNNEQDIRKMGGLLKTMPLTSTSLTIGSLALAGMPFLTGFYSKDHIIETANMSYTNAWALSITLIATSLTSAYSTRMILLTLTGQPRFPTLTNINENNPTLLNPIKRLAAGSLFAGFLITNNISPASPFQTTIPLYLKLTALAVTFLGLLTALDLNYLTNKLKMKSPLCTFYFSNMLGFYPSITHRTIPYLGLLTSQNLPLLLLDLTWLEKLLPKTISQHQISTSIITSTQKGMIKLYFLSFFFPLILTLLLIT

SEQ ID NO: 29 - protein, 563 aa, Homo sapiens
MFADRWLFSTNHKDIGTLYLLFGAWAGVLGTALSLLIRAELGQPGNLLGNDHIYNVIVTAHAFVMIFFMVMPIMIGGFGNWLVPLMIGAPDMAFPRMNNMSFWLLPPSLLLLLASAMVEARADANTAAIQAILYNRIGDIGFILALAWFILHSNSWDPQQMALLNANPSLTPLLGLLLAAAGKSAQLGLHPWLPSAMEGPTPVSALLHSSTMVVAGIFLLIRFHPLAENSPLIQTLTLCLGAITTLFAAVCALTQNDIKKIVAFSTSSQLGLMMVTIGINQPHLAFLHICTHAFFKAMLFMCSGSIIHNLNNEQDIRKMGGLLKTMPLTSTSLTIGSLALAGMPFLTGFYSKDHIIETANMSYTNAWALSITLIATSLTSAYSTRMILLTLTGQPRFPTLTNINENNPTLLNPIKRLAAGSLFAGFLITNNISPASPFQTTIPLYLKLTALAVTFLGLLTALDLNYLTNKLKMKSPLCTFYFSNMLGFYPSITHRTIPYLGLLTSQNLPLLLLDLTWLEKLLPKTISQHQISTSIITSTQKGMIKLYFLSFFFPLILTLLLIT

SEQ ID NO: 30 - protein, 136 aa, Homo sapiens
MAHAAQVGLQDATSPIMEELITFHDHALMIIFLICFLVLYALFLTLTTKLTNTNISDAQEMETVWTILPAIILVLIALPSLRILYMTDEVNDPSLTIKSIGHQWYWTYEYTDYGGLIFNSYMLPPLFLEQATYSCT

SEQ ID NO: 31 - protein, 217 aa, Homo sapiens
MNENLFASFIAPTILGLPAAVLIILFPPLLIPTSKYLINNRLITTQQWLIKLTSKQMMTMHNTKGRTWSLMLVSLIIFIATTNLLGLLPHSFTPTTQLSMNLAMAIPLWAGTVIMGFRSKIKNALAHFLPQGTPTPLIPMLVIIETISLLIQPMALAVRLTANITAGHLLMHLIGSATLAMSTINLPLMVMSSLLLIIILALSLAYEWLQKGLDWTE

SEQ ID NO: 32 - protein, 484 aa, Homo sapiens
MNENLFASFIAPTILGLPAAVLIILFPPLLIPTSKYLINNRLITTQQWLIKLTSKQMMTMHNTKGRTWSLMLVSLIIFIATTNLLGLLPHSFTPTTQLSMNLAMAIPLWAGTVIMGFRSKIKNALAHFLPQGTPTPLIPMLVIIETISLLIQPMALAVRLTANITAGHLLMHLIGSATLAMSTINLHANGASMFFICLFLHIGRGLYYGSFLYSETWNIGIILLLATMATAFMGYVLPWGQMSFWGATVITNLLSAIPYIGTDLVQWIWGGYSVDSPTLTRFFTFHFILPFIIAALATLHLLFLHETGSNNPLGITSHSDKITFHPYYTIKDALGLLLFLLSLMTLTLFSPDLLGDPDNYTLANPLNTPPHIKPEWYFLFAYTILRSVPNKLGGVLALLLSILILAMIPILHMSKQQSMMFRPLSQSLYWLLAADLLILTWIGGQPVSYPFTIIGQVASVLYFTTILILMPTISLIENKMLKWA

SEQ ID NO: 33 - protein, 568 aa, Homo sapiens
MFADRWLFSTNHKDIGTLYLLFGAWAGVLGTALSLLIRAELGQPGNLLGNDHIYNVIVTAHAFVMIFFMVMPIMIGGFGNWLVPLMIGAPDMAFPRMNNMSFWLLPPSLLLLLASAMVEAGAGTGWTVYPPLAGNYSHPGASVDLTIFSLHLAGVSSILGAINFITTIINMKPPAMTQYQTPLFVWSVLITAVLLLLSLPVLAAGITMLLTDRNLNTTFFDPAGGGDPILYQHLFWFFGHPEVYILILPGFGMISHIVTYYSGKKEPFGYMGMVWAMMSIGFLGFIVWAHHMFTVGMDVDTRAYFTSATMIIAIPTGVKVFSWLATLHGSNMKWSAAVLWALGFIFLFTVGGLTGIVLANSSLDIVLHDTYYVVAHFHYVLSMGAVFAIMGGFIHWFPLFSGYTLDQTYAKIHFTIMFIGVNLTFFPQHFLGLSGMPRRYSDYPDAYTTWNIPPHIKPEWYFLFAYTILRSVPNKLGGVLALLLSILILAMIPILHMSKQQSMMFRPLSQSLYWLLAADLLILTWIGGQPVSYPFTIIGQVASVLYFTTILILMPTISLIENKMLKWA

SEQ ID NO: 34 - protein, 191 aa, Homo sapiens
MPQLNTTVWPTMITPMLLTLFLTTHSLIISIIPLLFFNQINNNLFSCSPTFSSDPLTTPLLMLTTWLLPLTIMASQRHLSSEPLSRKKLYLSMLISLQISLIMTFTATELIMFYIFFETTLIPTLAIITRWGNQPERLNAGTYFLFYTLVGSLPLLIALIYTHNTLGSLNILLLTLTAQELSNSWANNLMD

SEQ ID NO: 35 - DNA, 24 bp, synthetic construct
  misc_feature 1..24: note = Forward Primer
  source 1..24: mol_type = other DNA
tctaccccctctagagcccactgt

SEQ ID NO: 36 - DNA, 25 bp, synthetic construct
  misc_feature 1..25: note = Reverse Primer
  source 1..25: mol_type = other DNA
ctaggctgccaatggtgagggaggt

SEQ ID NO: 37 - DNA, 30 bp, synthetic construct
  misc_feature 1..30: note = Primer
  source 1..30: mol_type = other DNA
tgcgactcctagccgcagacctcctcattc

SEQ ID NO: 38 - DNA, 28 bp, synthetic construct
  misc_feature 1..28: note = Primer
  source 1..28: mol_type = other DNA
ggtacccaaatctgcttccccatgaaag

SEQ ID NO: 39 - DNA, 20 bp, synthetic construct
  misc_feature 1..20: note = Primer
  source 1..20: mol_type = other DNA
tgcgactcctagccgcagac

SEQ ID NO: 40 - DNA, 24 bp, synthetic construct
  misc_feature 1..24: note = Primer
  source 1..24: mol_type = other DNA
cgccatcatcctagtcctcatcgc

SEQ ID NO: 41 - DNA, 27 bp, synthetic construct
  misc_feature 1..27: note = Primer
  source 1..27: mol_type = other DNA
gaatgaggaggtctgcggctaggagtc

SEQ ID NO: 42 - DNA, 26 bp, synthetic construct
  misc_feature 1..26: note = Primer
  source 1..26: mol_type = other DNA
gtaagcctctacctacactccaactc

SEQ ID NO: 43 - DNA, 23 bp, synthetic construct
  misc_feature 1..23: note = Primer
  source 1..23: mol_type = other DNA
gcggatgagtaagaagattcctg

SEQ ID NO: 44 - DNA, 23 bp, synthetic construct
  misc_feature 1..23: note = Primer
  source 1..23: mol_type = other DNA
ggagacctaattgggctgatttg

SEQ ID NO: 45 - DNA, 20 bp, synthetic construct
  misc_feature 1..20: note = Primer
  source 1..20: mol_type = other DNA
ggccgtacgcctaaccgcta

SEQ ID NO: 46 - DNA, 29 bp, synthetic construct
  misc_feature 1..29: note = Primer
  source 1..29: mol_type = other DNA
gttgtgggtctcatgagttggagtgtagg

SEQ ID NO: 47 - DNA, 20 bp, synthetic construct
  misc_feature 1..20: note = Primer
  source 1..20: mol_type = other DNA
ccctggccgtacgcctaacc

SEQ ID NO: 48 - DNA, 33 bp, synthetic construct
  misc_feature 1..33: note = Primer
  source 1..33: mol_type = other DNA
atttgttgtgggtctcatgagttggagtgtagg

SEQ ID NO: 49 - DNA, 25 bp, synthetic construct
  misc_feature 1..25: note = Primer
  source 1..25: mol_type = other DNA
ccctaagtctggccaacacagcagc

SEQ ID NO: 50 - DNA, 22 bp, synthetic construct
  misc_feature 1..22: note = Primer
  source 1..22: mol_type = other DNA
gggtggagacctaattgggctg

SEQ ID NO: 51 - DNA, 25 bp, synthetic construct
  misc_feature 1..25: note = Primer
  source 1..25: mol_type = other DNA
acaacgttatcgtcacagcccatgc

SEQ ID NO: 52 - DNA, 27 bp, synthetic construct
  misc_feature 1..27: note = Primer
  source 1..27: mol_type = other DNA
gattgcttgaatggctgctgtgttggc

SEQ ID NO: 53 - DNA, 21 bp, synthetic construct
  misc_feature 1..21: note = Primer
  source 1..21: mol_type = other DNA
cgtctgaactatcctgcccgc

SEQ ID NO: 54 - DNA, 27 bp, synthetic construct
  misc_feature 1..27: note = Primer
  source 1..27: mol_type = other DNA
caattaggtgcatgagtaggtggcctg

SEQ ID NO: 55 - DNA, 20 bp, synthetic construct
  misc_feature 1..20: note = Primer
  source 1..20: mol_type = other DNA
aaggcacacctacacccctt

SEQ ID NO: 56 - DNA, 28 bp, synthetic construct
  misc_feature 1..28: note = Primer
  source 1..28: mol_type = other DNA
gagggatgacataactattagtggcagg

SEQ ID NO: 57 - DNA, 20 bp, synthetic construct
  misc_feature 1..20: note = Primer
  source 1..20: mol_type = other DNA
aaccaatagccctggccgta

SEQ ID NO: 58 - DNA, 31 bp, synthetic construct
  misc_feature 1..31: note = Primer
  source 1..31: mol_type = other DNA
gagggatgacataactattagtggcaggtta

SEQ ID NO: 59 - DNA, 21 bp, synthetic construct
  misc_feature 1..21: note = Primer
  source 1..21: mol_type = other DNA
ctatagcaccccctctacccc

SEQ ID NO: 60 - DNA, 27 bp, synthetic construct
  misc_feature 1..27: note = Primer
  source 1..27: mol_type = other DNA
gatgctaataattaggctgtgggtggt

SEQ ID NO: 61 - DNA, 20 bp, synthetic construct
  misc_feature 1..20: note = Primer
  source 1..20: mol_type = other DNA
acagtgaaatgccccaacta

SEQ ID NO: 62 - DNA, 20 bp, synthetic construct
  misc_feature 1..20: note = Primer
  source 1..20: mol_type = other DNA
gctcaggcgtttgtgtatga

SEQ ID NO: 63 - DNA, 20 bp, synthetic construct
  misc_feature 1..20: note = Primer
  source 1..20: mol_type = other DNA
caacgatccctcccttacca

SEQ ID NO: 64 - DNA, 21 bp, synthetic construct
  misc_feature 1..21: note = Primer
  source 1..21: mol_type = other DNA
agtacggatgctacttgtcca

SEQ ID NO: 65 - DNA, 20 bp, synthetic construct
  misc_feature 1..20: note = Primer
  source 1..20: mol_type = other DNA
gaagcgccaccctagcaata

SEQ ID NO: 66 - DNA, 20 bp, synthetic construct
  misc_feature 1..20: note = Primer
  source 1..20: mol_type = other DNA
ggtgaggcttggattagcgt

SEQ ID NO: 67 - DNA, 20 bp, synthetic construct
  misc_feature 1..20: note = Primer
  source 1..20: mol_type = other DNA
aatccaccccttacgagtgc

SEQ ID NO: 68 - DNA, 21 bp, synthetic construct
  misc_feature 1..21: note = Primer
  source 1..21: mol_type = other DNA
acaacgttatcgtcacagccc

SEQ ID NO: 69 - DNA, 20 bp, synthetic construct
  misc_feature 1..20: note = Primer
  source 1..20: mol_type = other DNA
gtgaggcttggattagcgtt

SEQ ID NO: 70 - DNA, 21 bp, synthetic construct
  misc_feature 1..21: note = Primer
  source 1..21: mol_type = other DNA
ctgaacctacgagtacaccga

SEQ ID NO: 71 - DNA, 21 bp, synthetic construct
  misc_feature 1..21: note = Primer
  source 1..21: mol_type = other DNA
gtgtgaaaacgtaggcttgga

SEQ ID NO: 72 - DNA, 21 bp, synthetic construct
  misc_feature 1..21: note = Primer
  source 1..21: mol_type = other DNA
tcgaaaccatcagcctactca

SEQ ID NO: 73 - DNA, 23 bp, synthetic construct
  misc_feature 1..23: note = Primer
  source 1..23: mol_type = other DNA
ccaattcggttcagtctaatcct

SEQ ID NO: 74 - DNA, 773 bp, Homo sapiens
  source 1..773: mol_type = genomic DNA
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 75 - DNA, 993 bp, Homo sapiens
  source 1..993: mol_type = genomic DNA
attaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctccatcatcacctcaacccaaaaaggcataattaaactttacttcctctctttcttcttcccactcatcctaaccctactcctaatcacataa

SEQ ID NO: 76 - RNA, 773 nt, Homo sapiens
  source 1..773: mol_type = genomic RNA
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 77 - RNA, 993 nt, Homo sapiens
  source 1..993: mol_type = genomic RNA
attaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctccatcatcacctcaacccaaaaaggcataattaaactttacttcctctctttcttcttcccactcatcctaaccctactcctaatcacataa

SEQ ID NO: 78 - protein, 222 aa, Homo sapiens
MSHQQPTNHHPTMTIKLTSKQMMTMHNTKGRTWSLMLVSLIIFIATTNLLGLLPHSFTPTTQLSMNLAMAIPLWAGTVIMGFRSKIKNALAHFLPQGTPTPLIPMLVIIETISLLIQPTALDLNYLTNKLKMKSPLCTFYFSNMLGFYPSITHRTIPYLGLLTSQNLPLLLLDLTWLEKLLPKTISQHQISTSIITSTQKGMIKLYFLSFFFPLILTLLLIT

SEQ ID NO: 79 - protein, 296 aa, Homo sapiens
MNMLAFIPVLTKKMNPRSTEAAIKYFLTQATASMILLMAILFNNMLSGQWTMTNTTNQYSSLMIMMAMAMKLGMAPFHFWVPEVTQGTPLTSGLLLLTWQKLAPISIMYQISPSLNVSLLLTLSILSIMAGSWGGLNQTQLRKILAYSSITHMGWMMAVLPYNPNMTILNLTIYIILTTTAFLLLNLNSSTTTLLLSRTWNKLTWLTPLIPSTLLSLGGLPPLTGFLPKWAIIEEFTKNNSLIIPTIMATITLLNLYFYLRLIYSTSIITSTQKGMIKLYFLSFFFPLILTLLLIT

SEQ ID NO: 80 - DNA, 21 bp, synthetic construct
  misc_feature 1..21: note = Forward Primer
  source 1..21: mol_type = other DNA
tgccctagcccacttcttacc

SEQ ID NO: 81 - DNA, 25 bp, synthetic construct
  misc_feature 1..25: note = Reverse (junction) Primer
  source 1..25: mol_type = other DNA
tagttgaggtctagggctgttggtt

SEQ ID NO: 82 - DNA, 25 bp, synthetic construct
  misc_feature 1..25: note = Forward Primer
  source 1..25: mol_type = other DNA
gggccattatcgaagaattcacaaa

SEQ ID NO: 83 - DNA, 24 bp, synthetic construct
  misc_feature 1..24: note = Reverse (junction) Primer
  source 1..24: mol_type = other DNA
gaggtgatgatggaggtggagtag

SEQ ID NO: 84 - protein, 259 aa, Homo sapiens
MAFMVKMPLYGLHLWLPKAHVEAPIAGSMVLAAVLLKLGGYGMMRLTLILNPLTKHMAYPFLVLSLWGMIMTSSICLRQTDLKSLIAYSSISHMALVVTAILIQTPWSFTGAVILMIAHGLTSSLLFCLANSNYERTHSRIMILSQGLQTLLPLMAFWWLLASLANLALPPTINLLGELSVLVTTFSWSNITLLLTGLNMLVTALYSLYMFTTTQWGSLTHHINNMKPSFTRENTLMFMHLSPILLLSLNPDIITGFSS

(19) United States
(12) Patent Application Publication
(10) Pub. No.: US 2026/0366080 A1
(43) Pub. Date: Nov. 19, 2026

(54) MITOCHONDRIAL DNA DELETIONS ASSOCIATED WITH ENDOMETRIOSIS

(71) Applicant: MDNA LIFE SCIENCES INC., Wilmington, DE (US)

(72) Inventors: Jennifer CREED, Kelowna (CA); Andrea MAGGRAH, Atikokan (CA); Brian REGULY, Chilliwack (CA); Andrew HARBOTTLE, Newcastle Upon Tyne (GB); Robert USHER, Houghton-Le-Spring (GB)

(21) Appl. No.: 19/259,639

(22) Filed: Jul. 3, 2025

Related U.S. Application Data
(63) Continuation of application No. 17/415,507, filed on Dec. 20, 2019, now abandoned.
(60) Provisional application No. 62/784,403, filed on Dec. 22, 2018.
(60) Provisional application No. 62/931,173, filed on Nov. 5, 2019.

(51) Int. Cl. C12Q 1/6883

(52) U.S. Cl. CPC C12Q 1/6883

(57) ABSTRACT

Aberrant mitochondrial DNA (mtDNA) molecules having specific large-scale deletions and having an association with endometriosis are provided. The aberrant, or mutated, mtDNA may comprise the parent nucleic acid (i.e. the large sublimon), particularly when re-circularized, wherein adjacent nucleotides are fused following the deletion to form a junction site. Alternatively, the mtDNA may comprise the deleted strand (i.e. the small sublimon), also particularly when re-circularized to create a junction site. In addition, fusion transcripts resulting from such mutated mtDNA, and their putative protein products, are provided, where such transcripts and proteins are also associated with endometriosis. Hybridization probes and amplification primers and kits containing same are provided for detecting, diagnosing, or monitoring endometriosis.

### CROSS REFERENCE TO RELATED APPLICATIONS

The present application is a continuation of U.S. application Ser. No. 17/415,507, filed Jun. 17, 2021 claiming priority to U.S. Application No. 62/784,403, filed Dec. 22, 2018, U.S. Application No. 62/931,173, filed Nov. 5, 2019, and PCT/US19/68098, filed Dec. 20, 2019. The entire contents of such prior applications are incorporated herein by reference.

### STATEMENT REGARDING SEQUENCE LISTING

The application contains a Sequence Listing which has been submitted electronically in .XML format and is hereby incorporated by reference in its entirety. Said .XML copy, created on Jul. 25, 2025, is named “066446_001CON.xml” and is 149,630 bytes in size. The sequence listing contained in this .XML file is part of the specification and is hereby incorporated by reference herein in its entirety.

### FIELD OF THE DESCRIPTION

The present description generally relates to novel biomarkers and methods for detecting/diagnosing and/or monitoring endometriosis. The description also relates to unique analytes and/or reagents that are useful in the subject methods.

### BACKGROUND

Endometriosis is a burdensome disease that occurs in up to 5% to 10% of women of reproductive age and is a common cause of infertility [1-7, 58]. The disease is characterized by the presence of endometrial tissue (epithelial cells and stroma) growing outside of the uterus. Such ectopic endometrial tissue can be found on the pelvic peritoneum and Fallopian tubes, the ovaries, the bowel and bladder, and rarely more distal body sites [8-11]. Women with endometriosis frequently suffer from often debilitating symptoms including non-menstrual pelvic pain, painful menstrual cramps, pain during intercourse, fatigue, and infertility [12], which can lead to a substantial reduction in quality of life [13]. Given its high prevalence and significant morbidity, endometriosis results in a very significant economic cost globally, estimated to be in the hundreds of billions of Euros each year [14].

Unfortunately, diagnosis of endometriosis is often a lengthy process, resulting a delay in treatment. The current “gold standard” for diagnosing endometriosis involves laparoscopic surgery followed by histopathological confirmation of tissue samples [5,15]. Making a timely diagnosis is further complicated by delayed reporting [16] and misinterpretation of symptoms [17] and can be even further delayed if the patient is hesitant to undergo a costly and invasive laparoscopic procedure. Indeed, the delay in diagnosing endometriosis can be more than a decade [16]. Due to these delays, a majority of women develop moderate to severe symptoms by the time a definitive diagnosis is made, which can result in increased morbidity, treatment costs, and decreased quality of life [14]. There is a therefore a need for a reliable, non-invasive test that can facilitate early detection of endometriosis and provide actionable real-time results. However, no non-invasive methods for detecting endometriosis are currently available.

Molecular biomarkers have been widely used as tools to measure, detect, and predict human disease [18-24]; however, the search for an endometriosis-specific biomarker has proven difficult [25]. Some of the key challenges include non-standardized sample collection, analysis methods, and data interpretation and the lack of biomarker specificity [17] though recent efforts have been made to harmonize methods of collecting and storage of biological specimen and reporting of endometriosis data, including the World Endometriosis Research Foundation (WERF) EPHect Protocols [26]. A variety of candidate biomarkers from blood, tissue, and urine have been reported, but none have been successfully translated into clinical use. Many of these candidates have specific limitations on sample collection such as biopsy from diseased tissue, requirement for collection during a particular phase of menstruation, or are dependent upon changes in regulatory patterns (e.g., gene expression, DNA methylation) induced by inflammation, which can overlap with other gynaecological disorders [10,17] and increase the likelihood of false positive detections. Thus, an ideal biomarker would be detectable from healthy cells or body fluids and independent of transient disease, inflammation-generated, or cyclical physiological changes.

The mitochondrial genome represents a less-explored biomarker repository. As shown in FIG. 1, the mitochondrial genome codes for a complement of 24 genes, including 2 rRNAs and 22 tRNAs that ensure correct translation of the remaining 13 genes which are vital to electron transport. Mitochondrial DNA (mtDNA) targets are attractive from a diagnostic perspective due to a high mutation frequency, limited DNA repair capability, presence in all nucleated cells, and high copy number (thousands of genomes per cell) [27]. As a result, even low frequency mutations or deletion events can be amplified reliably from heteroplasmic mitochondrial populations. Indeed, mtDNA mutations have been well-described as biomarkers for several cancers across multiple body sites including bone, brain, breast, lung, colorectal, gastric, ovarian, prostate, and endometrial tissues [28-37]. The mitochondrial (mt) genome is relatively small, having 16,569 nucleic acid base pairs, whereas the nuclear genome has over 3 billion base pairs. Furthermore, typically all mtDNA genomes in a given individual are identical given the clonal expansion of mitochondria within the ovum, once fertilization has occurred. The mt genome is also unusual in that it is a circular, intron-less DNA molecule interspersed with repeat motifs that flank specific lengths of sequences. Sequences between these repeats are prone to deletion under circumstances that are not well understood. Moreover, such deletions often include at least a portion of one or both of the flanking repeat sequences. As discussed further below, once the sequence constituting the deletion is removed, the remaining “parent” mtDNA re-circularizes to form a “large sublimon”. Similarly, the deleted sequence may also re-circularize to form a “small sublimon”. Given the number of repeats in the mt genome, there are many possible deletions. One of the best-known examples of these deletions is the 4977 bp “common deletion”, which has been associated with various disease states. Although the common deletion was also investigated as a marker for endometriosis [54], a lack of specificity did not suggest that such deletion would be an effective marker of the disease. Certain mitochondrial DNA deletions have been previous associated with some specific conditions and age-related disorders (see [59]-[64]). An 8686 bp deletion between nucleotides 5371-14058 of the mtDNA genome has also been published ([65]), but without any correlation with a disease state or condition.

In some cases, mtDNA deletions and other large-scale mtDNA rearrangements can result in a mutated mtDNA sequence that can be transcribed, resulting in a mitochondrial fusion transcript. Examples of associations between mitochondrial fusion transcripts and disease states have been described, for example, in the present Applicant's previous application numbers: PCT/CA2006/000652; PCT/CA2007/001711; PCT/CA2009/000351; and PCT/CA2010/000423, the entire disclosures of which are incorporated herein by reference.

MtDNA alterations have been detected in the endometrium during investigations of endometrial cancers [37-40]. However, these studies did not reveal a consensus region within the mtDNA genome or a specific mtDNA alteration that correlated to endometrial disease. As a result, these studies did not suggest a conclusion that mtDNA alternations could be used as a biomarker for detection of endometriosis. Further, no prior investigations are believed to have been conducted in relation to mitochondrial fusion transcripts and endometrial disease or state.

Thus, there exists a need for an accurate and/or more efficient means of detecting endometrial disease and/or condition that addresses at least one of the deficiencies in the known methods.

### SUMMARY OF THE DESCRIPTION

In one aspect, the present description provides methods, reagents, and/or kits for detecting, diagnosing, and/or monitoring endometriosis in a subject. The description involves the use of mitochondrial DNA (mtDNA) biomarkers, fusion transcripts thereof and/or translated fusion proteins that have been identified herein as being associated with endometriosis. The present methods can be conducted using a biological sample obtained from a subject being screened. Such sample may comprise tissue (such as a biopsy tissue), menstrual fluid, circulatory blood, or blood derivatives such as serum or plasma. The presently described methods can be performed on samples obtained non-invasively from subjects that are suspected of having or developing endometriosis and serve as an effective means of determining whether further invasive diagnostic investigation is necessary.

In one aspect, there is provided a method of detecting, diagnosing, and/or monitoring endometriosis in a mammalian subject, the method comprising identifying, in a biological sample from the subject, an aberrant mitochondrial DNA, mtDNA, molecule having at least one deletion resulting in a junction point in the rejoined, or re-circularized mtDNA nucleotide sequence, wherein the junction point is at nucleotide pairs 8469:13447, 7992:15730, 9191:12909, 9188:12906, 10367:12829, 6260:12814, 7973:9023, 9086:10313, 9079:14988, 7260:15540, 8431:10841, 8984:13833, or 5362:14049 of the mtDNA nucleotide sequence of SEQ ID NO: 1.

In one aspect, the method comprises identifying the aberrant mtDNA by contacting a biological sample with DNA probes or primers designed to hybridize to the aberrant mtDNA.

In one aspect, the method comprises identifying fusion transcripts of the aberrant mtDNA molecule(s).

In another aspect, the method comprises identifying fusion proteins encoded by the aberrant mtDNA molecule(s).

In one aspect, there is provided a method of identifying, in a biological sample from a mammalian subject, an aberrant mitochondrial DNA, mtDNA, molecule having a deletion, wherein the deletion comprises a nucleotide sequence between nucleotides 5362-14049; 8469-13447; 7992-15730; 9191-12909; 9188-12906; 10367-12829; 6260-12814; 7973-9023; 9086-10313; 9079-14988; 7260-15540; 8431-10841; or 8984-13833 of the mtDNA nucleotide sequence of SEQ ID NO: 1, and wherein, once re-circularized, the mtDNA includes a junction point.

In another aspect, there is provided a method of identifying, in a biological sample from a mammalian subject, an aberrant mitochondrial DNA, mtDNA, molecule having a deletion, wherein once re-circularized, the mtDNA includes a junction point consisting of first and second nucleotides, and wherein, with respect to SEQ ID NO: 1:
a) the deletion includes nucleotides 5377-14048, the first nucleotide is between nucleotides 5362-5377 and the second nucleotide is between nucleotides 14048-14063;
b) the deletion includes nucleotides 8483-13446, the first nucleotide is between nucleotides 8469-8483 and the second nucleotide is between nucleotides 13446-13460;
c) the deletion includes nucleotides 7993-15722, the first nucleotide is between nucleotides 7985-7993 and the second nucleotide is between nucleotides 15722-15730;
d) the deletion includes nucleotides 9196-12908, the first nucleotide is between nucleotides 9191-9196 and the second nucleotide is between nucleotides 12908-12912;
e) the deletion includes nucleotides 9196-12905, the first nucleotide is between nucleotides 9188-9196 and the second nucleotide is between nucleotides 12905-12913;
f) the deletion includes nucleotides 10368-12825, the first nucleotide is between nucleotides 10364-10368 and the second nucleotide is between nucleotides 12825-12829;
g) the deletion includes nucleotides 6261-12813, the first nucleotide is between nucleotides 6260-6271 and the second nucleotide is between nucleotides 12813-12824;
h) the deletion includes nucleotides 7984-9022, the first nucleotide is between nucleotides 7973-7984 and the second nucleotide is between nucleotides 9022-9033;
i) the deletion includes nucleotides 9087-10303, the first nucleotide is between nucleotides 9077-9087 and the second nucleotide is between nucleotides 10303-10313;
j) the deletion includes nucleotides 9086-14987, the first nucleotide is between nucleotides 9079-9086 and the second nucleotide is between nucleotides 14987-14904;
k) the deletion includes nucleotides 7261-15531, the first nucleotide is between nucleotides 7252-7261 and the second nucleotide is between nucleotides 15531-15540;
l) the deletion includes nucleotides 8440-10840, the first nucleotide is between nucleotides 8431-8440 and the second nucleotide is between nucleotides 10840-10849; or,
m) the deletion includes nucleotides 8994-13832, the first nucleotide is between nucleotides 8984-8994 and the second nucleotide is between nucleotides 13832-13842.

In another aspect, there are provided methods of detecting fusion transcripts and fusion proteins resulting from the aberrant mtDNA molecules or from the mtDNA deletions.

### BRIEF DESCRIPTION OF THE FIGURES

The features of certain embodiments will become more apparent in the following detailed description in which reference is made to the appended figures wherein:

FIG. 1 is an illustration showing mitochondrial coding genes.

FIGS. 2A to 2J illustrate the detection of fusion transcripts 1, 4, 14, 16, 120, 122, 193, 400, 516, and 586 in endometrial tissues as discussed in Example 1. Scatterplots represent normalized results of endometrial control tissues and endometriosis positive tissues that were tested against probes specific to ten fusion transcripts, identified as transcript numbers: 1 (FIG. 2A); 4 (FIG. 2B); 14 (FIG. 2C); 16 (FIG. 2D); 120 (FIG. 2E); 122 (FIG. 2F); 193 (FIG. 2G); 400 (FIG. 2H); 516 (FIG. 2I); and 586 (FIG. 2J). The y-axis of each figure indicates the normalized Relative Luminescence Units, RLUs, (log 2LOQProbe-Log2LOQHK23) where HK23 is the nuclear housekeeper transcript Human beta-2-microglobulin. The x-axis of each FIG. indicates tissue diagnosis, as determined by physician's diagnosis upon laparoscopic exam, where: endometrial control=0.0 and endometriosis positive=1.0.

FIG. 3 depicts an mtDNA fusion transcript map illustrating the mtDNA genome of SEQ ID NO: 1, gene locations and locations of the 10 mtDNA deleted portions described herein (i.e., “probes” or “targets”), which are indicated by a line spanning the length of each deletion.

FIGS. 4A and 4B illustrate the diagnostic accuracy of the 1.2 kb and 3.7 kb Deletions of Example 2, comparing symptomatic control samples and samples from patients with confirmed endometrial disease conditions. The 1.2 kb and 3.7 kb Deletions were evaluated for the ability to distinguish between symptomatic patient specimens and specimens from patients with confirmed endometriosis (all subtypes/stages combined). Receiver operator characteristic curves were constructed and the areas under the curves were calculated. Abbreviations: CI=confidence interval; ROC=receiver operator characteristic; Std=standard; vs=versus.

FIGS. 5A to 5D illustrate the diagnostic accuracy of the 1.2 kb Deletion of Example 2, in differentiating between symptomatic control samples and samples of different endometrial disease subtype. The 1.2 kb Deletion was evaluated for the ability to distinguish between symptomatic patient specimens and specimens from patients stratified by subtype of endometriosis (peritoneal, ovarian, deep infiltrating). FIG. 5A illustrates the distribution of normalized 1.2 kb Deletion for specimens from symptomatic controls and patients with peritoneal, ovarian or deep infiltrating endometriosis. Box boundaries represent the, 25^{th} and 75^{th} percentile, the line in the middle represents the median, and the whiskers represent the 90th (top) and 10th (bottom) percentiles. Dots represent outlier values (left). Descriptive statistics are summarized for each group (right). In FIGS. 5B to 5D receiver operator characteristic curves for the 1.2 kb Deletion were constructed and the areas under the curves were calculated, showing diagnostic accuracy. Abbreviations: CI=confidence interval; Dev=deviation; DIE=deep infiltrating endometriosis; N=number of specimens in each group; ROC=receiver operator characteristic; Std=standard; vs=versus.

FIGS. 6A to 6D illustrate the diagnostic accuracy of the 3.7 kb Deletion of Example 2, in differentiating between symptomatic control samples and samples of endometrial disease subtype. The 3.7 kb Deletion was evaluated for the ability to distinguish between symptomatic patient specimens and specimens from patients stratified by subtype of endometriosis (peritoneal, ovarian, deep infiltrating). FIG. 6A illustrates the distribution of normalized 3.7 kb Deletion for specimens from symptomatic controls and patients with peritoneal, ovarian or deep infiltrating endometriosis. Box boundaries represent the, 25th and 75th percentile, the line in the middle represents the median, and the whiskers represent the 90th (top) and 10th (bottom) percentiles. Dots represent outlier values (left). Descriptive statistics are summarized for each group (right). In FIGS. 6B to 6D receiver operator characteristic curves for the 3.7 kb deletion were constructed and the areas under the curves were calculated, showing diagnostic accuracy. Abbreviations: CI=confidence interval; Dev=deviation; DIE=deep infiltrating endometriosis; N=number of specimens in each group; ROC=receiver operator characteristic; Std=standard; vs=versus.

FIGS. 7A to 7C illustrate the diagnostic accuracy of the 1.2 kb Deletion of Example 2 in differentiating between symptomatic control samples and samples from patients with known disease stages. The 1.2 kb Deletion was evaluated for the ability to distinguish between symptomatic patient specimens and specimens from patients stratified by stage (low or high) of endometriosis. FIG. 7A illustrates the distribution of normalized 1.2 kb Deletion for specimens from symptomatic controls and patients with low (I/UU) or high (III/IV) stages of endometriosis. Box boundaries represent the, 25^{th} and 75^{th} percentile, the line in the middle represents the median, and the whiskers represent the 90th (top) and 10th (bottom) percentiles. Dots represent outlier values (left). Descriptive statistics are summarized for each group (right). In FIGS. 7B and 7C receiver operator characteristic curves for the 1.2 kb Deletion were constructed and the areas under the curves were calculated, showing diagnostic accuracy. Abbreviations: CI=confidence interval; Dev=deviation; N=number of specimens in each group; ROC=receiver operator characteristic; Std=standard; vs=versus.

FIGS. 8A to 8C illustrate the diagnostic accuracy of the 3.7 kb Deletion of Example 2 in differentiating between symptomatic control samples and samples from patients with known disease stages. The 3.7 kb Deletion was evaluated for the ability to distinguish between symptomatic patient specimens and specimens from patients stratified by stage (low or high) of endometriosis. FIG. 8A illustrates the distribution of normalized 3.7 kb Deletion for specimens from symptomatic controls and patients with low (I/II) or high (III/IV) stages of endometriosis. Box boundaries represent the, 25^{th} and 75^{th} percentile, the line in the middle represents the median, and the whiskers represent the 90th (top) and 10th (bottom) percentiles. Dots represent outlier values (left). Descriptive statistics are summarized for each group (right). In FIGS. 8B and 8C, receiver operator characteristic (ROC) curves for the 3.7 kb Deletion were constructed and the areas under the curves were calculated, showing diagnostic accuracy. Abbreviations: CI=confidence interval; Dev=deviation; N=number of specimens in each group; ROC=receiver operator characteristic; Std=standard; vs=versus.

FIG. 9 is a scatterplot showing the difference in the 8.7 kb deletion score between endometriosis positive samples, symptomatic controls samples and normal healthy control samples.

FIG. 10 is a box and whisker plot showing the difference in the 8.7 kb deletion score between endometriosis positive samples, symptomatic controls samples and normal healthy control samples.

FIG. 11 shows the ROC curves for the 8.7 kb deletion comparing endometriosis positive patients vs. healthy/normal controls.

FIG. 12 illustrates the diagnostic accuracy of the 8.7 kb deletion—symptomatic vs. all endometrial disease. The 8.7 kb deletion was evaluated for its ability to distinguish between samples from symptomatic patients and those from patients with confirmed endometriosis (all subtypes/stages combined) by calculating the area under ROC curves. Abbreviations: CI=confidence interval; ROC=receiver operator characteristic; Std=standard; vs=versus.

FIGS. 13A to 13B further illustrate the diagnostic accuracy of the 8.7 kb deletion-control vs. disease by subtype. These figures illustrate a study of whether the 8.7 kb deletion assay could distinguish between samples from symptomatic participants and those from participants stratified by endometriosis subtype (peritoneal, ovarian, deep infiltrating). FIG. 13A shows the normalized 8.7 kb deletion distribution for specimens from asymptomatic and symptomatic controls, participants with peritoneal, ovarian or deep infiltrating endometriosis. The box boundaries represent the 25th and 75th percentile, the line in the middle represents the median, and the whiskers represent the 90th (top) and 10th (bottom) percentiles. The dots represent outlier values (left). Descriptive statistics are also summarized for each group. FIGS. 13B to 13D show the areas under the ROC curves, which were calculated to show diagnostic accuracy. Abbreviations: As Con=asymptomatic controls; CI=confidence interval; Dev=deviation; DIE=deep infiltrating endometriosis; N=number of specimens in each group; ROC=receiver operator characteristic; Sym Con=symptomatic controls; Std=standard; vs=versus.

FIGS. 14A to 14C further illustrate the diagnostic accuracy of the 8.7 kb deletion-controls versus disease by stage. These figures illustrate whether the 8.7 kb deletion assay could distinguish between samples from symptomatic participants and those from participants stratified by endometriosis stages I/II and III/IV. FIG. 14A shows normalized 8.7 kb deletion distribution for specimens from symptomatic controls, participants with low (I/II) or high (III/IV) stages of endometriosis. The box boundaries represent the 25th and 75th percentile, the line in the middle represents the median, and the whiskers represent the 90th (top) and 10th (bottom) percentiles. The dots represent outlier values (left). Descriptive statistics are summarized for each group. FIGS. 14B and 14C show the areas under the ROC curves that were calculated to show diagnostic accuracy. Abbreviations: CI=confidence interval; Dev=deviation; N=number of specimens in each group; ROC=receiver operator characteristic; Sym Con=symptomatic controls; Std=standard; vs=versus.

FIG. 15 further illustrates the disease specificity of the 8.7 kb deletion for endometriosis. This figure summarizes the evaluation of the frequency of the 8.7 kb deletion in female cancers including endometrial cancer, ovarian cancer and breast cancer. Normalized 8.7 kb deletion distribution for specimens from endometrial cancer, ovarian cancer, breast cancer, symptomatic controls, and participants with peritoneal, ovarian or deep infiltrating endometriosis. The box boundaries represent the 25th and 75th percentile, the line in the middle represents the median, and the whiskers represent the 90th (top) and 10th (bottom) percentiles. The dots represent outlier values (left).

FIG. 16 is a scatterplot showing the difference in the 4.8 kb deletion score between endometriosis positive samples, symptomatic controls samples and normal healthy control samples.

FIG. 17 is a box and whisker plot showing the difference in the 4.8 kb deletion score between endometriosis positive samples, symptomatic controls samples and normal healthy control samples.

FIG. 18 illustrates a ROC for the 4.8 kb deletion comparing data from endometriosis positive patients vs. symptomatic controls.

FIG. 19 illustrates a ROC for the 4.8 kb deletion comparing data from endometriosis positive patients vs. healthy/normal controls.

FIG. 20 illustrates a deletion event according to the present description.

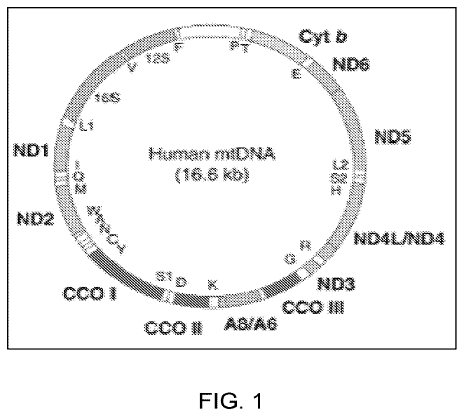

### DETAILED DESCRIPTION

Unless otherwise defined, all technical and scientific terms used herein have the same meaning as commonly understood by one of ordinary skill in the art to which this invention belongs. Although suitable materials and methods for the practice or testing of the present invention are described below, other known materials and methods similar or equivalent to those described herein can be used.

The terms “deletion”, “deletion fragment”, or “deletion sequence” as used herein with respect to mtDNA will be understood to mean a nucleotide sequence or segment that is removed, or deleted, from the wild-type or naturally occurring mtDNA genome.

The terms “wild-type mtDNA” or “naturally occurring mtDNA” refer to the Revised Cambridge Reference Sequence (rCRS) (2001, GenBank accession number: NC_012920.1), which is provided herein as SEQ ID NO: 1. Although this sequence is identified as being 16569 bp in length, the actual number of nucleotides is 16568. As known in the art, this sequence includes a gap or placeholder nucleotide at position 3107.

The term “mutation” or “aberration”, as used herein with respect to mtDNA, will be understood to be synonymous with the term “deletion”.

The term “mutated mtDNA” or “aberrant mtDNA”, as used in the context of the present description, will be understood as meaning a mtDNA molecule having at least one deletion (as defined above) in its genome sequence.

The terms “junction” or “junction point” will be understood to mean the location in the nucleotide sequence of the re-circularized mtDNA molecule that includes the re-joined, or spliced, nucleotides of the remaining mtDNA genome sequence following removal of the deletion. As discussed further herein, the deletion event typically results in the creation of two new sequence fragments, consisting of a parent sequence, corresponding to the rejoined mtDNA molecule after removal of the deletion, and deleted sequence, corresponding to the deleted section. Generally, the parent sequence is longer than the deleted sequence. Often, and as discussed above, both the long and short fragments re-circularize to form what are known, respectively, as the large and small sublimons. As would be understood, both of the sublimons would have a unique junction point in their nucleotide sequence. Thus, the terms junction or junction point may be used to refer to the either the large or small sublimons.

The phrase “having a deletion” will be understood to refer to a mtDNA molecule having a nucleotide sequence wherein a deletion sequence is removed. In other words, the phrase “a mtDNA having a deletion” refers to is the parent nucleic acid. Thus, a “mtDNA having the common deletion” means a mtDNA molecule having a sequence that does not include the 4977 bp deletion sequence.

As used herein, the term “detecting” will be understood to mean determining or identifying and/or measuring or quantifying the presence in a biological sample of a particular feature. In one aspect, the term “detecting” will be used herein to refer to the identification of a mitochondrial DNA (mtDNA) sequence, more particularly, a mtDNA having a deletion. The term “detecting” may also be used to refer to the identification of a mitochondrial fusion transcript and/or a protein encoded by such mtDNA molecule. In the latter instance, the protein is referred to herein as a “fusion protein” and would include an amino acid sequence that results from the translation of the rejoined mtDNA following a deletion event. Such mtDNA may comprise the parent, or aberrant mtDNA or the deleted sequence.

As used herein, the term “diagnosing” will be understood to mean the identification of a disease condition or disease state or the determination of a higher, or increased probability of the existence of a disease condition or disease state. For example, with respect to the present description, a higher probability of the existence of a state or condition of endometriosis will be deemed to exist, or “diagnosed” when a mtDNA molecule or fusion transcript described herein is detected. It will be understood that the actual or clinical diagnosis of the state or condition will be made by a clinician upon examination of a biopsy sample or other such means. Thus, in some cases, the terms “detecting” and “diagnosing” may be used interchangeably herein.

As used herein, the term “biological sample” will be understood to refer to a tissue or bodily fluid containing cells or nucleic acids from which a molecule of interest can be obtained. The biological sample can be used either directly as obtained from the source or be initially subjected to a pre-treatment to modify the character of the sample. In one aspect, the biological sample is blood, in particular circulatory blood, it being understood that the term “blood” as used herein is intended to include blood derivatives, such as plasma and/or serum. In another aspect, the biological sample is menstrual fluid including menstrual blood. In another aspect, the biological sample is a tissue sample obtained from a subject. In one aspect, circulatory blood may used as the biological sample. It will be understood that blood samples for the purpose of the present description may be drawn from any source on a subject's body. This would include, without limitation, blood drawn from venous sources by syringe etc., collection of menstrual fluid samples, or capillary blood, such as blood drawn by finger pricks. Employing the presently described methods using circulatory blood (including, as noted above, blood derivatives), provides an effective means of detecting the presence of endometriosis in an individual suspected of having such condition without having to unnecessarily undergo painful and risky invasive procedures. As noted above, in situations where the presently described methods indicate the presence of endometriosis, a diagnosis will still require a clinical assessment, and perhaps laparoscopic examination/surgery or analysis of a biopsy sample. Thus, it will be understood that, in one aspect, the presently described methods, in particular when using circulatory blood (or one or more derivatives thereof, as described above) as the biological sample, could be conducted on a sub-population of patients, including those individuals that have one or more indications suggestive of the presence of endometriosis. It will also be understood that the presently described methods may be conducted on general population members as an initial phase of screening endometriosis. In other words, the presently described methods may be performed on non-symptomatic subjects (i.e. individuals who do not present with symptoms).

As used herein, the phrase “mitochondrial fusion transcript” or “fusion transcript” refers to an RNA transcription product produced as a result of the transcription of a mtDNA sequence.

As used herein, the term “variant” refers to a nucleic acid sequence differing from a naturally occurring sequence but retaining the essential or functional properties thereof. In one aspect, the term “variant” may refer to a sequence that varies with respect to the wild-type sequence. Generally, in the case of mtDNA, variants are overall closely similar, and, in many regions, identical to a select mtDNA sequence. In the context of the present description, variants may comprise at least one of the nucleotides of the junction point of the spliced genes and may further comprise one or more nucleotides adjacent thereto. In one aspect, a variant sequence is at least 80%, 85%, 86%, 87%, 88%, 89%, 90%, 91%, 92%, 93%, 94%, 95%, 96%, 97%, 98% or 99% identical to a given mtDNA sequence described herein, or its complementary strand.

The phrase “substantially similar” as used herein refers to nucleic acids that are functionally the same but differing in their respective nucleic acid sequences. In one aspect, two sequences that are substantially similar to each other may be referred to as “variants”. Thus, two nucleic acid molecules may be considered substantially similar where a difference in one or more nucleotides between the respective nucleic sequences does not alter their functional properties or the functional properties of any polypeptides encoded by such nucleic acids. As would be understood, owing to the degeneracy of the genetic code, a base pair change can result in no change in the encoded amino acid sequence.

The phrase “substantial complementarity” refers to a sufficiently high degree of complementarity between the nucleotide sequences of nucleic acid molecules that allows hybridization there-between, but not necessarily 100% complementarity. For example, a primer or probe with substantial complementarity to a target sequence may have 80% to 99% sequence identity to the target sequence. In one aspect, substantial complementarity as used herein refers to at least 80%, 85%, 86%, 87%, 88%, 89%, 90%, 91%, 92%, 93%, 94%, 95%, 96%, 97%, 98% or 99% sequence identity between sequences.

The term “fragment” as used herein refers to a nucleic acid sequence that is a portion of a given mitochondrial genomic sequence, or the complementary strand thereto. In one aspect, such “portion” includes at least two of the nucleotides comprising the junction point of spliced genes and may further comprise one or more nucleotides adjacent thereto. That is, the portion comprises the rejoined, or re-circularized DNA sequence after removal of a deletion. The fragments described herein are at least about 150 nucleotides (nt) in length, at least about 75 nt, at least about 50 nt, at least about 40 nt, at least about 30 nt, at least about 20 nt, or preferably at least about 15 nt in length. Although certain minimum nucleotide lengths are recited above, it will be understood, as described herein, that fragments of any size (e.g., 50, 150, 200, 300, 400, 500, 600, 700, 800, 900, 1000, 1500, 2000, 2500, 3000, 4000, 5000, 6000, 7000, 8000 or more nucleotides) are also contemplated.

In the context of sequence lengths, the term “about” as used herein, includes the particularly recited value or a value larger or smaller by several (5, 4, 3, 2, or 1) nucleotides, at either terminus or at both termini.

As used herein, the term “probe” or “primer” refers to an oligonucleotide molecule that forms a duplex structure with, or “hybridizes” to, a target nucleic acid, due to complementarity of at least a portion of the nucleotide sequence of the probe/primer with a portion of the nucleotide sequence of the target molecule. The target nucleic acid molecule may in some cases be a fragment of a naturally occurring nucleic acid molecule. Probes described herein may be labeled according to methods known in the art. It will be understood that the probes or primers described herein would be used under suitable hybridizing conditions as would be known to persons skilled in the art. The probes herein may also be referred to hybridizing probes. The probes and primers described herein may be of any length, as would be understood by persons skilled in the art. By way of example only, the presently described probes and primers may have lengths of about 150, 140, 130, 120, 100, 90, 80, 70, 60, 50, 40, 30, 25, 20, 15, or 10 nucleotides (nt). In one preferred aspect, the probes and/or primers described herein are about 12 to about 35 nt in length, or preferably about 18 to about 25 nt in length, and more preferably about 15 nt in length. As would be appreciated by persons skilled in the art, probes may have longer nucleotide lengths than primers. Thus, in some cases, the probes described herein may have lengths of about 50, 100, 150, 200, 250, 300, 350, 400, 450, 500, 550, 600, 650, 700, 750, 800, 850, 900, 950, 1000, 1500, 2000, or 2500 nucleotides. The present description is not limited to any particular probe or primer length.

The terms “comprise”, “comprises”, “comprised” or “comprising” may be used in the present description. As used herein (including the specification and/or the claims), these terms are to be interpreted as specifying the presence of the stated features, integers, steps or components, but not as precluding the presence of one or more other feature, integer, step, component or a group thereof as would be apparent to persons having ordinary skill in the relevant art. Thus, the term “comprising” as used in this specification means “consisting at least in part of. When interpreting statements in this specification that include that term, the features, prefaced by that term in each statement, all need to be present but other features can also be present. Related terms such as “comprise” and “comprised” are to be interpreted in the same manner.

The term “and/or” can mean “and” or “or”.

Unless stated otherwise herein, the article “a” when used to identify any element is not intended to constitute a limitation of just one and will, instead, be understood to mean “at least one” or “one or more”.

As described herein, the present inventors have identified novel mtDNA deletions that are, in one aspect, associated with endometriosis and therefore constitute accurate diagnostic markers for such condition. The inventors have also identified novel mtDNA fusion transcripts that are, in one aspect, associated with endometriosis. Both of these aspects are discussed further below. Translation products resulting from the fusion transcripts are also encompassed by the present description.

In one aspect, the present description relates to the inventors' hypothesis that endometrial cells shed in menstrual fluid during menorrhea would harbour the same genetic profile as endometrial-like cells present in ectopic and/or eutopic endometrial lesions. Using the knowledge gained from mapping the large-scale deletions of the human mitochondrial genome, the observation of high frequencies of these deletions, and the evidence in other disease types of transcriptionally active mutated mtDNA molecules, the inventors further hypothesized that mitochondrial deletions and fusion transcripts might be present in endometrial-like cells present in ectopic and/or eutopic endometrial lesions.

To test these hypotheses, 268 mitochondrial fusion transcripts were selected, based on predicted direct and indirect repeats throughout the mitochondrial genome, and screened for their use as biomarkers of endometriosis. A number of mtDNA deletions and corresponding fusion transcripts were identified by the inventors as being particularly useful in distinguishing samples with endometriosis from those without endometriosis. These deletions and fusion transcripts are discussed further below. These mtDNA molecules produce fusion sequences having open reading frames (ORFs) that can be transcribed by mitochondrial transcription machinery, resulting in fusion transcripts. Protein products, or fusion proteins, encoded by such fusion transcripts are also expected to be produced.

1.0) mtDNA Deletions, Fusion Transcripts, and Translation Products
1.1) Mitochondrial DNA (mtDNA) Mutations

As discussed above, mtDNA mutations generally comprise a deletion of a portion of the mtDNA wild-type sequence. The present description is based on associations between specific mtDNA mutations, in particular deletions of the mtDNA genomic sequence, and endometriosis.

According to the present description, to determine candidate genomic sequences, junction points resulting from sequence deletions were first identified. Sequence deletions were primarily identified by direct or indirect repetitive elements which flank the sequence to be deleted at the 5′ and 3 end. The removal of a section of the nucleotides from the genome followed by the ligation of the remaining genome results in the creation of a novel junction point.

Upon identification of a junction point, the nucleotides of the genes flanking the junction point were determined in order to identify a spliced gene. Typically, the spliced gene comprises the initiation codon from the first gene and the termination codon of the second gene, and may be expressed as a continuous transcript, i.e. one that keeps the reading frame from the beginning to the end of both spliced genes. It is also possible that alternate initiation or termination codons contained within the gene sequences may be used.

Large-scale deletions in the mitochondrial genome often result in two products arising from the mutation process. These products are the result of the re-circularization of both parts of the mtDNA genome: 1) a short sequence that may, in one aspect, correspond to the deleted mtDNA sequence; and, 2) a long sequence that may, in one aspect, correspond to the remaining mtDNA genomic sequence. It will be understood that depending on the size of the deletion, the deletion may be larger than the remaining mtDNA. This situation would occur, for example, when the deleted sequence is larger than about 8200 bp in length. Often, both the short and long sequences re-circularize to form what are known, respectively, as the small and large sublimons. In cases where the small component is of an insufficient number of nucleotides, re-circularization is not possible, in which case the mutation process only results in a large sublimon. As discussed herein, both large and small sublimons can be identified thereby allowing both molecules to be used for detecting, diagnosing, and/or monitoring endometriosis.

### 1.2) Fusion Transcripts

Large-scale rearrangement mutations in the mitochondrial genome result in the generation of fusion transcripts. Thus, it was expected that mtDNA rearrangements associated with endometriosis would result in fusion transcripts that are also associated with endometriosis. Thus, the use of mtDNA encoding such transcripts and probes directed thereto for the diagnosis and monitoring of endometriosis are provided herein.

The present description provides the identification of fusion transcripts and associated hybridization probes and primers useful in methods for predicting, diagnosing, and/or monitoring endometriosis. One of skill in the art will appreciate that such molecules may be derived through the isolation of naturally-occurring transcripts or, alternatively, by the recombinant expression of mtDNA molecules isolated according to the methods of the invention. As discussed, such mtDNA molecules typically comprise a spliced gene having the initiation codon from the first gene and the termination codon of the second gene. Accordingly, fusion transcripts derived therefrom comprise a junction points associated with the spliced genes.

### 1.3) Translation Products

Based on the fusion transcripts described herein, the present description also provides amino acid sequences of putative proteins, i.e. “fusion proteins”, resulting from the translation of the subject fusion transcripts. The description also provides translation products of at least a portion of the fusion transcripts, in particular the portion comprising the transcribed fusion site, or junction point of the mtDNA.

Fusion proteins of the description can be recovered and purified from a biological sample by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, hydrophobic charge interaction chromatography and lectin chromatography. Most preferably, high performance liquid chromatography (“HPLC”) is employed for purification.

Assaying fusion protein levels in a biological sample can occur using a variety of techniques. For example, protein expression in tissues can be studied with classical immunohistological methods (Jalkanen et al., J. Cell. Biol. 101:976-985 (1985); Jalkanen, M., et al., J. Cell. Biol. 105:3087-3096 (1987)). Other methods useful for detecting protein expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (<125> I, <121> I), carbon (<14> C), sulfur (<35> S), tritium (<3> H), indium (<112> In), and technetium (<99m> Tc), and fluorescent labels, such as fluorescein and rhodamine, and biotin.

The polypeptides of the description can also be produced by recombinant techniques known in the art. Typically this involves transformation (including transfection, transduction, or infection) of a suitable host cell with an expression vector comprising a polynucleotide encoding the protein or polypeptide of interest.

### Antibodies and Protein Binding Agents

Protein specific antibodies for use in the assays of the present description can be raised against the wild-type or expressed fusion proteins described herein or an antigenic polypeptide fragment thereof, which may be presented together with a carrier protein, such as an albumin, to an animal system (such as rabbit or mouse) or, if it is long enough (at least about 25 amino acids), without a carrier. It will be understood that although antibodies are described, any other suitable binding agent, specific to identifying proteins, may also be used. In either case, the antibodies, or binding agents, are capable of identifying the fusion proteins described herein by means of specifically binding to a region of such proteins that is representative, or indicative, of the deletion. In one aspect, the fusions proteins have a unique amino acid profile that represents the translation of the junction point of the mtDNA molecule (either the large or small sublimon) after a deletion event.

As used herein, the term “antibody” (Ab) or “monoclonal antibody” (Mab) is meant to include intact molecules as well as antibody fragments, or antigen-binding fragments, thereof (such as, for example, Fab and F(ab′)2 fragments) which are capable of specifically binding to, or having “specificity to”, a mitochondrial fusion protein. Fab and F(ab′)2 fragments lack the Fc fragment of intact antibody, clear more rapidly from the circulation, and may have less non-specific tissue binding of an intact antibody (Wahl et al., J. Nucl. Med. 24:316-325 (1983)). Thus, these fragments are preferred.

The antibodies of the present invention may be prepared by any of a variety of methods. For example, cells expressing the mitochondrial fusion protein or an antigenic fragment thereof can be administered to an animal in order to induce the production of sera containing polyclonal antibodies. In one method, a preparation of mitochondrial fusion protein is prepared and purified to render it substantially free of natural contaminants. Such a preparation is then introduced into an animal in order to produce polyclonal antisera of greater specific activity.

In a related method, the antibodies of the present description are monoclonal antibodies. Such monoclonal antibodies can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., (1981) pp. 563-681). In general, such procedures involve immunizing an animal (preferably a mouse) with a mitochondrial fusion protein antigen or with a mitochondrial fusion protein-expressing cell.

In one aspect, the present description comprises immunological assays using antibodies or antigen-binding fragments having specificity to the fusion proteins described herein (as described above). Such immunological assays may be facilitated by kits containing the antibodies or antigen-binding fragments along with any other necessary reagents, test strips, materials, instructions etc.

### Assays

Measuring the level of a translation product such as a fusion protein in a biological sample can determine the presence or progression of endometriosis in a subject. Therefore, in one aspect, the present description provides methods for predicting, diagnosing or monitoring endometriosis, comprising obtaining one or more biological samples, extracting mitochondrial fusion proteins from the samples, and assaying the samples for such molecules by: quantifying the amount of one or more molecules in the sample and comparing the quantity detected with a reference value. As would be understood by those of skill in the art, the reference value is based on whether the method seeks to predict, diagnose or monitor endometriosis. Accordingly, the reference value may relate to protein data collected from one or more control sample, or biological samples not positive for endometriosis, from one or more biological samples positive for endometriosis, and/or from one or more biological samples taken over time.

Techniques for quantifying proteins in a sample are well known in the art and include, for instance, classical immunohistological methods (Jalkanen et al., J. Cell. Biol. 101:976-985 (1985); Jalkanen, M., et al., J. Cell. Biol. 105:3087-3096 (1987)). Additional methods useful for detecting protein expression include immunoassays such as the radioimmunoassay (RIA) and the enzyme linked immunosorbent assay (ELISA).

In one aspect, the description provides a method of detecting, diagnosing or monitoring endometriosis in a mammal, the method comprising assaying a tissue sample from the mammal for the presence of at least one mitochondrial fusion protein.

### 2.0) Probes and Primers

2.1) mtDNA Probes and Primers

Also described herein are mtDNA hybridization probes and/or primers capable of hybridizing to aberrant mtDNA sequences under suitable hybridizing conditions. Any known method of hybridization may be used.

Probes and/or primers may be generated directly against exemplary mtDNA fusion molecules described herein (such as those listed in Table 1 below), or to a fragment or variant thereof. For instance, the aberrant mtDNA sequences discussed herein can be used to design primers or probes that will detect a nucleic acid sequence comprising a fusion nucleotide sequence of interest. As would be understood by those of skill in the art, primers and/or probes that hybridize to these nucleic acid molecules may do so under highly stringent hybridization conditions or lower stringency conditions. Such conditions would be known to those skilled in the art and are described, for example, in Current Protocols in Molecular Biology (John Wiley & Sons, New York (1989)), 6.3.1-6.3.6.

In some aspects, the probes and primers described herein contain a sequence complementary to at least a portion of the aberrant mtDNA comprising the junction point of the spliced genes. As discussed above, this “portion” includes at least the two nucleotides remaining in the mtDNA genome after removal of the deletion, thereby resulting in a junction point, identified herein as A:B, where “A” and “B” represent the mtDNA genomic nucleotides on opposite sides of the deleted sequence, but which are adjacent to each other after the remaining sequence is re-circularized. The “portion” may further comprise one or more nucleotides adjacent to the junction point. In this regard, the present description encompasses any suitable targeting mechanism that will select a mtDNA molecule using the nucleotides involved in and/or adjacent to the junction point A:B. It is further contemplated herein that primer and probe sequences could be altered by one or more base pairs while still enabling hybridization to the target sequence. Such primers or probes will be referred to as having “substantial complementarity” to the target sequence. As discussed above, after a deletion event, both large and small sublimons may result, both of such sublimons would have a respective junction point, such as defined above, once the molecules are re-circularized.

Further, the present description encompasses primers that are designed, in one aspect, to span the deletion junction or junction point A:B in the forward or reverse direction. In another aspect, one or more primer may be designed to hybridize to a location on the target sequence that is adjacent the junction point.

Various types of probes known in the art are contemplated for use in the present description. For example, the probe may be a hybridization probe, the binding of which to a target nucleotide sequence can be detected using a general DNA binding dye such as ethidium bromide, SYBR® Green, SYBR® Gold and the like. Alternatively, the probe can incorporate one or more detectable labels. Detectable labels are molecules or that can be detected directly or indirectly and are chosen such that the ability of the probe to hybridize with its target sequence is not affected. Methods of labelling nucleic acid sequences are well-known in the art (see, for example, Ausubel et al., (1997 & updates) Current Protocols in Molecular Biology, Wiley & Sons, New York).

Labels suitable for use with the probes of the present description include those that can be directly detected, such as radioisotopes, fluorophores, chemiluminophores, enzymes, colloidal particles, fluorescent microparticles, and the like. One skilled in the art will understand that directly detectable labels may require additional components, such as substrates, triggering reagents, light, and the like to enable detection of the label. The present description also contemplates the use of labels that are detected indirectly.

As discussed above, the presently described probes and primers may be of any suitable length as would be understood by persons skilled in the art. Nucleotide lengths of the probes and primers of the present description were discussed above. As discussed above, the probes and/or primers described herein may preferably be about 12 to about 25 nucleotides in length, more preferably about 12 to about 15 nt in length. It will be understood that the primers and/or probes described herein may preferably be of a length that is at least the size of the mtDNA repeat (i.e. repeated) sequence. The present description is not limited to any particular primer or probe length.

The probes described herein will preferably hybridize to nucleic acid molecules from the biological samples described herein, thereby enabling the described methods. Accordingly, in one aspect, there is provided a hybridization probe for use in the detection of endometriosis, wherein the probe is complementary to, or substantially complementary to, at least a portion of an aberrant mtDNA molecule described herein or a portion of a deleted sequence from the mtDNA genome.

### 2.2) Fusion Transcript Probes and Primers

Once a fusion transcript has been characterized, primers or probes can be developed to target the transcript in a biological sample. Such primers and probes may be prepared using any known method (as described above) or as set out in the examples provided below. A probe may, for example, be generated for a fusion transcript, and detection technologies, such as QuantiGene™ 2.0 by Panomics™, can be used to detect the presence of the transcript in a sample. Primers and probes may be generated directly against exemplary fusion transcripts described herein, or to a fragment or variant thereof. For instance, the sequences set forth herein (such as those listed in Table 2 below) can be used to design probes or primers that will detect an RNA sequence comprising a fusion sequence of interest.

As would be understood by those skilled in the art, probes and primers designed to hybridize to the fusion transcripts described herein comprise sequences complementary, or substantial complementary, to at least a portion of the transcript expressing the junction point of the spliced genes. This portion includes at least two of the nucleotides complementary to the expressed junction point and may further comprise one or more complementary nucleotides adjacent thereto. In this regard, the present description encompasses any suitable targeting mechanism that will select a fusion transcript that uses the nucleotides involved and adjacent to the junction point of the spliced genes.

Various types of probes and methods of labelling known in the art are contemplated for the preparation of transcript probes described herein. Some examples of such types and methods have been described above with respect to the detection of genomic sequences. The transcript probes of the present description are at least about 150 nt, at least about 75 nt, at least about 50 nt, at least about 40 nt, at least about 30 nt, at least about 20 nt, or preferably at least about 12-15 nt in length. A probe of “at least 20 nt in length,” for example, is intended to include 20 or more contiguous bases that are complementary to an mtDNA sequence of the invention. Of course, larger probes (e.g., 50, 150, 500, 600, 2000 nucleotides) may be preferable. As mentioned above, primers or probes of 18 to 25 nt are preferable.

In some aspects, there is provided one or more hybridization probes and/or primers for use in the detection of endometriosis, wherein the one or more probes and/or primers is/are complementary to, or substantially complementary to, at least a portion of a mitochondrial fusion transcript described herein.

3.0) Assays for Detecting mtDNA Deletions Fusion Transcripts and Protein Products Thereof

As mentioned above, the present description provides mitochondrial DNA biomarkers that are useful in detecting, diagnosing, and/or monitoring endometriosis in a subject using a biological sample from the subject. In particular, such biological sample is non-invasively collected menstrual fluid, circulatory blood, and/or tissue (such as biopsy tissue). The description therefore provides, in one aspect, a menstrual-fluid- or blood-based test that will enable the early and accurate detection of endometriosis, thereby preventing unnecessary initial and repeat surgical procedures. Thus, the methods described herein will reduce the need for unnecessary laparoscopic procedures when endometriosis is suspected but not detected. The present methods will also aid in determining whether endometriosis has recurred by allowing for the monitoring of endometriosis in a subject over time.

3.1) Measurement of Aberrant mtDNA

According to the methods described herein, measuring the level of one or more aberrant mtDNA marker of the present invention in a biological sample can determine the presence or stage or progression of endometriosis in a subject. The present description, therefore, provides methods for detecting, diagnosing and/or monitoring endometriosis in a subject, comprising assaying a biological sample from the subject for one or more aberrant mtDNA biomarkers (or “markers”) described herein by measuring and/or quantifying the amount of the one or more aberrant mtDNA markers in the sample. Once quantified, the amount of the marker may be compared with a reference value (i.e., a control). The reference value may be based on whether the method seeks to detect, diagnose or monitor endometriosis. For example, in the case of detecting or diagnosing endometriosis, the reference value may comprise the amount of the aberrant mtDNA in a sample from a healthy subject, i.e. a subject not suffering from endometriosis. Such sample may be described herein as a “known non-endometriotic” (or “non-involved”) biological sample. Alternatively, the reference value may comprise the amount of aberrant mtDNA in a sample from a subject known to be suffering from endometriosis. Such sample may be described herein as a “known endometriotic” (or “involved”) biological sample. Where the control comprises a value, or amount, from a non-endometriotic source, it may be referred to herein as a “non-endometriotic amount”. In other aspects described herein, the control may comprise a reference value of another analyte from the same biological sample. In some cases, and as described further herein, the amount of the aberrant mtDNA may be first normalized against an amount of nuclear DNA, taken from the same subject, such as one that codes for one or more housekeeping genes, such as those coding for rRNA. In one aspect the nuclear DNA sequence used may code for the 18S rRNA. The normalized value of mtDNA could then be compared to a threshold value. In the case of detecting or diagnosing endometriosis, an increase in the amount of the subject aberrant mtDNA is indicative of endometriosis. In the case of monitoring endometriosis, biological samples may be taken over time from the subject and compared over a given time period. An increase in the amount of one or more of the herein described aberrant mtDNA over time indicates the development, recurrence, or advancement of endometriosis in the subject.

The presently described methods also encompass assaying a biological sample for a panel of aberrant mtDNA markers described herein, wherein such panel comprises two or more of the subject mtDNA markers. For example, such panel may comprise 2, 3, 4, 5, 6, 7, 8, 9, 10, or more of the presently described mtDNA markers.

In one aspect, there is provided herein a method of detecting endometriosis in a mammal, the method comprising assaying a biological sample (such as blood, menstrual fluid, a tissue sample etc.) from the mammalian subject for the presence of aberrant mtDNA by hybridizing the sample with at least one hybridization probe that is capable of recognizing, or hybridizing to, a mutant mtDNA sequence as described herein. In particular, and as described herein, such probe is provided with a nucleotide sequence that is adapted to hybridize with a portion of a mtDNA molecule of the sample, wherein such portion includes a junction point described herein.

In some aspects, the present methods comprise assaying a biological sample from a mammal by hybridizing the sample with at least two primers adapted to hybridize to an aberrant mtDNA molecule as described herein. In one aspect, one of the primers may be designed with a nucleotide sequence that is complementary to a portion of mtDNA having a junction point as described herein. In another aspect, the primers may be provided with nucleotide sequences that hybridize to regions adjacent the mtDNA junction point and adapted to overlap the junction point.

In another aspect, the present description provides a method for detecting endometriosis, wherein the assay comprises:
a) conducting a hybridization reaction using at least one of the probes described herein to allow the at least one probe to hybridize to an aberrant mitochondrial DNA sequence extracted from a biological sample;
b) quantifying the amount of the at least one aberrant mitochondrial DNA sequence in the sample by quantifying the amount of the mitochondrial DNA hybridized to the at least one probe; and,
c) comparing the amount of the mitochondrial DNA in the sample to at least one known reference value, wherein:
   if the reference value comprises an amount of mtDNA not associated with endometriosis, a higher amount of aberrant mtDNA in the sample indicates the presence of endometriosis; or,
   if the reference value comprises an amount of mtDNA associated with endometriosis, a lower amount of aberrant mtDNA in the sample indicates the absence of endometriosis.

Methods and screening tools for diagnosing endometriosis by identifying specific mitochondrial mutations are also herein contemplated. Any known method of hybridization may be used to carry out such methods including, without limitation, probe- and/or primer-based technologies including branched DNA and qPCR, both single-plex and multi-plex. Array technology, which has oligonucleotide probes matching the wild type or mutated region, and a control probe, may also be used. Commercially available arrays such as microarrays or gene chips are suitable for use with the presently described methods.

Thus, by detecting the aberrant mtDNA molecules described herein in a biological sample, it is possible to detect or diagnose endometriosis in a subject. Further, by measuring and comparing, either qualitatively or quantitatively, the amount of aberrant mtDNA in successive samples from a subject over time, the progression of endometriosis in such subject can be monitored.

### 3.2) Measurement of Fusion Transcripts

Measuring the level of the herein described mitochondrial fusion transcripts in a biological sample can also determine the presence or stage or progression of endometriosis in a subject. Thus, there is provided methods for detecting, diagnosing, and/or monitoring endometriosis, comprising extracting mitochondrial RNA from one or more biological samples obtained from a subject, and assaying the samples for fusion transcripts corresponding to the aberrant mtDNA described herein. Such assaying may comprise quantifying the amount of one or more fusion transcripts in the sample and comparing the amount detected with a reference value. The reference value is based on whether the method seeks to diagnose or monitor endometriosis. Accordingly, the reference value may relate to transcript data collected from one or more known non-endometriotic biological samples, from one or more known endometriotic biological samples, a population of known non-endometriotic or known endometriotic samples, and/or from one or more biological samples taken from the subject over time.

In one aspect, the methods described herein encompass assaying one or more biological samples from a subject for a panel of fusion transcript markers indicative of endometriosis, wherein the panel comprises 2, 3, 4, 5, 6, 7, 8, 9 or 10 RNA markers described herein.

Thus, in one aspect, there is provided a method of detecting endometriosis in a mammal, the method comprising assaying a biological sample (such as blood, menstrual fluid, or tissue) from said mammal for the presence of at least one fusion transcript described herein by hybridizing said sample with at least one hybridization probe having a nucleic acid sequence complementary to at least a portion of the mitochondrial fusion transcript, wherein the portion includes a fusion junction in the mitochondrial fusion transcript.

In another aspect, there is provided methods comprising assaying a biological sample from the mammal by hybridizing the sample with at least two primers. As discussed above, at least one of the primers may have a sequence that allows hybridization to a portion of the fusion transcript including the fusion junction. In other aspects, the primers may have sequences that allow hybridization to flanking regions of the fusion junction.

In another aspect, the invention provides a method as above, wherein the assay comprises:

a) conducting a hybridization reaction using at least one of the above-noted probes to allow the at least one probe to hybridize to a complementary mitochondrial fusion transcript;
b) quantifying the amount of the at least one mitochondrial fusion transcript in the sample by quantifying the amount of the transcript hybridized to the at least one probe; and,
c) comparing the amount of the mitochondrial fusion transcript in the sample to at least one known reference value, wherein:
   if the reference value comprises an amount of mitochondrial fusion transcript not associated with endometriosis, a higher amount of it in the sample indicates the presence of endometriosis; and
   if the reference value comprises an amount of mtDNA associated with endometriosis, a lower amount of aberrant mtDNA in the sample indicates the absence of endometriosis.

### 3.3) Detection of Translated Proteins

Translation products, proteins, of the fusion transcripts described herein may be detected using commonly known methods, such as immunological assays utilizing antibodies or other such specific binding components. In particular, such components specifically bind to the translated fusion site or junction of mtDNA

### 4.0) Kit

The present description encompasses diagnostic or screening kits for the in vitro detection, diagnosis, and/or monitoring of endometriosis of a subject. Such kits preferably include one or more probes or primers as described herein, optionally in combination with reagents, instructions, tools, and/or containers etc., as may be needed for conducting an assay.

The kits can include reagents required to conduct a diagnostic assay, such as buffers, salts, detection reagents, anticoagulating agents, and the like. Other components, such as buffers and solutions for the isolation and/or treatment of a biological sample, may also be included in the kit. One or more of the components of the kit may be lyophilised and the kit may further comprise reagents suitable for the reconstitution of the lyophilised components.

Where appropriate, the kits described herein may also contain sampling means, reaction vessels, mixing vessels, and/or other components to facilitate the collection and/or preparation of the test sample. The kit may also optionally include instructions for use, which may be provided in paper form or in computer-readable form, such as a disc, CD, DVD or the like.

In one aspect, the description provides a kit for conducting an in vitro assay for detecting and/or diagnosing endometriosis comprising a hybridization probe described herein and at least one reagent for conducting the assay.

In one aspect, a kit described herein comprises at least one hybridization probe complementary to at least a portion of an aberrant mtDNA described herein or at least a portion of a mitochondrial RNA fusion transcript described herein. As discussed above, in one aspect, the portion of the sequence to which the probe hybridizes comprises a junction point, or fusion junction, in the mtDNA or the fusion transcript. In one aspect, the kit may comprise one or more probes that are adapted to hybridize to one or more control sequences.

In another aspect, a kit described herein comprises a pair of primers, such as forward and reverse primers, for amplifying at least a portion of an aberrant mtDNA described herein or a least a portion of a mitochondrial RNA fusion transcript described herein. In one aspect, at least one of the primers has a nucleotide sequence that is adapted to hybridize to a junction point, or fusion junction, in the mtDNA or the fusion transcript. In another aspect, at least one of the primers has a nucleotide sequence that is adapted to hybridize to a sequence of the mtDNA or the fusion transcript that is adjacent to the junction point, or fusion junction, in the mtDNA or the fusion transcript. In one aspect, the kit may comprise one or more primers or primer pairs that are adapted to hybridize to one or more control sequences.

5.0) Exemplary mtDNA Mutations Fusion Transcripts, Translation Products Probes, and Primers

Described below are mtDNA mutations (or aberrant mtDNA) and fusion transcripts that have been found to be useful for the presently claimed methods. Putative translation products are also provided and are believed to also be useful for the same reason. Also provided below are probe and primer sequences that are useful for detecting the subject mtDNA and fusion transcripts.

5.1) Exemplary mtDNA Mutations

Table 1 lists the aberrant mtDNA molecules (i.e. mtDNA molecules having a deletion) that were studied. The listed sequences are based on modifications of the wild type mitochondrial genome (SEQ ID NO: 1) and have been assigned a fusion or “FUS” designation. Where provided, “AltMet” refers to alternate translation start site. The sequences listed in Table 1 are sections of the mtDNA genome that are rejoined, or re-circularized after removal oft subject deletion.

TABLE 1
mtDNA aberrations

| | | | | | | Location of |
|---|---|---|---|---|---|---|
| | | Deletion Name | | | | flanking |
| | | (Location of | | | | repeats |
| | SEQ | Deletion, with | | | Junction site | (with reference |
| Deletion | ID | reference to SEQ | Spliced | Location on | (splice location on | to SEQ ID NO: |
| ID | NO: | ID NO: 1) | Genes | mtDNA | SEQ ID) | 1) |
| 1 | 2 | FUS 8469:13447 | (ATP8) to | 8366-14148 | 8366-8469/13447- | [8470-8482] |
| | | (AltMet) | (ND5) | | 14148 | and |
| | | (“5.0 kb Deletion” | | | (nucleotides 81-82 of | 13447-13459 |
| | | or | | | SEQ ID NO: 2) | |
| | | 4977 kb “common | | | | |
| | | deletion”) | | | | |
| | | (8483-13446) | | | | |
| 4 | 3 | FUS 7992:15730 | (CO2) to | 7586-15887 | 7586-7992/15730- | 7986-7992 |
| | | (“7.7 kb Deletion”) | (Cytb) | | 15887 | and |
| | | (7993-15722) | | | (nucleotides 407-408 | [15723-15729] |
| | | | | | of SEQ ID NO: 3) | |
| 14 | 4 | FUS 9191:12909 | (ATP6) to | 8527-14148 | 8527-9191/12909- | [9192-9195] |
| | | (9196-12908) | (ND5) | | 14148 | and |
| | | | | | (nucleotides 665-666 | 12909-12911 |
| | | | | | of SEQ ID NO: 4) | |
| 14a | 5 | FUS 9188:12906 | (ATP6) to | 8527-14148 | 8527-9188/12906- | [9189-9195] |
| | | (“3.7 kb Deletion”) | (ND5) | | 14148 | and |
| | | (9196-12905) | | | (nucleotides 662-663 | 12906-12912 |
| | | | | | of SEQ ID NO: 5) | |
| 16 | 6 | FUS 10367:12829 | (ND3) to | 10059-14148 | 10059-10367/12829- | 10365-10367 |
| | | (10368-12825) | (ND5) | | 14148 | and |
| | | | | | (nucleotides 309-310 | [12826-12828] |
| | | | | | of SEQ ID NO: 6) | |
| 120 | 7 | FUS 6260:12814 | (CO1) to | 5904-14148 | 5904-6260/12814- | [6261-6270] |
| | | (“6.5 kb Deletion”) | (ND5) | | 14148 | and |
| | | (6271-12813) | | | (nucleotides 357-358 | 12814-12823 |
| | | | | | of SEQ ID NO: 7) | |
| 122 | 8 | FUS 7973:9023 | (CO2) to | 7586-9207 | 7586-7973/9023- | [7974-7983] |
| | | (“1.0 kb Deletion”) | (ATP6) | | 9207 | and |
| | | (7984-9022) | | | (nucleotides 387-388 | 9023-9032 |
| | | | | | of SEQ ID NO: 8) | |
| 193 | 9 | FUS 9086:10313 | (ATP6) to | 8527-10404 | 8527-9086/10313- | 9078-9086 |
| | | (“1.2 kb Deletion”) | (ND3) | | 10404 | and |
| | | (9087-10303) | | | (nucleotides 560-561 | [10304-10312] |
| | | | | | of SEQ ID NO: 9) | |
| 400 | 10 | FUS 9079:14988 | (ATP6) to | 8527-15887 | 8527-9079/14988- | [9080-9085] |
| | | (9086-14987) | (CYTB) | | 15887 | and |
| | | | | | (nucleotides 553-554 | 14988-14903 |
| | | | | | of SEQ ID NO: 10) | |
| 516 | 11 | FUS 7260:15540 | (CO1) to | 5904-15887 | 5904-7260/15540- | 7253-7260 |
| | | (7261-15531) | (CYTB) | | 15887 | and |
| | | | | | (nucleotides 1357- | [15532-15539] |
| | | | | | 1358 of SEQ ID NO: | |
| | | | | | 11) | |
| 586 | 12 | FUS 8431:10841 | (ATP8) to | 8366-12137 | 8366-8431/10841- | [8432-8439] |
| | | (“2.4 kb Deletion”) | (ND4) | | 12137 | and |
| | | (8440-10840) | | | (nucleotides 66-67 of | 10841-10848 |
| | | | | | SEQ ID NO: 12) | |
| 8590 | 74 | FUS 8984:13833 | (ATPase6) | 8527-14101 | 8527-8984/13833- | [8985-8993] |
| | | (“4.8 kb Deletion”) | to (ND5) | | 14101 | and |
| | | (8994-13832) | | | (nucleotides 457-458 | 13833-13841 |
| | | | | | of SEQ ID NO: 74) | |
| 2767 | 75 | FUS 5362:14049 | (ND2) to | 4470-14101 | 4470-5362/14049- | [5363-5376] |
| | | (“8.7 kb Deletion”) | (ND5) | | 14101 | and |
| | | (5377-14048) | | | (nucleotides 893-894 | 14049-14062 |
| | | | | | of SEQ ID NO: 75) | |

In Table 1, “Deletion ID” is a reference that identifies the mtDNA deletion from among those screened. “SEQ ID NO” indicates the nucleotide sequence identifier ascribed herein to the subject mtDNA deletion. “Deletion Name” identifies the “FUS” designation, wherein A:B represents the junction point between the last mitochondrial nucleotide of the first spliced gene and the first mitochondrial nucleotide of the second spliced gene. The “Location of Deletion” identifies the portion of the respective sequence that is deleted from the parent mtDNA molecule. The following column, “Spliced Genes”, identifies the spliced genes resulting from the deletion. In this regard, ATP8 represents ATPase8, ATP6 represents ATPase6, CO2 represents COII, and CO1 represents COI. The “mtDNA Location” identifies the segment of the mtDNA sequence corresponding to the wild type mtDNA genome (i.e. SEQ ID NO: 1). The “Junction Site” identifies the location of the junction point of the mutated mtDNA following removal of the deletion (based on the wild type mtDNA genome, SEQ ID NO: 1). Thus, byway of example, for Deletion ID No. 4 (SEQ ID NO: 3), having a “Junction Site” 7586-7992/15730-15887, the deleted mtDNA segment includes nucleotides 7993 to 15729. In such case, the aberrant mtDNA, once re-circularized, comprises a junction at nucleotides 7992 and 15730. The portion in brackets in this column identifies the location of the splice in the respective SEQ ID NO. The final column identifies the repeat sequences flanking the deletion. The repeats shown in square brackets are deleted along with the deletion shown in the third column.

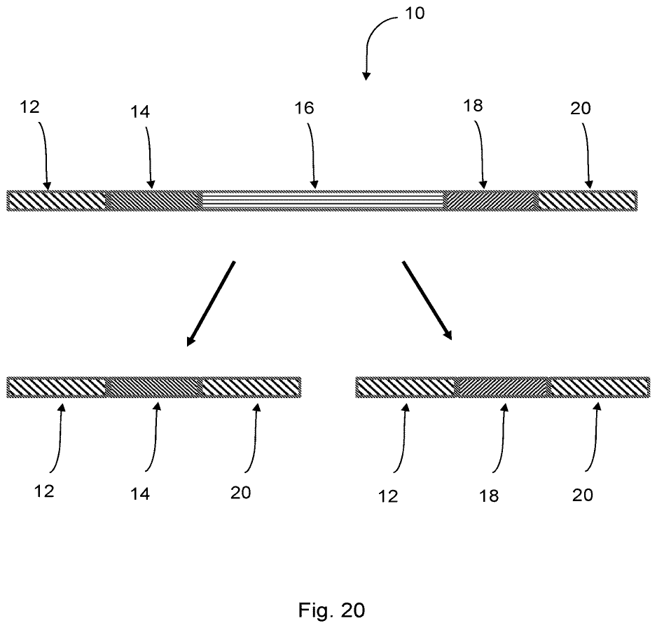

As shown in Table 1, one of the flanking repeat sequences is removed along with, and therefore forms part of, the deleted sequence. It is, however, possible that the other of the repeat sequences may be included with the deletion instead. This deletion mechanism is illustrated in FIG. 20, which shows a parent mtDNA molecule 10, wherein 12 and 20 represent the opposed ends of the mtDNA molecule and 16 represents the deletion, or deleted sequence (such as recited in the third column of Table 1). The repeat sequences are represented at 14 and 18. During the deletion event, one of the repeats 14 or 18 is deleted along with, and therefore forms part of, the deletion 16. As such, the remaining parent mtDNA, once re-circularized, will comprise segments 12-18-20 or segments 12-14-10, as illustrated in FIG. 20. Although, as noted above, one entire repeat is described as being included with the deleted sequence, there is a possibility that the only a portion of one or both of the repeats may be included with the deletion.

Mutant mtDNA sequences according to the present description may comprise any modification that results in the generation of a fusion transcript. Non-limiting examples of such modifications include insertions, translocations, deletions, duplications, recombinations, rearrangements, or combinations thereof.

The step of detecting the presently described mtDNA mutations can be selected from any technique known to those skilled in the art. For example, analyzing mtDNA can comprise selection of targets by branching DNA, sequencing the mtDNA, amplifying mtDNA by PCR, Southern, Northern, Western, South-Western blot hybridizations, denaturing HPLC, hybridization to microarrays, biochips or gene chips, molecular marker analysis, biosensors, melting temperature profiling or a combination of any of the above.

Variants or fragments of the mtDNA sequences identified herein are also contemplated. The present description encompasses the use of variants or fragments of these sequences for diagnosing and/or monitoring endometriosis.

### 5.2) Exemplary Fusion Transcripts

Exemplary fusion transcripts for use in the methods described herein are provided in Table 2. These fusion transcripts were detected and found to be useful in detecting, diagnosing and/or monitoring endometriosis as indicated in the Examples.

TABLE 2
Fusion transcripts of the present invention.

| | mtDNA | | | | |
|---|---|---|---|---|---|
| Deletion | Deletion | Transcript | Fusion Transcript | | Deletion |
| ID | SEQ ID NO | SEQ ID NO | Name | Flanking Genes | Junction |
| 1 | 2 | 13 | FUS 8469:13447 | (ATP8) to (ND5) | 8469:13447 |
| | | | (AltMet) | | |
| 4 | 3 | 14 | FUS 7992:15730 | (CO2) to (Cytb) | 7992:15730 |
| 14 | 4 | 15 | FUS 9191:12909 | (ATP6) to (ND5) | 9191:12909 |
| 14a | 5 | 16 | FUS 9188:12906 | (ATP6) to (ND5) | 9188:12906 |
| 16 | 6 | 17 | FUS 10367:12829 | (ND3) to (ND5) | 10367:12829 |
| 120 | 7 | 18 | FUS 6260:12814 | (CO1) to (ND5) | 6260:12814 |
| 122 | 8 | 19 | FUS 7973:9023 | (CO2) to (ATP6) | 7973:9023 |
| 193 | 9 | 20 | FUS 9086:10313 | (ATP6) to (ND3) | 9086:10313 |
| 400 | 10 | 21 | FUS 9079:14988 | (ATP6) to (CYTB) | 9079:14988 |
| 516 | 11 | 22 | FUS 7260:15540 | (CO1) to (CYTB) | 7260:15540 |
| 586 | 12 | 23 | FUS 8431:10841 | (ATP8) to (ND4) | 8431:10841 |
| 8590 | 74 | 76 | FUS 8984:13833 | (ATPase6) to (ND5) | 8984:13833 |
| 2767 | 75 | 77 | FUS 5362:14049 | (ND2) to (ND5) | 5362:14049 |

In Table 2: “Transcript Number” is identification number assigned to the fusion transcript and also corresponds to the mtDNA deletion ID number of Table 1. “mtDNA Deletion SEQ ID NO is the mtDNA deletion sequence identifier from Table 1. “Transcript SEQ ID NO” is the sequence identifier of the subject fusion transcript. “Fusion Transcript Name” identifies the “FUS” designation, wherein A:B represents the junction point between the last mitochondrial nucleotide of the first spliced gene and the first mitochondrial nucleotide of the second spliced gene. “Flanking Genes” identifies the spliced genes resulting from the deletion. “Deletion Junction” identifies the location of the junction point of the mtDNA molecule after removal of the deletion.

Naturally occurring fusion transcripts can be extracted from a biological sample and identified according to any suitable method known in the art, such as those methods described in the examples of the present description.

Fusion transcripts can also be produced by recombinant techniques known in the art. Typically, this involves transformation (including transfection, transduction, or infection) of a suitable host cell with an expression vector comprising an mtDNA sequence of interest.

Variants or fragments of the fusion transcripts identified herein are also contemplated.

### 5.3) Exemplary Translation Products of Fusion Transcripts

Putative amino acid sequences corresponding to transcripts of the mtDNA deletions 1, 4, 14, 16, 120, 122, 193, 400, 516, 586, 8590, and 2767 are provided in Table 3.

TABLE 3
Putative amino acid sequences corresponding to the fusion
transcripts of the present description

| Deletion | | mtDNA Deletion | Transcript | Amino Acid |
|---|---|---|---|---|
| ID | Deletion Name | SEQ ID NO | SEQ ID NO | SEQ ID NO |
| 1 | FUS 8469:13447 | 2 | 13 | 24 |
| | (AltMet) | | | |
| 4 | FUS 7992:15730 | 3 | 14 | 25 |
| 14 | FUS 9191:12909 | 4 | 15 | 26 |
| 14a | FUS 9188:12906 | 5 | 16 | 27 |
| 16 | FUS 10367:12829 | 6 | 17 | 28 |
| 120 | FUS 6260:12814 | 7 | 18 | 29 |
| 122 | FUS 7973:9023 | 8 | 19 | 30 |
| 193 | FUS 9086:10313 | 9 | 20 | 31 |
| 400 | FUS 9079:14988 | 10 | 21 | 32 |
| 516 | FUS 7260:15540 | 11 | 22 | 33 |
| 586 | FUS 8431:10841 | 12 | 23 | 34 and/or 84 |
| 8590 | FUS 8984:13833 | 74 | 76 | 78 |
| 2767 | FUS 5362:14049 | 75 | 77 | 79 |

### EXAMPLES

The following examples are provided to further illustrates aspects of the present description. The examples are not intended to limit the scope of the description in any way.

### Example 1: Large Scale Fusion Transcript Screening in Endometrial Tissue

268 probes corresponding to fusion transcripts were screened on endometrial tissue samples for evidence of differential expression in samples obtained from patients with endometriosis relative to control samples. Screening methods and results are described herein below.

### Generation of Probe Library

The 268 probes were identified using a proprietary nucleotide base pair repeat finding program. The program identified over 16000 potential deletions based on direct and indirect repetitive elements which flank the sequence to be deleted at the 5 and 3 end. The selection of the 268 probes was based on the criteria that a minimum of 8 base pair repeats were required; however, deletions with fewer than 8 base pair repeats are also possible. By way of example, the repeat for deletion 16 is 3 bp.

### Tissue Samples

Large endometrium samples (>0.49 g) were obtained. The “state” or diagnosis of the tissue as determined by the physician upon surgery as well as the reason(s) for surgery are indicated in Table 4.

TABLE 4
Tissue samples.

| Sample ID | State/Reason for surgery |
|---|---|
| 4360 | Endometriosis (ovarian cyst) |
| 3461 | Endometriosis (ovarian endometrioma) |
| 3462 | Endometriosis (ovarian cyst, pain) |
| 3463 | Control (had endometriosis 7-8 yrs prior to surgery; |
| | not present at time of surgery for possible tubal ligation) |
| 3464 | Control (uterine fibroid) |
| 3465 | Control (possible tubal reversal) |
| 3466 | Control (ablation of suspected endometriosis lesions) |

Using the samples listed in Table 4, tissue homogenates were prepared using the QuantiGene™ Sample Processing Kit for “Fresh or Frozen Animal Tissues”. For each sample, 4 portions of frozen endometrial tissue were cut and weighed (approximately 100 mg each) before being added to 6 mL of homogenizing solution containing 60 μL proteinase K. Samples were homogenized using Qiagen's Tissue Rupture probe then incubated at 65 °C overnight. Homogenates were then clarified by centrifuging twice at 16000×g for 15 minutes. The supernatant was conserved and utilized as the template for the subsequent branched DNA assay. Alternatively, DNA was extracted from the tissue homogenates or directly from fresh frozen tissue according to the protocol for tissue using Qiagen's QiaAmp™ DNA Mini Kit. DNA was then quantified on the Nanodrop™ spectrophotometer and normalized for subsequent use in a qPCR reaction.

Mitochondrial DNA deletions and resulting fusion transcripts can be detected using one of many molecular techniques. Herein, branched-DNA and quantitative PCR technologies were employed for the detection of fusion transcripts and the parent aberrant mtDNA molecules, respectively.

### Branched DNA Platform

Panomics' Quantigene™ 2.0 protocol for “Capturing Target RNA from Fresh, Frozen, or FFPE Tissue Homogenates” was followed for tissue samples. A working probe set comprised of water, lysis solution, blocking reagent and probe was first added to the capture plate. The probes (or “capture probes”) used in the present example comprise oligonucleotides that were designed (with complementary nucleotide sequences) to bind to the junction points of the mtDNA encoding the respective fusion transcripts listed in Table 2 above. In particular, the probes used in the branched DNA (bDNA) analysis were those listed in Table 5 below.

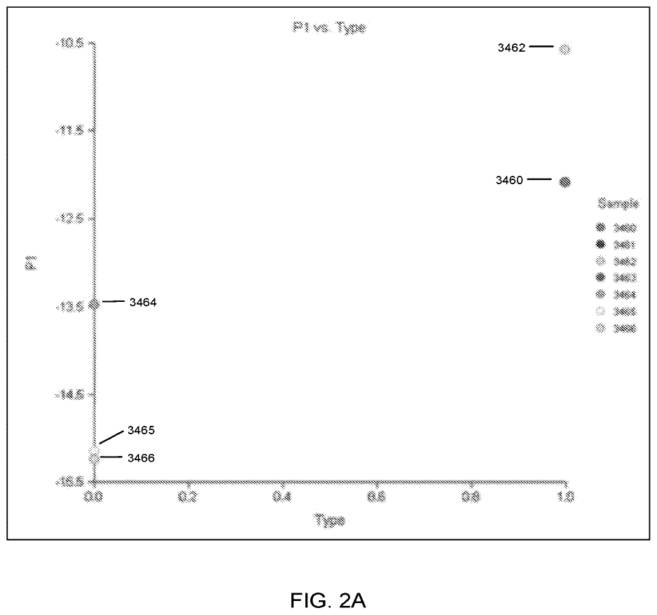

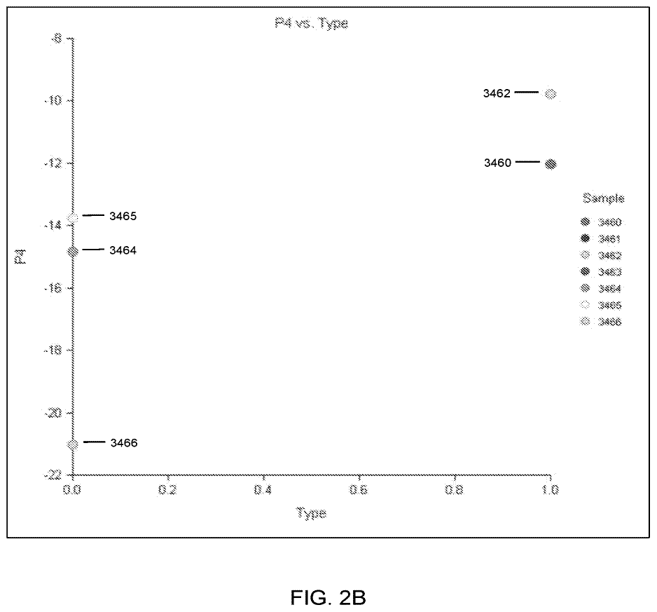

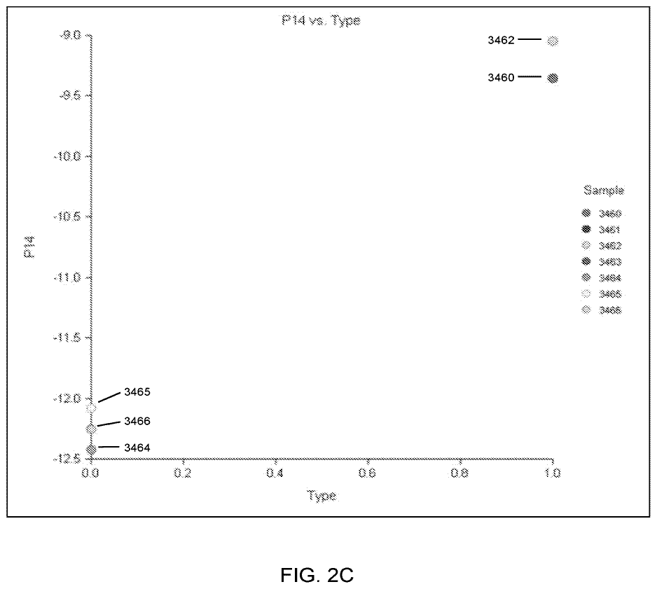

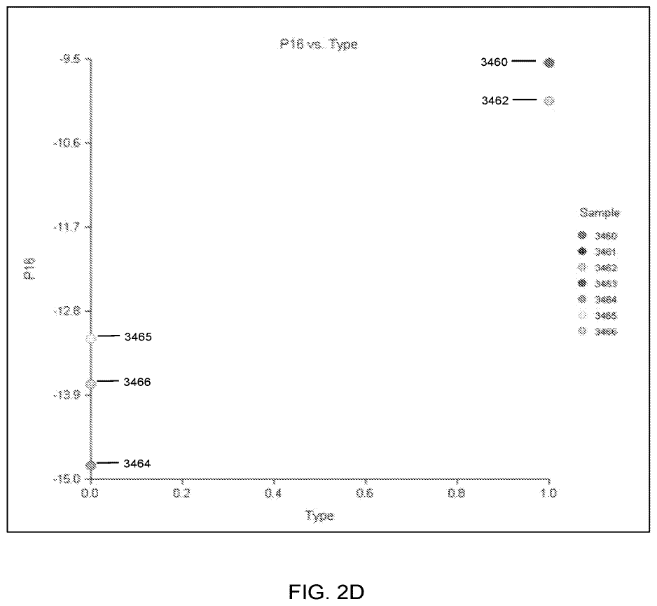

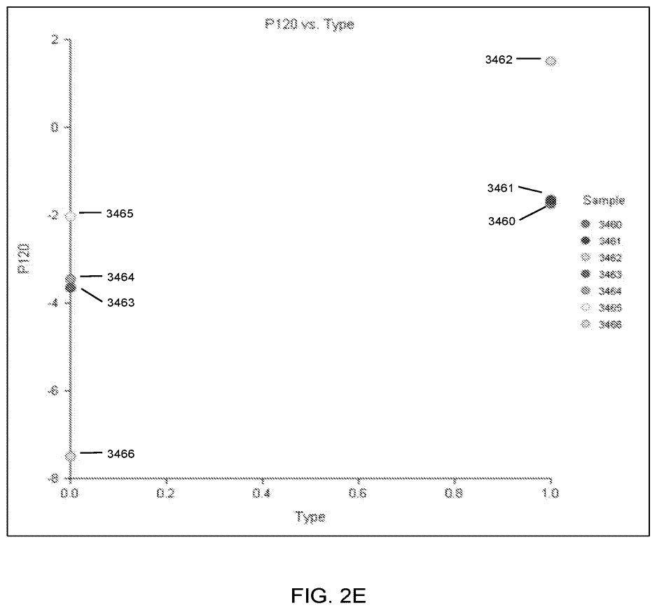

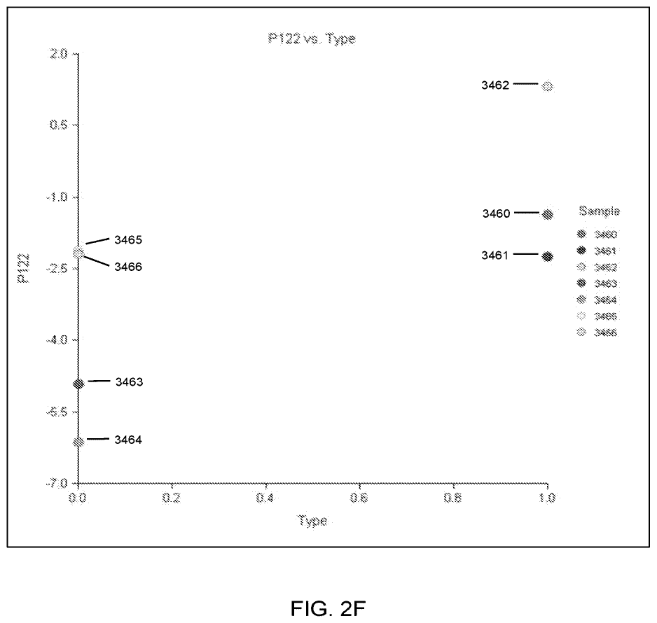

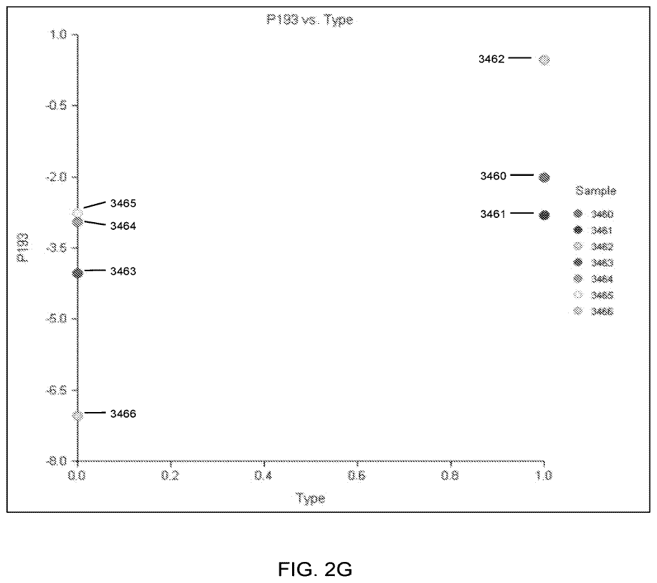

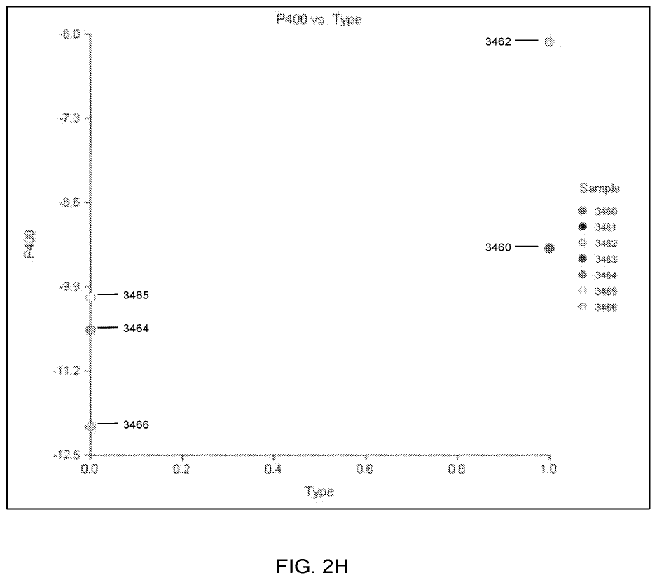

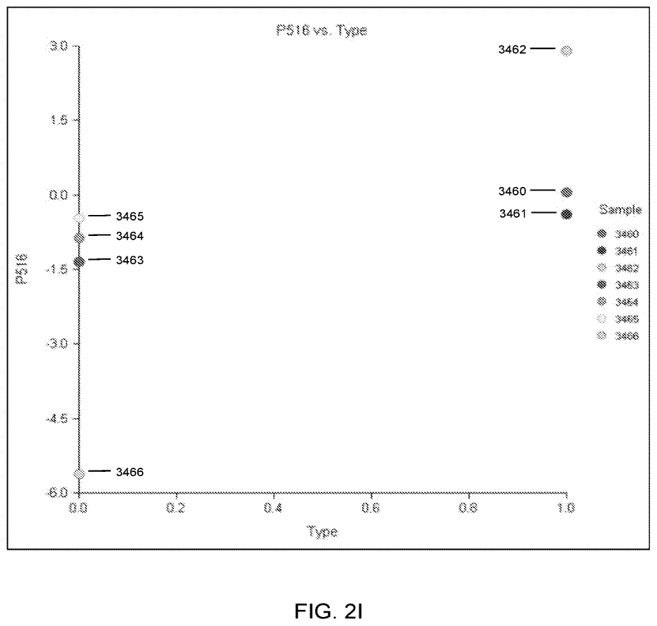

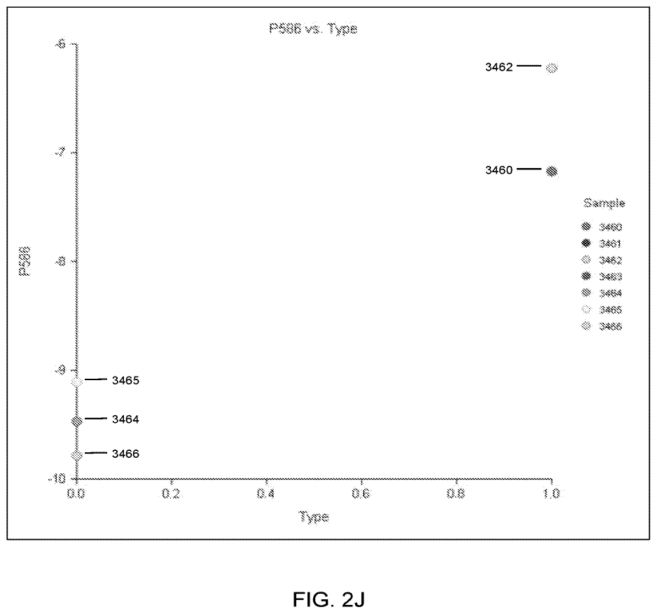

Homogenate was then added to the capture plate and incubated overnight at 55 °C to enable probe-template hybridization. Following a series of wash and hybridization steps, the chemiluminescent substrate was added. Degradation of alkaline phosphatase conjugated to the probe-template hybrid results in a luminescent signal which is reported as relative luminescence units (RLUs). Each capture plate was read in duplicate and the RLUs were measured on the Promega Glomax™ luminometer. RLU values were analyzed bioinformatically. Duplicate plate reads as well as triplicate values were each averaged, providing they had a CV (coefficient of variation; i.e. the ratio of the standard deviation to the mean) of 15%. Also, it was determined whether or not the RLU values were above background for the given probe. Specifically, the Lower Limit of Quantitation, LOQ (LOQ=the RLU average of the probes' background plus 10 standard deviations of the background average), was calculated and subtracted from the sample RLU. The sample RLU values were then transformed to log 2 or log 10 values for ease of analysis. Finally, for any given probe, the sample RLU was normalized against the housekeeper (HK) RLU by subtracting or dividing it from the sample RLU. Normalized results testing probes 1, 4, 14, 16, 120, 122, 193, 400, 516 and 586 (wherein the probe numbers correspond with the fusion transcript numbers provided above) on 3 endometriosis-positive and 4 control endometrial samples are shown in FIGS. 2A to 2J and summarized below in Table 5.

TABLE 5
Performance of fusion transcripts

| | | | Mean | | |
|---|---|---|---|---|---|
| | Mean | Mean | Difference | | |
| | Log2LOQProbe − | Log2LOQProbe − | between | | |
| Transcript/ | Log2LOQHK23 | Log2LOQHK23 | Control | Significance, | Mean Copy |
| Probe | (Control) | (Endo. Pos.) | and Endo. Pos. | P2 | # |
| 1 | −14.61 | −11.32 | −3.29 | 0.007 | 46.16 |
| 4 | −16.54 | −10.9 | −5.64 | 0.12 | 89.99 |
| 14 | −12.25 | −9.2 | −3.05 | 0.01 | 52.63 |
| 16 | −13.91 | −9.8 | −4.12 | 0.01 | 64.9 |
| 120 | −4.15 | −0.62 | −3.53 | 0.08 | 159082.93 |
| 122 | −3.84 | −0.76 | −3.08 | 0.09 | 159914.28 |
| 193 | −4.2 | −1.45 | −2.75 | 0.11 | 117468.11 |
| 400 | −10.9 | −7.71 | −3.19 | 0.27 | 1074.81 |
| 586 | −9.45 | −6.7 | −2.76 | 0.07 | 2489.43 |
| 516 | −2.07 | 0.86 | −2.93 | 0.12 | 550075.23 |

Table 5 indicates the average normalized RLU values (Log2LOQ Probe-Log2LOQHK23) for control and endometriosis positive (“Endo. Pos.”) tissue samples corresponding to scatter plots shown in FIGS. 2A to 2J. The mean difference between the two tissue groups as well as the significance of the difference is provided. The average number of copies of the given fusion transcript (i.e. probes 1, 4, 14, 16, 400, 586, 120, 122, 193 and 516) is also shown in Table 5.

qPCR Reactions

qPCR analysis was performed for transcript numbers 1, 4, 14, 16, 120, 122, 193, 586, 8590, and 2767 (see Table 6 below). Purified DNA extracts were normalized to a concentration of 0.25 ng/μL using nuclease-free ultrapure water. qPCR reactions were set-up at room temperature under dim light using Qiagen's Quantitect™ Sybr Green® PCR kit. 10 μL of template was added to 12.5 μL of the 2× master mix along with 0.025-0.0625 μL of each 100 μM forward and reverse primers, depending on the target. Primer sequences were designed for the specific DNA targets and these sequences are shown in Tables 6 (junction primers) and 7 (flanking primers). As used herein, the term “junction primer” will be understood to mean a primer that hybridizes to a region of the target DNA molecule having at least one of the pair of nucleotides forming the junction point after removal of the deletion. Thus, in one aspect, the junction primer may overlap both nucleotides forming the junction point or only one of such nucleotides. As shown in the tables below, more than one set of primers was used in some cases. The reaction was made up to a final volume of 25 μL using PCR-grade H₂O. Reaction mixtures were cycled on either the Chromo 4™ (Biorad) or Opticon 2™ (MJ Research) real-time PCR cyclers.

TABLE 6
Junction primer sequences used in qPCR reactions to target large-scale mtDNA
deletions

| Deletion | | | | | |
|---|---|---|---|---|---|
| ID | Direction | Sequence (5′-3′) | Positions | Seq. Contribution | Length |
| 1 | Fwd | TCTACCCCCTCTAGAGCCC | 8277-8300 | | 24 |
| (8469: | | ACTGT | | | |
| 13447) | | (SEQ ID NO: 35) | | | |
| | Rev | CTAGGCTGCCAATGGTGA | 13741-13460 | CTAGGCTGCCAA | 25 |
| | | GGGAGGT | | | |
| | | Junction | 8482-8470 | TGGTGAGGGAGGT | |
| | | (SEQ ID NO: 36) | | | |
| 4 | Fwd | TGCGACTCCTAGCCGCAG | 7983-7992 | TGCGACTCCT | 30 |
| (set 1) | 7983F | ACCTCCTCATTC | | | |
| (7992: | | Junction | 15730-15749 | AGCCGCAGACCTCCTC | |
| 15730) | | (SEQ ID NO: 37) | | ATTC | |
| | Rev | GGTACCCAAATCTGCTTCC | 16053-16026 | | 28 |
| | 16053R | CCATGAAAG | | | |
| | | (SEQ ID NO: 38) | | | |
| 4 | Fwd | TGCGACTCCTAGCCGCAG | 7983-7992 | TGCGACTCCT | 20 |
| (set 2) | 7983F | AC | | | |
| | | Junction | 15730-15739 | AGCCGCAGAC | |
| | | (SEQ ID NO: 39) | | | |
| | Rev | (same as set 1) | | | |
| | 15935R | | | | |
| 4 | Fwd | CGCCATCATCCTAGTCCTC | 7792-7815 | | 24 |
| (set 3) | | ATCGC | | | |
| | | (SEQ ID NO: 40) | | | |
| | Rev | GAATGAGGAGGTCTGCGG | 15749-15730 | GAATGAGGAGGTCTGC | 27 |
| | | CTAGGAGTC | | GGCT | |
| | | Junction | 7992-7986 | AGGAGTC | |
| | | (SEQ ID NO: 41) | | | |
| 14 | Fwd | GTAAGCCTCTACCTACACT | 9178-9191 | GTAAGCCTCTACCT | 26 |
| (set 1) | 9178F | CCAACTC | | | |
| (9191: | | Junction | 12909-12920 | ACACTCCAACTC | |
| 12909) | | (SEQ ID NO: 42) | | | |
| | Rev | GCGGATGAGTAAGAAGATT | 13122-13100 | | 23 |
| | 13122R | CCTG | | | |
| | | (SEQ ID NO: 43) | | | |
| 14 | Fwd | (same as set 1) | | | |
| (set 2) | 9178F | | | | |
| (9191: | Rev | GGAGACCTAATTGGGCTG | 13024-13012 | | 23 |
| 12909) | 13024R | ATTTG | | | |
| | | (SEQ ID NO: 44) | | | |
| 14 | Fwd | GGCCGTACGCCTAACCGC | 8944-9011 | | 20 |
| (set 3) | | TA | | | |
| (9188: | | (SEQ ID NO: 45) | | | |
| 12906) | Rev | GTTGTGGGTCTCATGAGTT | 12934-12913 | GTTGTGGGTCTCATGA | 29 |
| | | GGAGTGTAGG | | GTTGGA | |
| | | Junction | 9195-9189 | GTGTAGG | |
| | | (SEQ ID NO: 46) | | | |
| 14a | Fwd | CCCTGGCCGTACGCCTAA | 8989-9009 | | 20 |
| (9188: | | CC | | | |
| 12906) | | (SEQ ID NO: 47) | | | |
| | Rev | ATTTGTTGTGGGTCTCATG | 12938-12913 | ATTTGTTGTGGGTCTC | 33 |
| | | AGTTGGAGTGTAGG | | ATGAGTTGGA | |
| | | Junction | 9195-9189 | GTGTAGG | |
| | | (SEQ ID NO: 48) | | | |
| 16 | Fwd | CCCTAAGTCTGGCCAACAC | 10354-10367 | CCCTAAGTCTGGCC | 25 |
| (10367: | 10354F | AGCAGC | | | |
| 12829) | | Junction | 12829-12839 | AACACAGCAGC | |
| | | (SEQ ID NO: 49) | | | |
| | Rev | GGGTGGAGACCTAATTGG | 13028-13007 | | 22 |
| | 13028R | GCTG | | | |
| | | (SEQ ID NO: 50) | | | |
| 122 | Fwd | CGTCTGAACTATCCTGCCC | 7773-7794 | | 21 |
| (7973: | | GC | | | |
| 9023) | | (SEQ ID NO: 53) | | | |
| | Rev | CAATTAGGTGCATGAGTAG | 9049-9033 | CAATTAGGTGCATGAG | 27 |
| | | GTGGCCTG | | T | |
| | | Junction | 7983-7974 | AGGTGGCCTG | |
| | | (SEQ ID NO: 54) | | | |
| 193 | Fwd | AAGGCACACCTACACCCCT | 8918-8937 | | 20 |
| (set 1) | | T | | | |
| (9086: | | (SEQ ID NO: 55) | | | |
| 10313) | Rev | GAGGGATGACATAACTATT | 10334-10312 | GAGGGATGACATAACT | 28 |
| | | AGTGGCAGG | | ATTAGT | |
| | | Junction | 9086-9081 | GGCAGG | |
| | | (SEQ ID NO: 56) | | | |
| 193 | Fwd | AACCAATAGCCCTGGCCGT | 8981-9000 | | 20 |
| (set 2) | | A | | | |
| (9086: | | (SEQ ID NO: 57) | | | |
| 10313) | Rev | GAGGGATGACATAACTATT | 10334-10312 | GAGGGATGACATAACT | 31 |
| | | AGTGGCAGGTTA | | ATTAGT | |
| | | Junction | 9086-9078 | GGCAGGTTA | |
| | | (SEQ ID NO: 58) | | | |
| 586 | Fwd | CTATAGCACCCCCTCTACC | 8264-8284 | | 21 |
| (8431: | | CC | | | |
| 10841) | | (SEQ ID NO: 59) | | | |
| | Rev | GATGCTAATAATTAGGCTG | 10867-10849 | GATGCTAATAATTAGG | 27 |
| | | TGGGTGGT | | CTG | |
| | | Junction | 8439-8432 | TGGGTGGT | |
| | | (SEQ ID NO: 60) | | | |
| 8590 | Fwd | TGCCCTAGCCCACTTCTTA | 8895-8915 | | 21 |
| (8984: | | CC | | | |
| 13833) | | (SEQ ID NO: 80) | | | |
| | Rev | TAGTTGAGGTCTAGGGCTG | 13853-13833 | TAGTTGAGGTCTAGGG | 25 |
| | | TTGGTT | | CTG | |
| | | Junction | 8984-8981 | GGTT | |
| | | (SEQ ID NO: 81) | | | |
| 2767 | Fwd | GGGCCATTATCGAAGAATT | 5260-5284 | | 25 |
| (5362: | | CACAAA | | | |
| 14049 | | (SEQ ID NO: 82) | | | |
| | Rev | GAGGTGATGATGGAGGTG | 14069-14049 | GAGGTGATGATGGAG | 24 |
| | | GAGTAG | | GTGGAG | |
| | | Junction | 5362-5360 | TAG | |
| | | (SEQ ID NO: 83) | | | |

TABLE 7
Flanking primer sequences used in qPCR reactions to target large-scale
mtDNA deletions

| Deletion | | | | |
|---|---|---|---|---|
| ID | Direction | Sequence (5′-3′) | Positions | Length |
| 1 | Fwd | ACAGTGAAATGCCCCAACTA | 8358-8377 | 20 |
| (8469: | | (SEQ ID NO: 61) | | |
| 13447) | Rev | GCTCAGGCGTTTGTGTATGA | 13540-13559 | 20 |
| | | (SEQ ID NO: 62) | | |
| 4 | Fwd | CAACGATCCCTCCCTTACCA | 7855-7874 | 20 |
| (7992: | | (SEQ ID NO: 63) | | |
| 15730) | Rev | AGTACGGATGCTACTTGTCCA | 15796-15816 | 21 |
| | 16053R | (SEQ ID NO: 64) | | |
| 14 | Fwd | GAAGCGCCACCCTAGCAATA | 9050-9063 | 20 |
| (9191: | | (SEQ ID NO: 65) | | |
| 12909) | Rev | GGTGAGGCTTGGATTAGCGT | 12950-12969 | 20 |
| | | (SEQ ID NO: 66) | | |
| 16 | Fwd | AATCCACCCCTTACGAGTGC | 10156-10175 | 20 |
| (10367: | | (SEQ ID NO: 67) | | |
| 12829) | Rev | (same as 14) | | |
| 120 | Fwd | ACAACGTTATCGTCACAGCCC | 6064-6084 | 21 |
| (6260: | | (SEQ ID NO: 68) | | |
| 12814) | Rev | GTGAGGCTTGGATTAGCGTT | 12949-12968 | 20 |
| | | (SEQ ID NO: 69) | | |
| 120a | Fwd | ACAACGTTATCGTCACAGCCCATGC | 6064-6088 | 25 |
| (6260: | | (SEQ ID NO: 51) | | |
| 12814) | Rev | GATTGCTTGAATGGCTGCTGTGTTGGC | 12852-12826 | 27 |
| | | (SEQ ID NO: 52) | | |
| 122 | Fwd | CTGAACCTACGAGTACACCGA | 7900-7920 | 21 |
| (7973: | | (SEQ ID NO: 70) | | |
| 9023) | Rev | GTGTGAAAACGTAGGCTTGGA | 9152-9172 | 21 |
| | | (SEQ ID NO: 71) | | |
| 193 | Fwd | TCGAAACCATCAGCCTACTCA | 8957-8977 | 21 |
| (9086: | | (SEQ ID NO: 72) | | |
| 10313) | Rev | CCAATTCGGTTCAGTCTAATCCT | 10385-10407 | 23 |
| | | (SEQ ID NO: 73) | | |

### Results and Discussion

As noted above, approximately 268 fusion transcripts were screened in the course of this study, from which 10 endometriosis markers, as discussed herein in more detail, were selected for further study. In particular, as described herein, elevated levels of fusions transcripts associated with deletion ID nos. 1, 4, 14, 16, 120, 122, 193, 400, 516, and 586 (i.e. the transcripts of SEQ ID NOs: 13-15, and 17-23, respectively) in endometrial tissue were found to be associated with endometriosis. The presence of each transcript was determined by assaying for the respective probe having a nucleotide sequence complementary to at least a portion of the transcript having a junction point.

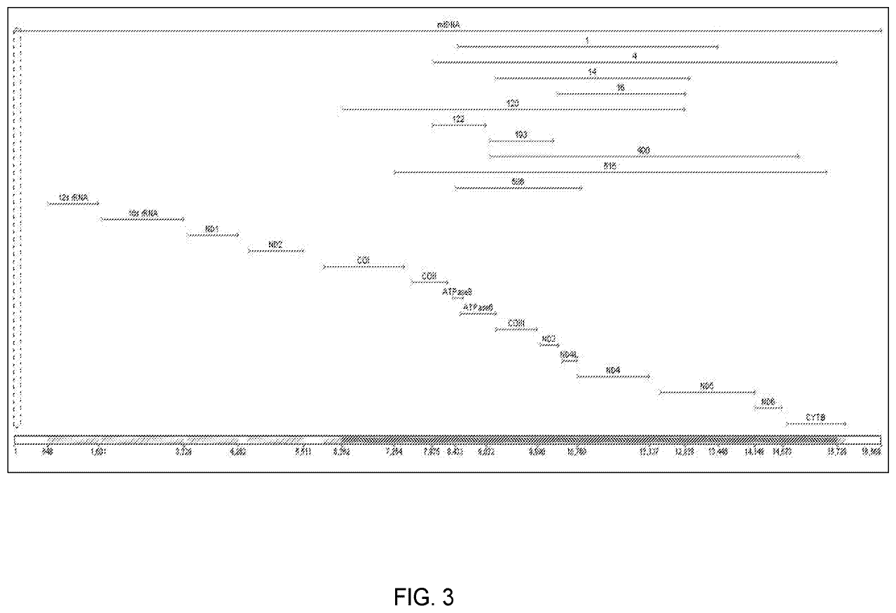

The scatterplots and performance of all fusion transcript probes are shown in FIGS. 2A to 2J and in Table 5. FIG. 3 illustrates the locations of the fusion transcripts across the mitochondrial genome, gene locations within the genome and the locations of the 10 mtDNA fusion transcripts of the present invention (i.e., “probes” or “targets”) within the genome are indicated by a line spanning the length of each deletion. Probes corresponding to the aforementioned fusion transcripts were tested against 3 endometriosis positive and 4 endometriosis negative endometrium samples (See Table 1). For each sample, the RLU values were normalized against the RLU values obtained for housekeeping gene transcripts HK23 (Human Beta-2-microglobulin), HK25 (Human GAPD) and HK18 (Peptidyl-prolyl isomerase B).

Based on the results of the present study, it is concluded that fusions transcripts 1, 4, 14, 16, 120, 122, 193, 400, 516, and 586 (i.e. the transcripts having the sequences set forth in SEQ ID NOs: 13-15, and 17-23, respectively) can be used in the detection of endometriosis, particularly by assaying endometrial tissue. In particular, in the present investigation, elevated levels of the subject transcripts in endometrial tissue samples have been found to be highly correlated with endometriosis. The detection of the subject fusion transcripts can be achieved using the probes identified above that have nucleotide sequences that are at least substantially complementary to the nucleotide sequences of at least a portion of the respective fusion transcript, wherein such portion includes a junction point, such that the probes hybridize to the respective fusion transcript.

Based on these findings, it is also concluded that elevated levels of aberrant mtDNA, having the above-identified deletions 1, 4, 14, 16, 120, 122, 193, 400, 516, and 586 (i.e. deletions having the nucleotide sequences set forth in SEQ ID NOs: 2-4, and 6-12, respectively) can be used in the detection of endometriosis. Such deletions can be identified by identifying the junction point of the parent mtDNA after re-circularization (i.e. the re-circularized large sublimon). The junction points can be identified using probes having nucleotide sequences that are at least substantially complementary to at least a portion of the mtDNA nucleotide sequences including the junction point, such that the probes hybridize to the respective mtDNA. The junction points can also be identified using primers wherein at least one of the primers has a nucleotide sequence that is substantially complementary to the mtDNA nucleotide sequence having the junction point. Alternatively, the primers may comprise pairs having nucleotide sequences that are at least substantially complementary to mtDNA sequences adjacent to the junction point.

Similarly, it can be concluded that the deletions can also be identified by identifying the junction point of the deleted sequence after re-circularization (i.e. the re-circularized small sublimon).

Translation products from the fusion transcripts (i.e. the fusion proteins having amino acid sequences set forth in SEQ ID NOs: 24-26, 28-34, and 84, respectively) may also be usable for such detection method.

Thus, as described herein a method for the detection of endometriosis is provided, wherein the method comprises the use of probes and primers for the identification of the aforementioned fusion transcripts or aberrant mtDNA. These probes and primers have nucleic acid sequences that are complementary to such mitochondrial fusion transcripts and their parent aberrant mtDNA molecules, respectively. In particular, the probes described herein are designed to be at least substantially complementary to fusion transcripts encoding a transcribed junction point corresponding to the re-joined (or re-circularized) mtDNA. The primers described herein are preferably designed so that one of the primer pairs has a nucleotide sequence that is complementary to a junction point of aberrant re-circularized mtDNA following removal of the deletions described herein. It would also be understood that other primer pairs may be designed wherein one of the primer pairs is at least substantially complementary to the junction point of the re-circularized deletion sequence or where the primer pairs are at least substantially complementary to mtDNA nucleotide sequences adjacent to the junction point.

### Example 2: Detection of mtDNA Deletions in Circulatory Blood Samples

In this example, mitochondrial DNA, mtDNA, deletions were investigated as potential biomarkers for endometriosis. the study focused primarily on mtDNA deletions obtained from circulatory blood samples. Seven deletions were investigated. Two of these deletions, the “1.2 kb Deletion” and the “3.7 kb Deletion”, discussed further below, were determined to have a high diagnostic accuracy as biomarkers using minimally-invasive blood specimens collected from women of child-bearing potential with symptoms of endometriosis. The 1.2 kb and 3.7 kb deletions were discussed above, wherein the 1.2 kb deletion was identified as deletion “193” and the 3.7 kb deletion was identified as deletion “14” or “14a”. These characteristics of these deletions were summarized earlier in Table 1 but are again provided in Table 8 for convenience. It will be understood that references herein to the “3.7 kb deletion” will be understood as references to deletion 14 or deletion 14a.

TABLE 8
mtDNA aberrations studied in Example 2

| | | | | | Junction site |
|---|---|---|---|---|---|
| Deletion | | | | mtDNA | (splice location on |
| ID | SEQ ID NO. | Deletion Name | Spliced Genes | Location | SEQ ID) |
| 14 | 4 | FUS 9191:12909 | (ATP6) to (ND5) | 8527-14148 | 8527-9191/12909- |
| | | (“3.7 kb | | | 14148 |
| | | Deletion”) | | | (nucleotides 665-666 |
| | | | | | of SEQ ID NO: 4) |
| 14a | 5 | FUS 9188:12906 | (ATP6) to (ND5) | 8527-14148 | 8527-9188/12906-14148 |
| | | (“3.7 kb | | | (nucleotides 662-663 |
| | | Deletion”) | | | of SEQ ID NO: 4) |
| 193 | 8 | FUS 9086:10313 | (ATP6) to (ND3) | 8527-10404 | 8527-9086/10313-10404 |
| | | (“1.2 kb | | | (nucleotides 560-561 |
| | | Deletion”) | | | of SEQ ID NO: 8) |

As discussed previously, the 1.2 kb deletion refers to a deletion of nucleotides 9087-10312 from the wild-type mtDNA genome (SEQ ID NO: 1). Such deletion therefore results in a large sublimon having bases 0-9086 and 10313-16568, which, when re-circularized, has a junction between nucleotides 9086 and 10313. Similarly, the 3.7 kb deletion refers to a deletion of nucleotides 9189-12905, resulting in a large sublimon having bases 0-9188 and 12906-16568, which, when re-circularized, has a junction between nucleotides 9188 and 12906.

As discussed above, although re-circularization of the large sublimon has been discussed, such re-circularization of the small sublimon is also possible, with the re-circularized small sublimon having a unique junction point as shown. In the present study, the small sublimons corresponding to the 1.2 kb and 3.7 kb deletions were identified in the course of sequencing samples. Thus, the findings in the present example can be extended to the detection of small sublimons resulting from the deletions described herein.

### Methods

### Participants and Sample Collection

This study utilized residual de-identified clinical specimens collected from prospectively enrolled patients as part of the EndOx study at Oxford Endometriosis CaRe Centre, John Radcliffe Hospital, University of Oxford. Briefly, specimens were collected from women scheduled to undergo laparoscopy for suspected endometriosis because of pelvic pain (symptomatic) or tubal ligation (asymptomatic). Study participants were female, aged 18 years or older (until menopause), and were confirmed as not pregnant. All specimens were obtained under a study protocol that received appropriate Ethics Committee approval from the National Research Ethics Service (Oxfordshire REC A, 09/H0604/58). All clinical specimens were anonymized to protect the identity of the source patient. The study was designed, implemented, and reported in accordance with the International Council for Harmonisation, Harmonised Tripartite Guidelines for Good Clinical Practice, with applicable local regulations, and the ethical principles laid down in the Declaration of Helsinki. All patients gave written informed consent prior to participation.

Blood specimens and extensive clinical phenotypic data were collected prior to surgery. Study specimens were collected, transported, and stored in accordance with the standardized WERF EPHect procedures [26, 41-44].

### Patient Populations/Study Cohorts

Clinical specimens used in this study were classified as asymptomatic controls, symptomatic controls of surgically confirmed absence of endometriosis, or cases of surgically confirmed endometriosis. Asymptomatic controls were defined as specimens collected from a patient that underwent a scheduled tubal ligation without a clinical suspicion of endometriosis, and surgically confirmed absence of endometriosis. Symptomatic controls were defined as specimens collected from patients having pain or other symptoms (excluding infertility) with a clinical suspicion of endometriosis, but no endometriosis lesions visualized by laparoscopy by experienced gynecological surgeons.

Endometriosis was scored by the operating surgeon using the revised American Society of Reproductive Medicine (rASRM) classification of endometriosis [45]. Cases were grouped by disease subtype (peritoneal, ovarian, deep endometriosis) and rASRM stage, with stages I through IV representing minimal, mild, moderate, and severe disease, respectively.

Sample Handling, Processing, and mtDNA Amplification

### DNA Extraction

Total DNA was extracted from 200 μL of plasma using the QIAamp 96 QIAcubeHT™ extraction kit (Qiagen, Crawley, UK), automated on a QIAcube HT™ system (Qiagen, Crawley, UK). Extracted DNA was eluted in 200 μL of AE buffer.

mtDNA Deletion Real-Time qPCR

Amplification was performed in 20 μL reactions using a 96-well microplate (Bio-Rad, Hemel Hempstead, UK). Each well contained 5 μL of un-normalised DNA template, 1×SYBR Green® master mix and 250 nM of the respective primers. The primers used for the reactions are provided in Table 9.

TABLE 9
Primer sequences used for amplification of 1.2 kb and 3.7 kb deletions

| | Primer | | | |
|---|---|---|---|---|
| Deletion ID | Direction | Length | Sequence | SEQ ID NO. |
| 193 | Fwd | 20 | AACCAATAGCCCTGGCCGTA | 57 |
| (“1.2 kb | Rev | 31 | GAGGGATGACATAACTATTAGTGGCAGGTTA | 58 |
| Deletion”) | (junction | | | |
| | primer) | | | |
| 14a | Fwd | 20 | GGCCGTACGCCTAACCGCTA | 45 |
| (“3.7 kb | Rev | 29 | GTTGTGGGTCTCATGAGTTGGAGTGTAGG | 46 |
| Deletion”) | (junction | | | |
| | primer) | | | |

PCR and SYBR Green® I fluorescence were analyzed using a Chromo4™ Real-time PCR Detection System (Bio-Rad, Hemel Hempstead, UK). Cycling conditions for the 1.2 kb deletion were as follows: 3 minutes at 95 °C, followed by 5 cycles of 30 seconds at 95 °C, 30 seconds at 67 °C, and 30 seconds at 72 °C; for each subsequent cycle, the annealing temperature was decreased by 0.5 °C increments. Amplification conditions were: 45 cycles of 30 seconds at 95 °C, 30 seconds at 65 °C, and 30 seconds at 72 °C. All other deletions were amplified with a standard protocol of 45 cycles of 30 seconds at 95 °C, 30 seconds at 58-65 °C, and 30 seconds at 72 °C. Following amplification, melting curve analysis was performed from 70 °C to 90 °C, reading every 0.5 °C. Each plate of samples and controls was amplified in triplicate on three separate occasions.

Real-Time qPCR Normalisation with 18S rRNA

Target amplicon quantity was normalized using the 18S rRNA nuclear DNA gene. Amplification reactions were performed as 20 μL reactions in a 96-well microplate. Each well contained 5 μL of un-normalised DNA template, 1×SYBR Green® master mix, and 200 nM of each primer. Amplification and SYBR Green® I fluorescence was analyzed using a Chromo4 Real-Time PCR Detection System. Amplification conditions were: 3 minutes at 95 °C, followed by 40 cycles of 30 seconds at 95 °C, 30 seconds at 64.5 °C, and 30 seconds at 72 °C. Following amplification, a melting curve analysis was performed from 70 °C to 90 °C, reading every 0.5 °C.

### Quality Control

The quantification cycle (Cq) was calculated using the CFX manager software regression model (Bio-Rad, Hemel Hempstead, UK). The Cq of each deletion amplicon was normalised to the Cq of the multi-copy nuclear target 18s rRNA gene amplicon. All samples were amplified in triplicate on separate plates and were considered to have passed if at least two of the three replicates were within 1.5 Cq and the melting temperature (Tm) was consistent with the target amplification product when present, (deletion Tm 81 °C±2 °C, 18S rRNA Tm 82 °C±2 °C).

Two no-template control samples were processed alongside each batch of DNA extractions and verified as negative for amplification of both the deletion target and the 18S rRNA gene. Two no-template control reactions were included on each PCR plate and verified negative for amplification of both the deletion targets and the 18s rRNA gene. Deletion primer specificity was evaluated using rho 0 cellular DNA (to detect mitochondrial pseudogene amplification) as well as DNA from healthy male buccal swabs and DNA extracted from the rho 0 parental cell line (prior to depletion of mitochondria).

For the initial round of standard PCR reactions, extracted DNA from patients with confirmed endometriosis underwent whole genome amplification using the Repli-G™ mitochondrial DNA kit (Qiagen) to ensure sufficient DNA quantity during this phase.

### Rho 0 Cell Preparation

Rho 0 cells were prepared as previously described [46]. Briefly, cells from the human osteosarcoma cell line 143B (ATCC CRL-8303) were treated with ethidium bromide to deplete cytoplasmic mitochondrial DNA. Cells were grown to confluence in high glucose DMEM with pyruvate, L-glutamine, uridine (50 μg/ml) and 5% FBS.

### Statistical Analysis

No formal sample size calculation was performed; the number of clinical specimens used was deemed sufficient to satisfy the study objective.

For qPCR, targets were amplified from all specimens in triplicate and average Cq values were calculated. The normalised deletion value (ΔCq) was determined by quantifying the deletion amplicon relative to the 18S rRNA reference amplicon. Statistical analyses were performed using Graphpad Prism™ 5.0, (Graphpad software Inc., La Jolla, CA, USA) for receiver operating characteristic (ROC) curves and descriptive statistics. SPSS v17.0 (IBM Corp., Armonk, NY, USA) was used to perform correlations and significance tests. Clinical characteristics were summarized using count and percentages for categorical data, and mean, standard deviation (SD), and range for continuous variables. The means of two groups were compared using the Student's t-test and the Mann-Whitney U test for parametric and non-parametric distributions, respectively. Correlation between the two variables was assessed with the Pearson correlation coefficient (r). With respect to the presence of endometriosis, ROC curves were constructed for all but the 6.5 kb deletion. The area under the curve (AUC) of the ROC and the sensitivity and specificity at selected cut-offs (described below) were calculated with 95% confidence intervals (CIs). A p-value <0.05 was considered statistically significant for all tests.

### Results

### Patient Population and Clinical Specimens

Demographics and clinical characteristics for patients that provided the clinical specimens used to evaluate the 1.2 kb and 3.7 kb deletions are summarized in Table 10.

TABLE 10
Demographic and Clinical Characteristics

| | 1.2 kb deletion cohort | 3.7 kb deletion cohort |
|---|---|---|
| Characteristic | N (%) | N (%) |
| TOTAL | 171 | 181 |
| Patient age¹ | 34.2 (6.8) | 34.4 (6.9) |
| Hormone therapy status² | | |
| Yes | 48 (28.1) | 55 (30.4) |
| No | 116 (67.8) | 119 (65.7) |
| Undetermined | 7 (4.1) | 7 (3.9) |
| Menstrual phase³ | | |
| No menstruation | 24 (14.0) | 26 (14.4) |
| Irregular menstruation | 12 (7.0) | 15 (8.3) |
| Menstruation | 31 (18.1) | 31 (17.1) |
| Follicular phase | 44 (25.7) | 44 (24.3) |
| Luteal phase and extended | 60 (35.1) | 65 (35.9) |
| CONTROLS | 28 | 32 |
| Patient age¹ | 36.6 (6.9) | 37.2 (6.8) |
| Symptomatic | 18 (64.3) | 19 (58.4) |
| Asymptomatic | 10 (35.7) | 13 (40.6) |
| CASES | 143 | 149 |
| Patient age¹ | 33.7 (6.7) | 33.8 (6.8) |
| Endometriosis type | | |
| Peritoneal | 49 (34.3) | 52 (34.9) |
| Ovarian | 45 (31.5) | 47 (31.5) |
| Deep infiltrating | 49 (34.3) | 50 (33.6) |
| Endometriosis stage | | |
| Stage I | 63 (44.1) | 65 (43.6) |
| Stage II | 21 (14.7) | 24 (16.1) |
| Stage III | 29 (20.3) | 30 (20.1) |
| Stage IV | 28 (18.6) | 28 (18.8) |
| Unknown | 2 (1.4) | 2 (1.3) |

Abbreviations:
N = number of patients/specimens;
SD = standard deviation.
¹Mean (SD) is presented; mean and SD were calculated for patients that provided age at time of specimen collection.
²Patients' status within 3 months of specimen collection.
³Patients' menstrual status at time of specimen collection.

The clinical and demographic characteristics of patients and specimens used for the 1.2 kb and 3.7 kb evaluations were similar and differ as a result of inclusion of only those samples with paired qPCR results for 18S rRNA and each deletion that met the acceptance criteria described previously.

### 1.2 kb Deletion Cohort

One hundred seventy-one specimens were used in the 1.2 kb deletion evaluation. The mean (SD) age of patients that provided specimens was 34.2 (6.8) years. The mean ages were similar between the control and case groups with mean (SD) ages of 36.6 (6.9) and 33.7 (6.7) years, respectively and were not statistically significantly different (p=0.113). Of the 171 patient specimens used in the evaluation, 116 (67.8%) patients reported no hormone therapy within the three months prior to specimen collection, 48 (28.1%) reported having hormone therapy within three months prior to specimen collection, and 7 (4.1%) were undetermined. Menstrual cycle phase data was calculated using the last menstrual period (LMP) prior to the date of blood collection in relation to a patient's normal cycle length. Twenty-four (14.0%) patients reported no menstruation—19 of whom were on hormones, 12 (7.0%) reported irregular menstruation, 31 (18.1%) were in the menstrual phase (between 1 to 5 days from the first day of LMP), 44 (25.7%) were in the follicular phase (5 to 14 days from LMP), and 60 (35.1%) were in the luteal+extended menstrual phase (>15 days from LMP).

The control group included a total of 28 specimens; 18 (64.3%) specimens collected from symptomatic patients (presenting with symptoms consistent with endometriosis other than infertility and surgical confirmed absence for the disease) and 10 (35.7%) specimens collected from asymptomatic patients scheduled for tubal ligation. The test group included 143 specimens from patients with three disease subtypes (peritoneal, ovarian, and deep infiltrating [DI] endometriosis) that were classified into four stages (rASRM I through IV). Forty-nine (34.3%) specimen were collected from women with peritoneal, 45 (31.5%) were from women with ovarian, and 49 (34.3%) were collected from women with deep endometriosis. Sixty-three (44.1%) specimens were from patients with stage I disease, 21 (14.7%) were stage II, 29 (20.3%) were stage Ill, and 28 (18.6%) were stage IV. Two (1.4%) specimens had an unknown disease stage.

### 3.7 kb Deletion Cohort

One hundred eighty-one specimens were used in the 3.7 kb deletion evaluation. The mean (SD) age of patients that provided specimens was 34.4 (6.9) years. The mean ages were similar between the control and case groups with mean (SD) ages of 37.2 (6.8) and 33.8 (6.8) years, respectively and were not statistically significantly different (p=0.166). One hundred nineteen (65.7%) patients reported no hormone therapy within the three months prior to specimen collection, 55 (30.4%) reported having hormone therapy within three months prior to specimen collection, and 7 (3.9%) were undetermined. Twenty-six (14.4%) patients reported no menstruation, 15 (8.3%) reported irregular menstruation, 31 (17.1%) were in the menstrual phase (1 to 5 days), 44 (24.3%) were in the follicular phase (5 to 14 days), and 65 (35.9%) were in the luteal+extended menstrual phase (>15 days).

The control group included a total of 32 specimens; 19 (58.4%) specimens collected from symptomatic patients and 13 (40.6%) specimens collected from asymptomatic patients. The test group included 149 specimens. Fifty-two (34.9%) specimens were collected from women with peritoneal, 47 (31.5%) were from women with ovarian, and 50 (33.6%) were collected from women with deep endometriosis. Sixty-five (43.6%) specimens were from women with stage I disease, 24 (16.1%) were stage 11, 30 (20.1%) were stage 111, and 28 (18.8%) were stage IV. Two (1.3%) specimens had an unknown disease stage.

mtDNA Deletions and Preliminary Evaluation—Standard PCR

Seven candidate deletions were initially selected for evaluation based upon sequence composition, presence of a flanking repeat location within the major arc of the mitochondrial genome where proportionally more deletions are reported [47] and observation previously in endometrial tissue (data not shown). Deletions were selected within the following genomic regions: CO2 to ATP6 (1.0 kb deletion); ATP6 to ND3 (1.2 kb deletion); ATP8 to ND4 (2.4 kb deletion); ATP6 to ND5 (3.7 kb deletion); ATP8 to ND5 (5.0 kb deletion); CO1 to ND5 (6.5 kb deletion); and CO2 to CytB (7.7 kb deletion). An initial round of standard (qualitative) PCR and visualization after gel electrophoresis was used to pre-qualify each deletion target and determine if each of the candidates: (i) were detectable; (ii) had sufficient copy number for reliable detection; (iii) had the predicted amplicon size; (iv) were specific and did not co-amplify nuclear pseudogenes or generate non-specific amplification products.

All seven predicted deletions were detectable circulating in blood plasma. However, the 5.0 kb, and the 6.5 kb deletions amplified the rho 0 cell DNA indicating potential co-amplification of nuclear mitochondrial pseudogenes (numts). Additionally, the 6.5 kb deletion had insufficient copy number and was not considered a viable candidate for further QPCR testing. The 7.7 kb and the 2.4 kb deletions were lower in copy number, however still potentially detectable with QPCR so these were subject to further evaluation. The 5.0 kb deletion amplified DNA from the buccal swab of a healthy male indicating a potential lack of disease specificity. The 7.7 kb deletion had a low level of amplification from this specimen as well.

The remaining six deletions were further evaluated using QPCR to determine whether the targets were i) present in sufficient copy number in the absence of whole genome amplification, ii) of sufficient diagnostic accuracy, iii) detectable in rho 0 cells using more sensitive QPCR, and iv) whether the assays' precision was acceptable. Acceptable precision criteria were a maximum deviation of 1.5 Ct between a minimum of two out of three replicates for each target deletion.

Preliminary Evaluation with Clinical Samples

As a preliminary assessment of the remaining six candidates, we evaluated the deletions using a set of 55 clinical specimens; 46 specimens were from patients with confirmed endometriosis and nine specimens were from symptomatic control patients. After initial QPCR testing, the 2.4 kb deletion was determined to have insufficient copy number and the 1.0 kb deletion amplified DNA extracted from rho 0 cells indicating co-amplification of numts and the 7.7 kb deletion amplified only at less stringent annealing temperatures meaning mis-priming events would be more probable. These candidate deletions did not satisfy assay requirements as designed here but may still exist as biomarkers benefiting from further assay optimization to obtain better sequence specificity and assay sensitivity.

Of the seven deletions initially selected, the 1.2 kb and 3.7 kb deletions were present in sufficient copy number in plasma to facilitate easy and reliable detection. The assays were specific under the tested PCR conditions and accurately discriminated between healthy (asymptomatic) control specimens and specimens from confirmed endometriosis patients (data not shown). In addition, both the 1.2 kb and 3.7 kb deletions were also accurate in discriminating between symptomatic controls and endometrial disease cases (all subtypes and stages combined). The AUC (95% CI) for the 1.2 kb deletion was 0.8116 (0.6178-1.005), which was statistically significant (p=0.0034). Similarly, the AUC (95% CI) for the 3.7 kb deletion was 0.8478 (0.6663-1.029), which was also significant (p=0.0011; Table 11).

TABLE 11
Preliminary Evaluation of the deletions

| | Standard PCR + visualization | | | | | Quantitative real-time PCR (N = 55) | | |
|---|---|---|---|---|---|---|---|---|
| Deletion | | | | Target | | | Diagnostic | |
| ID | Detect- | Sufficient | Target | specificity | | Sufficient | accuracy | |
| (Deletion | able in | copy | specificity | (amplicon | Disease | copy | AUC (95% | |
| size) | plasma | number | (rho 0) | size) | specificity | number | Cl) | p-value |
| 1 | Yes | Yes | No | Yes | No | Yes | 0.82 | |
| (5.0 kb) | | | | | | | (0.64- | |
| | | | | | | | 1.00) | |
| 4 | Yes | Low | Possible | Yes | Possible | Yes | 0.84 | |
| (7.7 kb) | | | | | | | | |
| 14a | Yes | Yes | Yes | Yes | Yes | Yes | 0.85 | 0.0011 |
| (3.7 kb) | | | | | | | (0.6663- | |
| | | | | | | | 1.029) | |
| 120 | Yes | No | No | No | N/A | N/A | | |
| (6.5 kb) | | | | | | | | |
| 122 | Yes | Yes | Yes | Yes | Yes | Yes | 0.83 | |
| (1.0 kb) | | | | | | | (0.64-1.01) | |
| 193 | Yes | Yes | Yes | Yes | Yes | Yes | 0.81 | 0.0034 |
| (1.2 kb) | | | | | | | (0.6178- | |
| | | | | | | | 1.005) | |
| 586 | Yes | Low | Yes | Possible | Yes | No | 0.82 | |
| (2.4 kb) | | | | | | | (0.66-0.97) | |

Abbreviations: AUC = area under the curve; CI = confidence interval; N = number of specimens in evaluation set; PCR = polymerase chain reaction.

### Diagnostic Accuracy of the 1.2 kb and 3.7 kb Deletions

To more fully evaluate the 1.2 kb and 3.7 kb deletions as clinically viable biomarkers of endometriosis, we determined the ability of these deletions to discriminate between symptomatic controls and all endometriosis types combined, between three subtypes, and four stages of endometriosis using a larger set of clinical specimens (Table 10). Valid paired results (both target and 18S gene amplification) were obtained for 171 specimens with the 1.2 kb deletion and 181 specimens with the 3.7 kb deletion. These analyses were performed using only symptomatic control and confirmed disease specimens in order to more accurately reflect the clinically relevant patient populations—that is, women presenting with symptoms of endometriosis with surgical confirmation of disease status as an outcome. Importantly, the 1.2 kb and 3.7 kb deletions detected no difference between symptomatic and asymptomatic control specimens, p=0.462 and p=0.878, respectively.

### Symptomatic Controls Vs all Disease

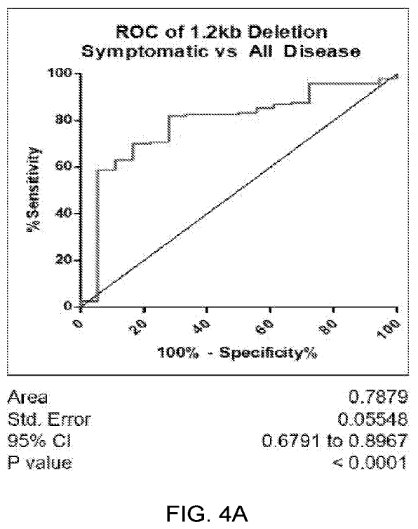

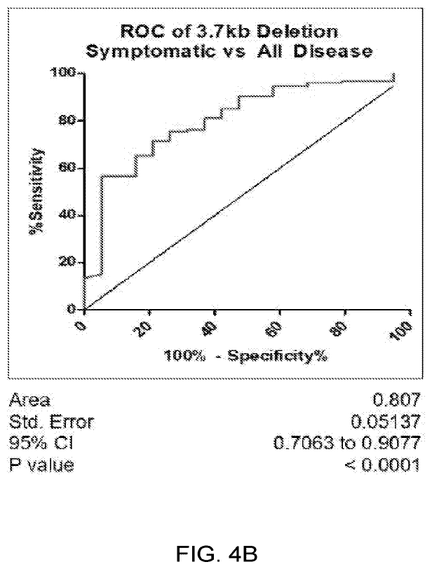

Similar to the preliminary analysis using 55 clinical specimens, both the 1.2 kb and 3.7 kb deletions accurately discriminated between symptomatic control and endometrial disease specimens (peritoneal, ovarian, and deep endometriosis specimens combined). The AUC (95% CI) for the 1.2 kb deletion was 0.7879 (0.6791-0.8967), which was statistically significant (p<0.0001). The AUC (95% CI) for the 3.7 kb deletion was 0.807 (0.7063-0.9077), which was also significant (p<0.0001; FIGS. 4A and 4B). Coordinates of the receiver operating (ROC) curve were examined and a threshold, or cut-off, selected to optimize sensitivity. Applying a threshold of −4.43 for discrimination of symptomatic controls and all subtypes/stages of endometriosis using the 1.2 kb deletion results in sensitivity and specificity values of 81.8% and 72.2%, respectively. At a threshold of 10.51, sensitivity and specificity for the 3.7 kb deletion are 85.1% and 57.9%, respectively (Table 12).

TABLE 12
Performance of the 1.2 kb and 3.7 kb deletions

| | 1.2 kb deletion | | 3.7 kb deletion | |
|---|---|---|---|---|
| | Sensitivity | Specificity | Sensitivity | Specificity |
| | (%) | (%) | (%) | (%) |
| All disease | 81.8 | 72.2 | 85.0 | 57.9 |
| Peritoneal | 91.8 | 72.2 | 88.5 | 73.7 |
| Ovarian | 75.6 | 72.2 | 80.9 | 68.4 |
| Deep | 78.6 | 66.7 | 80.0 | 52.6 |
| infiltrating | | | | |
| Stage I/II | 82.1 | 72.2 | 87.6 | 63.2 |
| Stage III/IV | 80.7 | 72.2 | 84.5 | 52.6 |

Combining the 1.2 kb deletion with the 3.7 kb deletion improved diagnostic accuracy (AUC 0.827 (0.722-0.931) between all symptomatic controls and all endometriosis, and AUC 0.882 (0.784-0.980) between symptomatic controls and stage I/II disease (data not shown).

### Disease by Subtype—1.2 kb Deletion

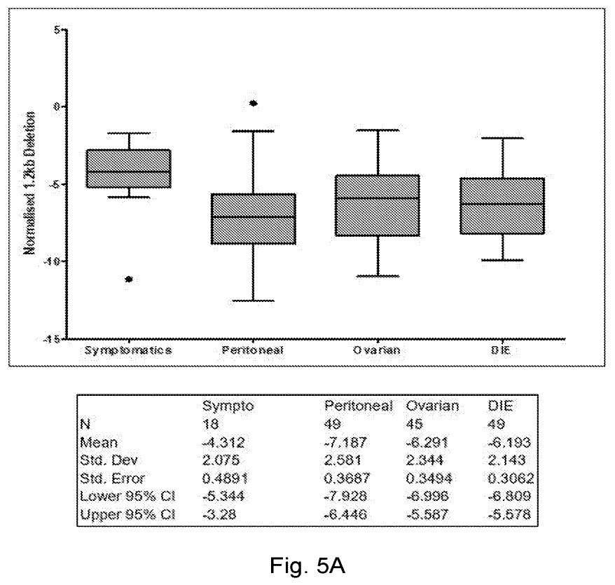

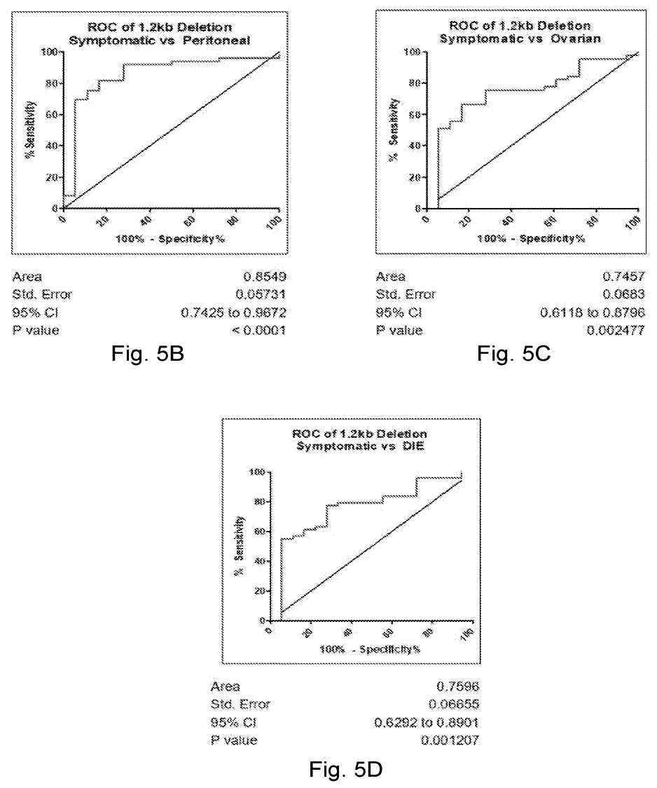

An important feature of any diagnostic aid for endometriosis is the ability to accurately detect all disease subtypes. We evaluated the ability of the 1.2 kb deletion to differentiate between symptomatic control specimens and specimens from patients with confirmed peritoneal, ovarian, and deep endometriosis. The distribution of the 1.2 kb deletion for each disease subtype is shown in FIG. 5A. The mean (SD) ΔCt value was −4.312 (2.075), for symptomatic controls, −7.187 (2.581) for peritoneal disease, −6.291 (2.344), for ovarian disease, and −6.193 (2.143), for deep endometriosis. The difference in normalized 1.2 kb deletion quantity between symptomatic controls was statistically significant for peritoneal (p<0.0001), ovarian (p=0.003), and deep endometriosis (p=0.0012).

Diagnostic accuracy of the 1.2 kb deletion is shown in FIGS. 5B to SD, with AUC (95% CI) values of 0.8549 (0.7425-0.9672), p<0.0001 for detection of peritoneal, 0.7457 (0.6118-0.8796), p=0.0025 for detection of ovarian, and 0.7596 (0.6292-0.8901), p=0.0012 for detection of deep endometriosis. Taken together, these data indicate that the 1.2 kb deletion was able to accurately distinguish between specimens collected from symptomatic controls and peritoneal, ovarian, and deep endometriosis patients. Applying a threshold of −4.430 for discrimination of symptomatic controls and peritoneal endometriosis using the 1.2 kb deletion results in sensitivity and specificity values of 81.8% and 72.2%, respectively. At a threshold of −4.675, the sensitivity and specificity of the 1.2 kb deletion in discriminating between symptomatic controls and ovarian endometriosis is 75.6% and 72.2%, respectively. At a threshold of −4.350, the sensitivity and specificity of the 1.2 kb deletion in discriminating between symptomatic controls and deep endometriosis is 78.6% and 66.7%, respectively (Table 12).

### Disease by Subtype—3.7 kb Deletion

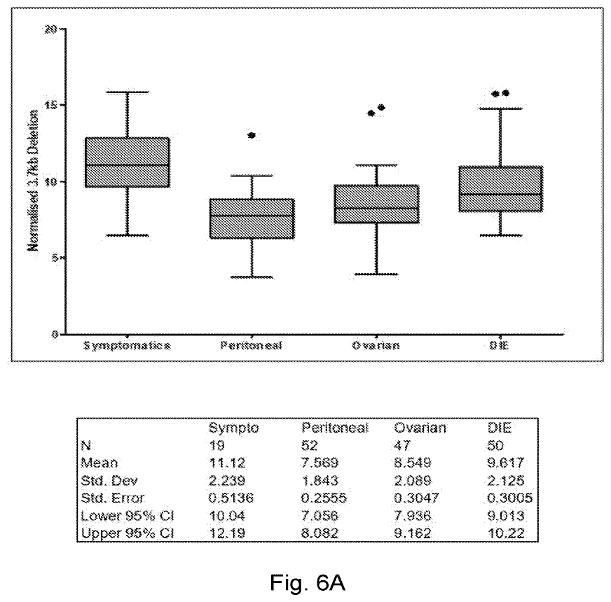

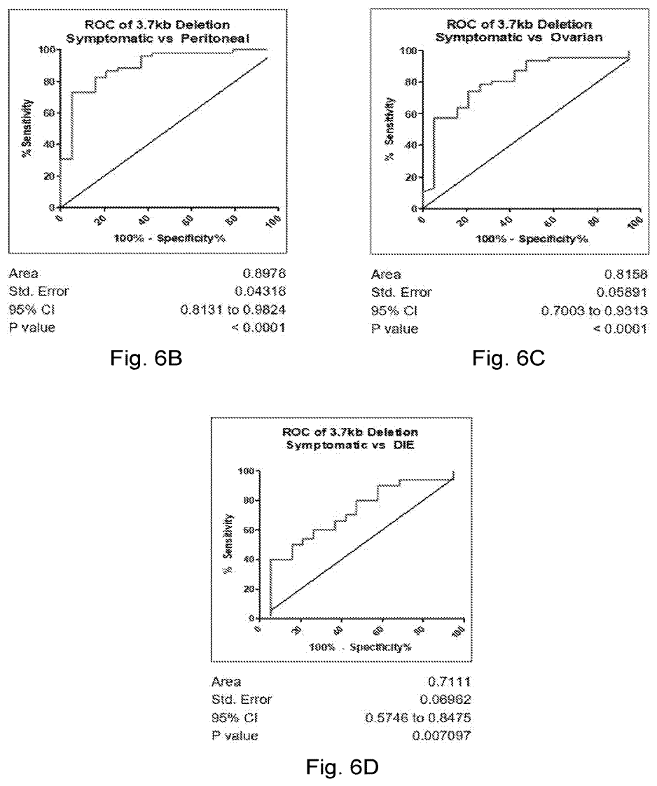

The distribution of the 3.7 kb deletion for each disease subtype is shown in FIG. 6A. The mean (SD) ΔCt value was 11.12 (2.239), for symptomatic controls, 7.569 (1.843) for peritoneal, 8.549 (2.089), for ovarian, and 8.617 (2.125) for deep endometriosis. The difference in amplicon quantity between symptomatic controls was statistically significant for peritoneal (p<0.0001), ovarian (p<0.0001), and deep endometriosis (p=0.0072). Diagnostic accuracy of the 3.7 kb deletion in detecting each of the three disease subtypes is shown in FIGS. 6B to 6D, with AUC (95% CI) values of 0.8978 (0.8131-0.9824), p<0.0001 for detection of peritoneal, 0.8158 (0.7003-0.9313), p<0.0001 for detection of ovarian, and 0.7110 (0.5746-0.8475), p=0.0071 for detection of deep endometriosis. Taken together, these data indicate that the 3.7 kb deletion was able to accurately distinguish between specimens collected from symptomatic controls and women with peritoneal, ovarian, and deep endometriosis. Applying a threshold of 8.805 for discrimination of symptomatic controls and peritoneal endometriosis using the 3.7 kb deletion results in sensitivity and specificity values of 88.5% and 73.7%, respectively. At a threshold of 8.910, the sensitivity and specificity of the 3.7 kb deletion in discriminating between symptomatic controls and ovarian endometriosis is 80.9% and 68.4%, respectively. At a threshold of 11.01, the sensitivity and specificity of the 3.7 kb deletion in discriminating between symptomatic controls and deep endometriosis is 80.0% and 52.6%, respectively (Table 12).

### Disease by Stage—1.2 kb Deletion

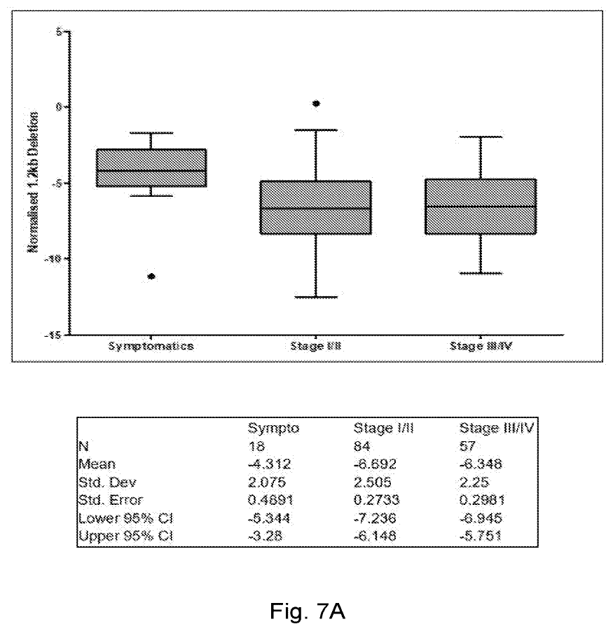

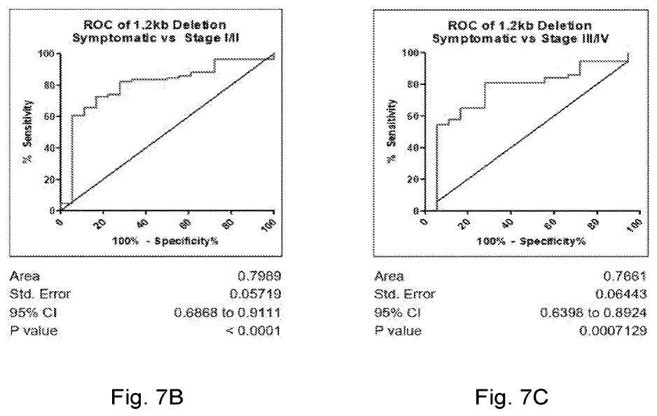

Another important characteristic of a biomarker for endometriosis is the ability to detect both low and high stages of disease. We next evaluated the ability of the 1.2 kb deletion to differentiate between symptomatic control specimens and specimens from confirmed low (I/II) or high (III/IV) stages of disease. The distribution of the 1.2 kb deletion for stage I/II and stage III/IV disease is shown in FIG. 7A. The mean (SD) ΔCt value was −4.312 (2.075), for symptomatic controls, −6.692 (2.505) for stage I/II, and −6.348 (2.25), for stage III/IV. The difference between symptomatic controls was statistically significant for stage I/II (p<0.0001), and stage III/IV (p=0.001) disease groups. The difference between stage I/II and III/IV was not statistically significant (p=0.406).

Diagnostic accuracy of the 1.2 kb deletion is shown in FIGS. 7A to 7C, with AUC (95% CI) values of 0.7989 (0.6868-0.9111), p<0.0001 for detection of stage I/II, and 0.7661 (0.6398-0.8924), p=0.0007 for detection of stage III/IV disease. Thus, the 1.2 kb deletion was able to accurately distinguish between symptomatic controls and all stages of disease. At a threshold of −4.430, the sensitivity and specificity of the 1.2 kb deletion in discriminating between symptomatic controls and stage I/II endometriosis is 82.1% and 72.2%, respectively. At a threshold of −4.490, the sensitivity and specificity of the 1.2 kb deletion in discriminating between symptomatic controls and stage III/IV endometriosis is 80.7% and 72.2%, respectively (Table 12).

### Disease by Stage—3.7 kb Deletion

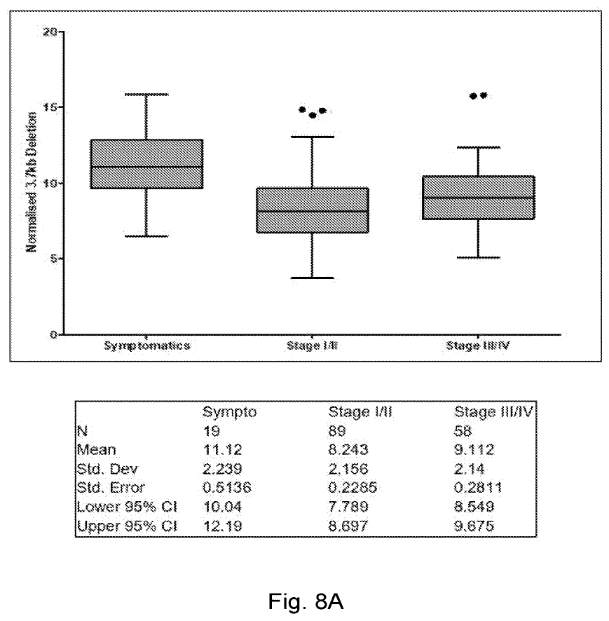

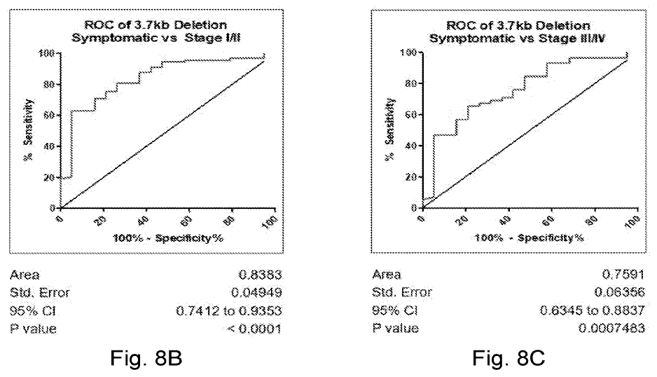

The distribution of the 3.7 kb deletion for stage I/II and stage III/IV disease is shown in FIG. 8A. The mean (SD) ΔCt value was 11.12 (2.239), for symptomatic controls, 8.243 (2.156) for stage I/II, and 8.112 (2.14) for stage III/IV. The difference between symptomatic controls was statistically significant for stage I/II (p<0.0001), and stage III/IV (p=0.0008) disease groups. Diagnostic accuracy of the 3.7 kb deletion is shown in FIGS. 8B to 8C, with AUC (95% CI) values of 0.8383 (0.7412-0.9353), p<0.0001 for detection of stage I/II, and 0.7591 (0.6354-0.8837), p=0.0007 for detection of stage III/IV disease. The difference between stage I/II and III/IV was statistically significant for the 3.7 kb deletion (p=0.016). These data indicate that the 3.7 kb deletion was able to accurately distinguish between symptomatic controls and all stages of disease. At a threshold of 10.17, the sensitivity and specificity of the 3.7 kb deletion in discriminating between symptomatic controls and stage I/II endometriosis is 87.6% and 63.2%, respectively. At a threshold of 11.00, the sensitivity and specificity of the 3.7 kb deletion in discriminating between symptomatic controls and stage III/IV endometriosis is 84.5% and 52.6%, respectively.

Correlation with Patient Age, Specimen Age, Hormonal Therapy, and Menstrual Phase—1.2 kb Deletion

An ideal biomarker test would provide accurate results independent of patient and specimen age, treatment with hormonal therapy, and timing of menstrual phase during specimen collection. The effect of these parameters on disease detection is summarized in Table 13.

TABLE 13
Effect of patient and
specimen age, hormonal therapy, and menstrual cycle

| | | | | | T-test⁽¹⁾ or | |
|---|---|---|---|---|---|---|
| | Pearson's | | | | ANOVA⁽²⁾ | |
| | correlation (r) | | p value | | p value | |
| Parameter | 1.2 kb | 3.7 kb | 1.2 kb | 3.7 kb | 1.2 kb | 3.7 kb |
| Patient age | 0.030 | 0.1034 | 0.698 | 0.166 | — | |
| Specimen age | 0.072 | 0.0628 | 0.353 | 0.4009 | — | |
| Hormonal status⁽¹⁾ | — | | — | | 0.120 | 0.195 |
| Menstrual cycle⁽²⁾ | — | | — | | 0.228 | 0.036 |

⁽¹⁾T-test was used to determine effect of hormonal status on detection of endometriosis
⁽²⁾ANOVA was used to determine effect of menstrual cycle on detection of endometriosis

For the 1.2 kb deletion, we determined that there was no correlation with the detection of disease and patient age; the correlation coefficient (r) was r=0.030 (p=0.698). There was also no correlation with disease detection and specimen age by year of collection (r=0.072, p=0.353). When stratified by hormonal status (patients that received hormonal therapy or had no hormonal therapy within the 3 months prior to specimen collection) the difference in disease detection was not statistically significant (p=0.120). When patients were stratified by menstrual phase (no menstruation, irregular menstruation, menstruation, follicular phase, or luteal phase+extended menstruation) there was no statistically significant difference in disease detection with the 1.2 kb deletion (p=0.228).

Similarly, disease detection based on the 3.7 kb deletion was not significantly correlated to patient age (r=0.1034; p=0.166) or specimen age (r=0.0628; p=0.4009) or significantly affected by hormonal therapy (p=0.195). Detection of endometriosis with the 3.7 kb deletion was significantly correlated to menstrual phase (p=0.036), which was driven specifically by the difference in detection between patients who reported no period and those that were in the follicular phase (p=0.026) with a difference of 1.72. Combined, these data indicate that the accuracy of the 1.2 kb and is not significantly affected by these clinically relevant variables and accuracy of the 3.7 kb deletion is slightly affected by menstrual phase.

### Discussion

Endometriosis is a highly prevalent disease in women of reproductive age that is associated with a large economic burden and results in a substantial reduction in the quality of life of those affected. One of the key contributors to this clinical problem is the lack of diagnostic tools to facilitate early detection and intervention. The current diagnostic gold standard is a thorough laparoscopic inspection ideally followed by histologic confirmation of suspected lesions [5, 15]. Because there are minimal objective data available, the diagnostic value of this process in largely unclear. The use of laparoscopic exams has been considered by some to be potentially inaccurate, and even paired with histologic confirmation accuracy reportedly ranges from 60% to 85% [48-51]. The standard diagnostic process could be further complicated in cases where the disease presents itself atypically, or in early stages of disease that are not easily visualized and overlooked by inexperienced surgeons. Additionally, where medical intervention could be initiated in an effort to avoid or delay surgery, a presumptive diagnosis of endometriosis based upon an accurate biomarker test could provide needed evidence in support of this treatment. Thus, there is a clear need to improve upon the current standard, particularly in a way that could provide more routine results early in the course of disease.

In the current study, we identified and evaluated two novel mtDNA deletions as potential biomarkers for endometriosis. Assays targeting the 1.2 kb and 3.7 kb deletions met criteria for robust diagnostic tests, utilize a minimally-invasive specimen, and if successfully translated into clinical use, could potentially help reduce the delay in time to diagnosis associated with current diagnostic practices and provide an opportunity for medical intervention prior to a surgical one. After setting a diagnostic threshold (as described above), the sensitivity of the 1.2 kb deletion assay was 81.8% and specificity was 72.2%. The diagnostic performance of the 3.7 kb deletion assay was similar with sensitivity and specificity of 85.1% and 57.9%, respectively. Thus, diagnostic assays based on either of these deletions have the potential to compliment the current standard of care. Of particular importance is the diagnostic accuracy of these deletions for early stage disease as later stage disease is more readily detected in current practice using ultrasound. In a primary care setting a positive test result could support initiating first-line medical treatment for endometriosis such as oral contraceptives or trigger a specialist referral. An estimated 10% of women presenting with dysmenorrhea have secondary dysmenorrhea, with the majority caused by endometriosis [52]. In this population the 1.2 kb and 3.7 kb deletions would quite effectively rule out endometriosis with a negative predictive value (NPV) of 97%. In a secondary care setting, a positive test could guide the decision to initiate treatment with second-line medication such as gonadotropin-releasing hormone antagonists or to proceed with laparoscopic surgery. Importantly, in the former setting a diagnostic cut-off could be selected to maximize test sensitivity as the risk associated with an incorrect false positive result is less critical, whereas in the latter setting a different diagnostic cut-off to maximize specificity and minimize the exposure of women without the disease to the risks associated with these interventions could be beneficial.

In addition to diagnostic accuracy, the ability of these two biomarkers to detect endometriosis was not correlated to patient age, specimen age, or hormone status, and only the 3.7 kb deletion had a slight correlation to the patients' phase of menstrual cycle at the time of specimen collection. Further study is needed to confirm whether this correlation with menstrual phase exists in a larger cohort of patients, or whether it is an artifact of the number of specimens used in this study. We demonstrated that both the 1.2 kb and 3.7 kb deletions accurately detect all subtypes and stages of disease. In contrast to the current diagnostic standard that involves visualization during surgery and possible excision of lesions for histological confirmation, assays based on mtDNA deletions require only a blood specimen and could potentially provide objective results before or instead of a surgical intervention. Thus, if successfully translated into clinical use, mtDNA-based assays have the potential to reduce the delays in diagnosis [16] and provide actionable results earlier in the course of disease than currently possible.

From a practical standpoint, use of a blood-based biomarker assay has several advantages to effectively augment the current standard of care. The specimen is easy and inexpensive to collect via venipuncture, and there is a low likelihood to have co-morbidities associated with collection. Blood specimens can be readily collected in an outpatient physicians' office or clinic, eliminating the need for dedicated surgical space and equipment. As a result of the high copy number of mtDNA, standard DNA extraction methods are used without the need for enrichment techniques and ample DNA is recovered from a standard blood specimen so a low test failure rate can be anticipated. The assays use PCR-based technology that is cost-effective and widely used in clinical labs, and while the assays are quantitative, the output is easily interpreted—that is, a test result is either above or below a defined diagnostic cut-off which corresponds to either a positive or negative outcome. Finally, a lack of, or minimal correlation with menstrual stage ensures that sampling requirements are simplified, and timing of menstruation need not be considered when scheduling venipuncture.

A key element in successful disease management is understanding disease epidemiology. Due in part to a relatively complex diagnostic process and symptoms that overlap with other gynecological disorders, the epidemiology of endometriosis is not well-characterized and varies across patient populations and geographic locations [1, 2, 4, 6]. With the advent of molecular assays such as those described here, additional data could become more readily available and help fill in some of the gaps in our understanding of endometriosis epidemiology. Importantly, this study utilized publicly available standardized processes for specimen collection and processing, which will allow for more direct comparison of test results across different studies and patient populations [41-44, 53].

Importantly, the location of the mtDNA deletions may also help shed light on the pathophysiological process of endometriosis. Both the 1.2 kb and 3.7 kb deletions affect all or part of the genes encoding for Complexes I and V (ATP synthase) of the respiratory chain and several tRNAs. Although these deletions likely result in abnormal mitochondrial ATP synthase and Complex I proteins, the heteroplasmic nature of mtDNA likely allows some degree of functional compensation within the population. Interestingly, the two best candidates out of the seven tested in this study are deletions within regions that overlap each other in the mitochondrial genome. Given that the 1.2 kb deletion region (ATP6 to ND3) resides within the larger 3.7 kb deletion (ATP6 to ND5), perhaps it is not surprising that the diagnostic accuracy of the two deletions is similar.

Based on the data presented here, the 1.2 kb and 3.7 kb mtDNA deletions are associated with endometriosis; however, additional study is necessary to understand what mechanistic role this mitochondrial genomic region plays in the development of endometriosis.

Limitations of this study include the use of patient reported hormone and menstrual status, which can be less accurate than taking study-specific data measurements. This data, while encouraging, requires replication and validation in a larger, independent data set. This study is currently underway.

### SUMMARY

The following is a summary of the study discussed above:
Endometriosis is a significant health burden that affects up to 10% of women worldwide. Currently, diagnosis is based on surgical visualization followed by histological confirmation.
Diagnosis is often complicated due to variable clinical presentation and symptoms that overlap with other gynecological disorders. As a result, definitive diagnosis can be delayed up to a decade, which can result in higher morbidity and decreased quality of life for those affected.
Thus, there is a clear need for rapid, reliable diagnostic aids that can provide actionable results early in the course of disease.
Study specimens were collected from women scheduled to undergo laparoscopy for pelvic pain (symptomatic) or tubal ligation (asymptomatic). Study participants were female, aged 18 years or older (until menopause), and were confirmed as not pregnant.
Seven candidate mtDNA deletions were identified and evaluated to determine whether each was detectable in plasma, had sufficient copy number for reliable detection, had the predicted amplicon size, were specific and did not co amplify nuclear pseudogenes or generate non-specific amplification products.
Six candidate deletions were further evaluated by QPCR and clinical specimens to determine whether each met the criteria for a robust diagnostic assay and evaluated accuracy in discriminating between endometriosis and control specimens. Two deletions were selected as potential biomarker candidates (1.2 kb and 3.7 kb deletions).
The 1.2 kb and 3.7 kb deletions accurately detected endometriosis, including all subtypes and disease stages, and detection was not correlated to patient or specimen age or hormone therapy. The 3.7 kb deletion was significantly correlated to menstrual phase, which was limited only to two phases.
Biomarkers derived from the mitochondrial genome, including the 1.2 kb and 3.7 kb deletions described here, offer a promising and largely unexplored avenue in the pursuit of diagnostic markers for endometriosis that can be effectively translated to clinical application.
Based on a minimally invasive specimen, assays based on these markers could positively impact the diagnostic landscape for endometriosis by reducing the delay in diagnosis and providing rapid, actionable, and objective test results.

### Conclusion

Biomarkers derived from the mitochondrial genome, in particular the 1.2 kb and 3.7 kb deletions described here, offer a promising and largely unexplored avenue in the pursuit of diagnostic markers for endometriosis that can be effectively translated to clinical application. Based on a minimally invasive specimen, assays based on these markers have been found to accurately diagnose endometriosis in blood samples from patients. Thus, the present description provides a rapid, accurate, and efficient means of diagnosing endometriosis thereby resulting in a reduction in the delay in obtaining a diagnosis and administering the necessary treatment protocol. The present description allows for the subject diagnosis to be performed on one or more of the mtDNA deletion (including either the large or small sublimon) and any fusion transcripts resulting therefrom. The same conclusion may also be extended to any translation products resulting from the fusion transcripts.

### Example 3: Identification of 8.7 kb mtDNA Deletion for Detecting Endometriosis

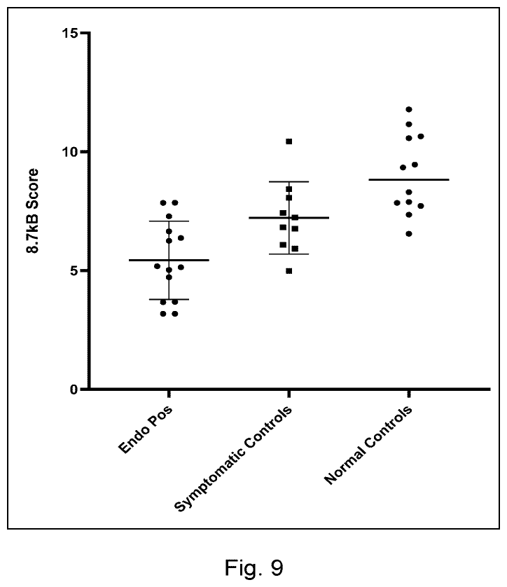

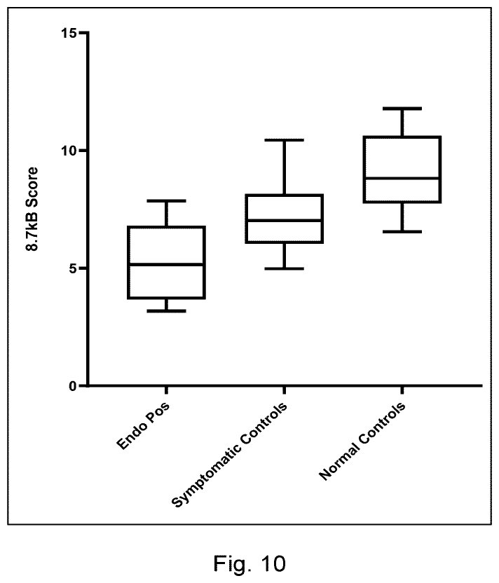

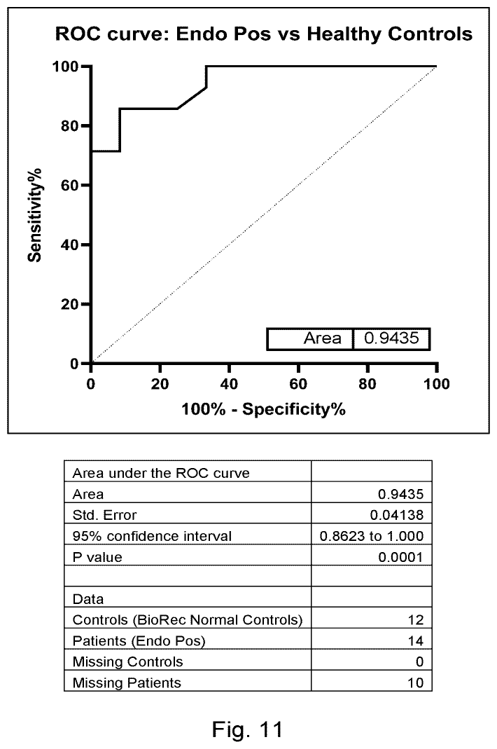

In this study, we identified the 8.7 kb deletion (Deletion ID No. 2767) using a combination of next generation sequencing (NGS) and proprietary data mining software in a set of 10 cases and 10 controls obtained from Fidelis Research (Sofia, Bulgaria). The methodology used for this identification is described in more detail in Example 4. We detected this biomarker directly in endometriosis tissue lesions using both qPCR and NGS. We selected the 8.7 kb deletion for evaluation based upon sequence composition, the presence of a flanking repeat location within the major arc of the mitochondrial genome where proportionally more deletions are reported [47] and observation in endometrial tissue. The data from this study is provided in Table 14 and illustrated in FIGS. 9 to 11.

TABLE 14
Identification of 8.7 kb mtDNA deletion

| | Endometriosis | Symptomatic | Normal/Healthy |
|---|---|---|---|
| | Positive | Controls | Controls |
| Number of values | 14 | 10 | 12 |
| Minimum | 3.18 | 4.985 | 6.55 |
| 25% Percentile | 3.678 | 6.04 | 7.753 |
| Median | 5.158 | 7.028 | 8.82 |
| 75% Percentile | 6.808 | 8.156 | 10.63 |
| Maximum | 7.86 | 10.44 | 11.78 |
| Range | 4.68 | 5.455 | 5.23 |
| Mean | 5.431 | 7.216 | 9.052 |
| Std. Deviation | 1.648 | 1.526 | 1.686 |
| Std. Error of Mean | 0.4406 | 0.4824 | 0.4868 |
| Lower 95% CI of | 4.479 | 6.125 | 7.98 |
| mean | | | |
| Upper 95% CI of | 6.383 | 8.307 | 10.12 |
| mean | | | |
| Sum | 76.04 | 72.16 | 108.6 |

The 8.7 kb deletion removes all or part of the genes between NADH dehydrogenase subunits 2-5. An initial round of standard (qualitative) PCR and visualization after gel electrophoresis was used to pre-qualify the deletion target and determine if the deletion: (i) was detectable; (ii) had sufficient copy number for reliable detection; (iii) had the predicted amplicon size; (iv) was specific and did not co-amplify nuclear pseudogenes or generate non-specific amplification products.

We successfully detected the deletion in circulating plasma and performed further evaluation by qPCR to determine whether the target was detectable in rho 0 cells using more sensitive qPCR. We also evaluated if the assay had sufficient diagnostic accuracy and acceptable precision (defined as a maximum deviation of 1.5 Ct between at least two of three replicates).

Further investigation of this deletion is described in Example 4.

### Example 4: 8.7 kb mtDNA Deletion for Detecting Endometriosis in Plasma of Symptomatic Women

In this example, the 8.7 kb mtDNA deletion (FUS 5362:14049) was investigated as a potential biomarker for diagnosing endometriosis, including i) an initial assessment of diagnostic accuracy followed by ii) an evaluation of disease specificity by comparing the biomarker's frequency in plasma from women with: endometriosis and symptomatic controls, and endometrial cancer, ovarian cancer, and breast cancer.

### Methods

### Diagnostic Accuracy—Participants and Sample Collection

This was a case control study in which residual plasma samples prospectively collected from women aged 18 years and over (until menopause) who were not pregnant and were scheduled to undergo laparoscopy for suspected endometriosis because of pelvic pain (symptomatic controls and endometriosis cases) or tubal ligation (asymptomatic controls) were used. This study was conducted as part of the EndOx study at Oxford Endometriosis CaRe Centre, John Radcliffe Hospital, University of Oxford, UK

The collection, anonymization and processing of samples and data were as previously reported [26, 41-44, 57]. Study conduct, relevant authority approvals (Oxfordshire REC A, 09/H0604/58) and consent procedures were also as previously reported [26, 41-44; 57].

### Diagnostic Accuracy—Participant Populations/Cohorts

Collected samples were classified as either control or case samples. The control group comprised a) asymptomatic controls, which were specimens collected from participants who underwent scheduled tubal ligation without clinical suspicion of endometriosis, and who had surgically confirmed absence of endometriosis; and b) symptomatic controls, which were collected from participants with pain or other symptoms (excluding infertility) with a clinical suspicion of endometriosis, but no endometriosis lesions visualized by laparoscopy by experienced gynecological surgeons.

The case group comprised specimens for which the presence of endometriosis was diagnosed during laparoscopy and classified by the operating surgeon using the revised American Society of Reproductive Medicine (rASRM) stages (I: minimal; II: mild; Ill: moderate; IV: severe disease) [45]. Specimens were also grouped by disease subtype: peritoneal, ovarian, and deep infiltrating (DI) endometriosis.

### Disease Specificity—Participants and Sample Collection

Endometriosis cases and controls from the diagnostic accuracy assessment were utilized for the assessment of disease specificity and compared to residual plasma samples obtained from OBIO (El Segundo, USA) and Ontario Tumour Bank (Toronto, Canada).

Sample Handling, Processing, and mtDNA Amplification

### Blood Collection and Processing

Whole blood was collected in 10 ml K2EDTA Vacutainers® (BD Medical p/n BD366643) and centrifuged within 1 hour of collection at 2500×g for 10 minutes at 4 °C. The plasma layer was removed, aliquoted and stored at −80 °C until DNA extraction.

### DNA Extraction

Total deoxyribonucleic (DNA) was extracted from blood plasma (200 μL) using the QIAamp™ 96 QIAcube™ HT extraction kit (Qiagen, Crawley, UK), automated on a QIAcube™ HT system (Qiagen, Crawley, UK), and eluted extracted DNA with buffer AE (200 μL).

mtDNA Deletion qPCR and qPCR Normalization with 18s rRNA

For both real time polymerase chain reaction (qPCR) procedures, we performed amplification in 20 μL reactions using a 96 well microplate (Bio-Rad, Hemel Hempstead, UK), with each well containing un normalized DNA template (5 μL), SYBR® Green master mix and 250 nM of each primer for the 8.7 kb deletion and the 18S ribosomal ribonucleic acid (rRNA). The primers used are provided in Table 15.

TABLE 15
Primer sequences used for amplification of 8.7 kb deletion

| | Primer | | | |
|---|---|---|---|---|
| Deletion ID | Direction | Length | Sequence | SEQ ID NO. |
| 2767 | Fwd | 25 | GGGCCATTATCGAAGAATTCACAAA | 82 |
| (“8.7 kb | Rev | 24 | GAGGTGATGATGGAGGTGGAGTAG | 83 |
| Deletion”) | (junction | | | |
| | primer) | | | |

We used a CFX96 Touch Real time PCR Detection System (Bio-Rad, Hemel Hempstead, UK) for quantitative polymerase chain reaction (QPCR) with SYBR Green 1 fluorescence.

Cycling conditions for the 8.7 kb deletion and 18S rRNA were: 45 cycles of 30 seconds at 95 °C, 30 seconds at 66 °C, and 30 seconds at 72 °C. After amplification, we performed melting curve analysis from 70 °C to 90 °C, with a reading every 0.5 °C. Each plate of samples and controls was amplified in triplicate on three separate occasions.

### Quality Control

Quality control was performed as previously described [56]. In brief, we calculated the quantification cycle (Cq) and normalized the Cq of the deletion amplicon to the Cq of the multi-copy nuclear target 18s rRNA gene amplicon. We amplified all samples in triplicate on separate plates. Two no-template control samples were processed alongside each batch of DNA extractions and verified as negative for amplification of both the deletion target and the 18s rRNA gene.

Rho 0 cell Preparation

Rho 0 cells were prepared as previously described [46; Creed 2019]. In brief, cells from the human osteosarcoma cell line 143B (ATCC CRL 8303) were treated with ethidium bromide to deplete cytoplasmic mtDNA. Cells were grown to confluence in high glucose Dulbecco's Modified Eagle's Medium with pyruvate, L glutamine, uridine (50 μg/mL) and 5% fetal bovine serum.

### Statistical Analysis

No formal sample size calculation was performed; the number of clinical specimens used was deemed sufficient to satisfy the study objective. For qPCR, targets were amplified from all specimens in triplicate and average Cq values were calculated. We determined the normalized deletion value (ΔCq) by quantifying the deletion amplicon relative to the 18s rRNA reference amplicon. Statistical analyses were performed using Graphpad Prism™ 5.0, (Graphpad Software Inc., La Jolla, CA, USA) for ROCs, descriptive statistics, correlations and significance tests. We summarized clinical characteristics using count and percentages for categorical data, and mean, standard deviation (SD), and range for continuous variables. The means of two groups were compared using the Student's t-test and the Mann-Whitney U test for parametric and non-parametric distributions, respectively. Correlation between the two variables was assessed with the Spearman correlation (r) or the Mann Whitney U-Test or Kruskal-Wallis test. With respect to the presence of endometriosis, ROC curves were constructed. The area under the curve (AUC) of the ROC and the sensitivity and specificity at a selected cut-off was calculated with 95% confidence intervals (CIs). A p value <0.05 was considered statistically significant for all tests.

### Results

### Study Population and Clinical Specimens

Demographics and clinical characteristics for participants who provided specimens are summarized in Table 16.

TABLE 16
Study Population Demographic and Clinical Characteristics

| Characteristic | Total N (%) | Controls N (%) | Cases N (%) |
|---|---|---|---|
| N | 182 | 32 | 150 |
| Patient age¹ | 34.4 (±6.9) | 37.16 (±6.901) | 33.78 (±6.820) |
| Hormone | | | |
| therapy status² | | | |
| Yes | 55 (30.2) | 11 (34.5) | 44 (29.3) |
| No | 120 (65.9) | 19 (59.4) | 101 (67.3) |
| Undetermined | 7 (3.8) | 2 (6.3) | 5 (3.3) |
| Menstrual phase³ | | | |
| No menstruation | 27 (14.8) | 8 (25.0) | 19 (12.7) |
| Irregular menstruation | 15 (8.2) | 2 (6.3) | 13 (8.7) |
| Menstruation | 30 (16.5) | 6 (18.8) | 24 (16.0) |
| Follicular phase | 46 (25.3) | 3 (9.4) | 43 (28.7) |
| Luteal phase and | 64 (35.2) | 13 (40.6) | 51 (34.0) |
| extended | | | |
| Non- | | | |
| Endometriosis/ | | | |
| Endometriosis type | | | |
| Symptomatic | 18 (9.9) | 18 (56.3) | NA |
| Asymptomatic | 14 (7.7) | 14 (43.8) | NA |
| Peritoneal | 52 (28.6) | NA | 52 (34.7) |
| Ovarian | 48 (26.4) | NA | 48 (32.0) |
| Deep infiltrating | 50 (27.5) | NA | 50 (33.3) |
| Endometriosis stage | | | |
| Stage I/II | NA | NA | 91 (60.7) |
| Stage III/IV | NA | NA | 58 (38.7) |
| Unknown | NA | NA | 1 (0.7) |

Abbreviations:
N = number of participants/specimens;
SD = standard deviation.
¹Mean (SD) is presented; mean and SD were collected for participants that provided age at time of specimen collection.
²Participants' status within 3 months of specimen collection.
³Participants' menstrual status at time of specimen collection.

Overall, the mean (SD) ages of the control and case groups were statistically significantly different: 37.2 (6.9) and 33.8 (6.8) years, p=0.0124. The majority of participants (121; 66.5%) were not undergoing hormone therapy within the three months before specimen collection. Most participants who reported no menstruation were on hormones (20/26; 76.9%).

Of the 182 specimens collected, 32 were from the control group, with 18 (9.49%) from symptomatic participants and 14 (7.7%) from asymptomatic participants. The remaining 150 specimens were from the case group in which 52 (28.6%) participants had peritoneal, 48 (26.4%) had ovarian and 50 (27.5%) had DI endometriosis and were classified as 91 (60.7%) with rASRM stage I/II disease and 58 (31.9%) with stage III/IV. Of the 182 specimens 178 (97.8%) produced valid assay results, with 2 peritoneal and 1 ovarian endometriosis samples, and 1 symptomatic control sample invalid for statistical analysis due to out of range 18S rRNA Cq.

8.7 kb mtDNA Deletion Preliminary Evaluation—Standard PCR

As discussed above in Example 3, we previously identified the 8.7 kb deletion using a combination of next generation sequencing (NGS) and proprietary data mining software in a set of 10 cases and 10 controls. As discussed above, we successfully detected the deletion in circulating plasma and performed further evaluation by qPCR to determine whether the target was detectable in rho 0 cells using more sensitive qPCR.

### Diagnostic Accuracy of the 8.7 kb Deletion

Having successfully detected the 8.7 kb deletion in circulating plasma and endometriosis lesions, we investigated whether the 8.7 kb deletion could discriminate between symptomatic controls versus all endometriosis; between the three subtypes; and between the revised American Society for Reproductive Medicine (r-ASRM) classification stages, in plasma from a larger set of clinical specimens. We performed the analyses using primarily the symptomatic controls and specimens from participants with confirmed disease to more accurately reflect the clinically relevant patient populations, that is, all presenting with symptoms of endometriosis. We also measured the frequency of the deletion in the asymptomatic control samples and did not detect a difference in the 8.7 kb deletion between symptomatic and asymptomatic (p=0.681) control specimens.

### Symptomatic Controls Versus all Disease

We were able to discriminate well between symptomatic control and all endometriosis specimens using the 8.7 kb assay. The AUC (95% CI) of 0.8007 (0.7035-0.8979) was statistically significant (p<0.0001). We examined ROC coordinates and chose a threshold to optimize sensitivity, with a threshold of 6.650 discriminating between symptomatic controls and all subtypes/stages of endometriosis and gave acceptable sensitivity and specificity values (Table 17).

TABLE 17
Performance of the 8.7 kb deletion at 65% specificity, cut-off 6.65

| | AUC [95% CI] | | Sensitivity (%) [95% CI] | |
|---|---|---|---|---|
| All disease | 0.8007 | [0.7035-0.8979] | 80.95 | [73.85-86.48] |
| Peritoneal | 0.8882 | [0.8043-0.9722] | 94.00 | [83.78-98.36] |
| Ovarian | 0.7766 | [0.6572-0.8960] | 76.60 | [62.78-86.40] |
| Deep infiltrating | 0.7359 | [0.6057-0.8661] | 72.00 | [58.33-82.53] |
| Stage I/II | 0.8361 | [0.7426-0.9295] | 86.52 | [77.00-92.12] |
| Stage III/IV | 0.7465 | [0.6232-0.8697] | 72.41 | [59.80-82.25] |

### Detection of Disease by Subtype

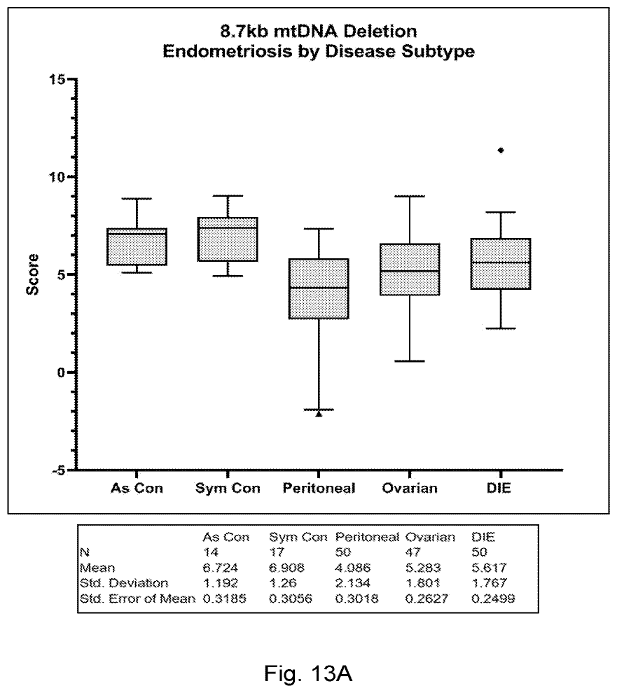

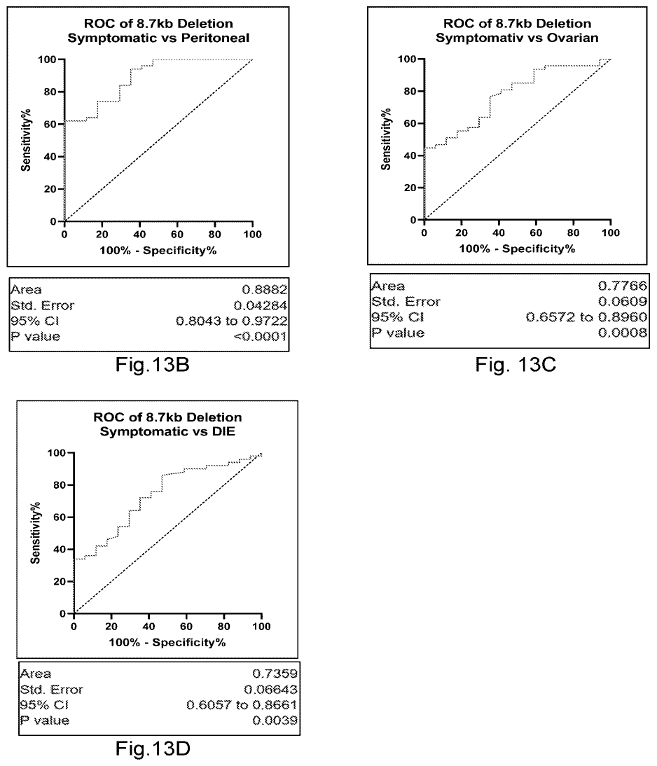

It is important that we can accurately detect all disease subtypes of endometriosis. In our study, the 8.7 kb deletion assay differentiated between specimens from symptomatic controls and those from patients with peritoneal, ovarian, and DI endometriosis (FIGS. 13A to 13D), with mean (SD) ΔCt values of 6.724 (1.192) for asymptomatic controls, 6.908 (1.26) for symptomatic controls, 4.086 (2.134) for peritoneal disease, 5.283 (1.801) for ovarian disease, and 5.617 (1.767) for DI endometriosis. Furthermore, the difference in normalized 8.7 kb deletion quantity between symptomatic controls was statistically significant for peritoneal (p<0.0001), ovarian (p=0.0002), and DI endometriosis (p=0.0023).

Diagnostic accuracy of the 8.7 kb deletion assay was also evaluated for each sub-type (FIGS. 13A to 13D). We accurately distinguished specimens from symptomatic controls and disease subtypes: AUC (95% CI) was 0.8882 (0.8043-0.9722; p<0.0001) for detection of peritoneal disease, 0.7766 (0.6572 0.8960; p=0.0008) for ovarian, and 0.7359 (0.6057-0.8661; p=0.0039) for DI endometriosis. In addition, the threshold value of 6.65 gave acceptable sensitivity and specificity values for distinguishing between symptomatic controls versus peritoneal endometriosis, ovarian, and DI disease (Table 17).

Detection of Disease by r-ASRM Stage

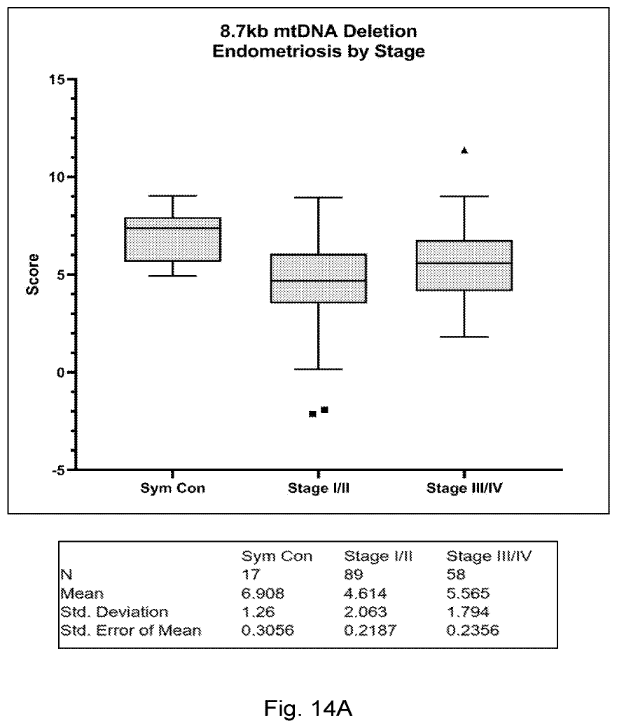

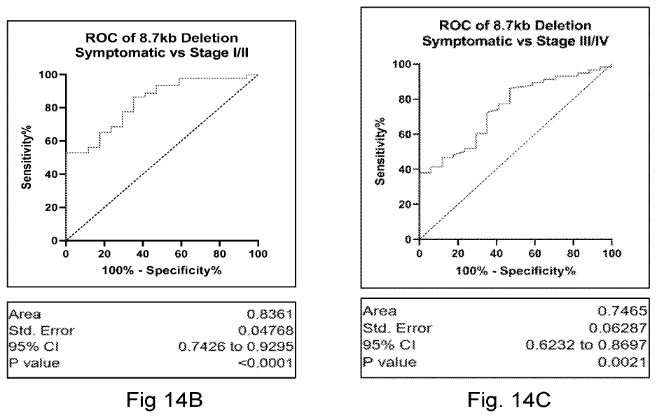

Endometriosis cases were classified into two stage groups, r-ASRM Stage I/II and Stage III/IV to determine if both low and high stage disease would be accurately identified using the 8.7 kb deletion. The 8.7 kb deletion assay differentiated between specimens from symptomatic controls and those from patients with low stage (Stage I/II) and high stage (Stage III/IV) (FIGS. 14A-14C), with mean (SD) ΔCt values of 6.908 (1.26) for symptomatic controls, 4.614 (2.063) for low stage and 5.565 (1.794) for high stage disease.

Diagnostic accuracy assessed by receiver operating curve was highest for Stage I/II: AUC 0.8361 (0.7426-0.9295; p<0.0001) compared to Stage II/IV: AUC 0.7465 (0.6232-0.8697; p=0.0021). At the threshold of 6.65 sensitivity and specificity for all stages was acceptable (Table 17).

Correlation with Patient Age, Specimen Age, Hormonal Therapy, and Menstrual Phase

For an ideal assay, diagnostic accuracy would not be affected by factors such as patient and specimen age, hormonal therapy, and menstrual phase. We found no correlation between the ΔCt values and patient age (p=0.749) nor specimen age by year of collection (p=0.222) (Table 3). Similarly, no statistically significant differences in ΔCt values were seen when we stratified participants by hormonal status (p=0.838) or menstrual phase (p=0.233) (Table 18).

TABLE 18
Effect of patient and specimen age, hormonal status and menstrual cycle

| | Spearman | | Kruskal-Wall test or Mann Whitney |
|---|---|---|---|
| Parameter | correlation (r) | p-value | U-test p-value |
| Patient age | 0.016 | 0.749 | — |
| Specimen age | 0.092 | 0.222 | — |
| Hormonal | — | — | 0.838 |
| status | | | |
| Menstrual | — | — | 0.233 |
| cycle | | | |

The Mann-Whitney U-Test was used to determine effect of hormonal status on detection of endometriosis. The Kruskal Wallis was used to determine effect of menstrual cycle on detection of endometriosis.

### Evaluating the Disease Specificity of the 8.7 kb Deletion for Endometriosis

To further assess whether other female diseases had elevated levels of the 8.7 kb deletion we obtained plasma samples from women who were subsequently diagnosed with endometrial cancer (n=12), ovarian cancer (n=72), and breast cancer (n=51) and compared the marker frequency to that of the three endometriosis subtypes (peritoneal, ovarian, and deep infiltrating endometriosis) as well as the symptomatic control group (FIGS. 14A-14C). Significantly less 8.7 kb deletion was detected in all three cancers with endometrial cancer estimated as having 64-fold less deletion than endometriosis, ovarian cancer 16-fold less, and breast cancer 8-fold less (p<0.0001). The results from this evaluation are provided in Table 19.

TABLE 19
Disease specificity of the 8.7 kb deletion for endometriosis

| | Endometrial | Ovarian | Breast | Symptomatic | | | |
|---|---|---|---|---|---|---|---|
| | Cancer | Cancer | Cancer | Controls | Peritoneal | Ovarian | DIE |
| N | 12 | 72 | 51 | 17 | 53 | 48 | 50 |
| Mean | 12.71 | 8.921 | 8.218 | 6.908 | 4.265 | 5.304 | 5.617 |
| Std. | 2.515 | 4.239 | 3.953 | 1.26 | 2.202 | 1.788 | 1.767 |
| Deviation | | | | | | | |
| Std. Error | 0.7261 | 0.4996 | 0.5536 | 0.3056 | 0.3024 | 0.258 | 0.2499 |
| of Mean | | | | | | | |

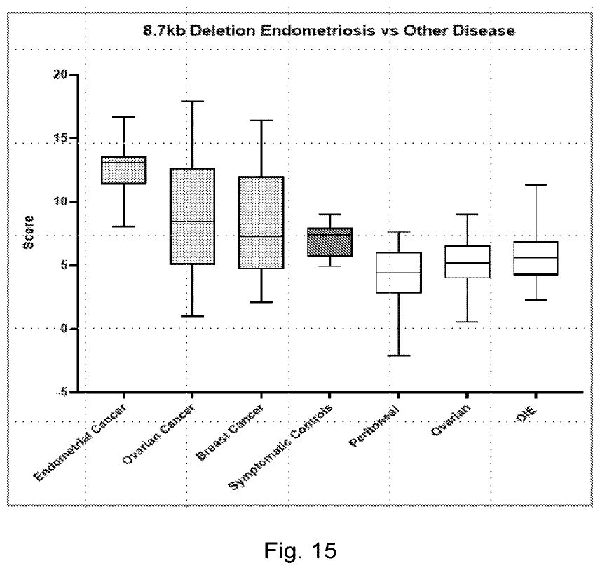

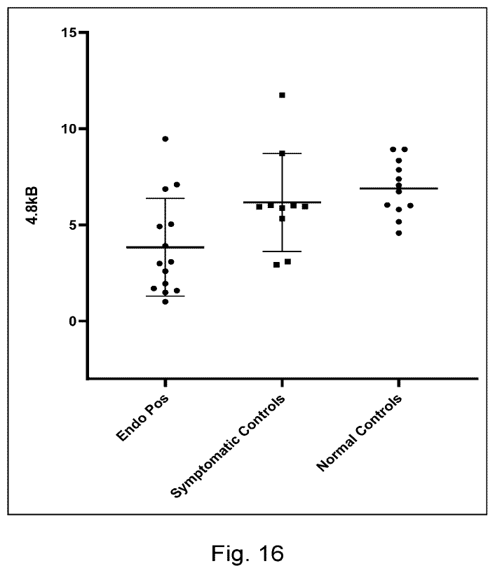

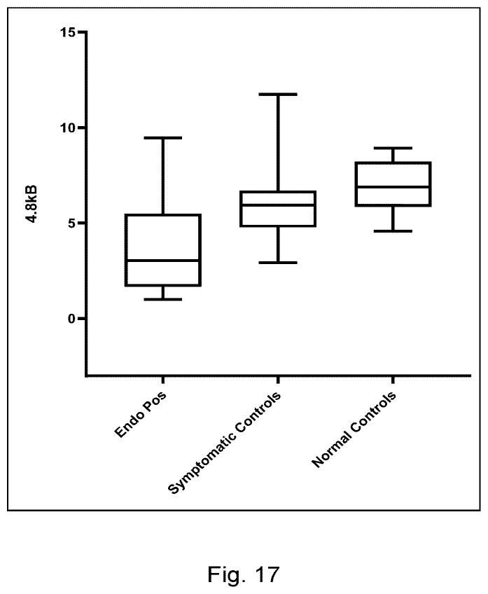

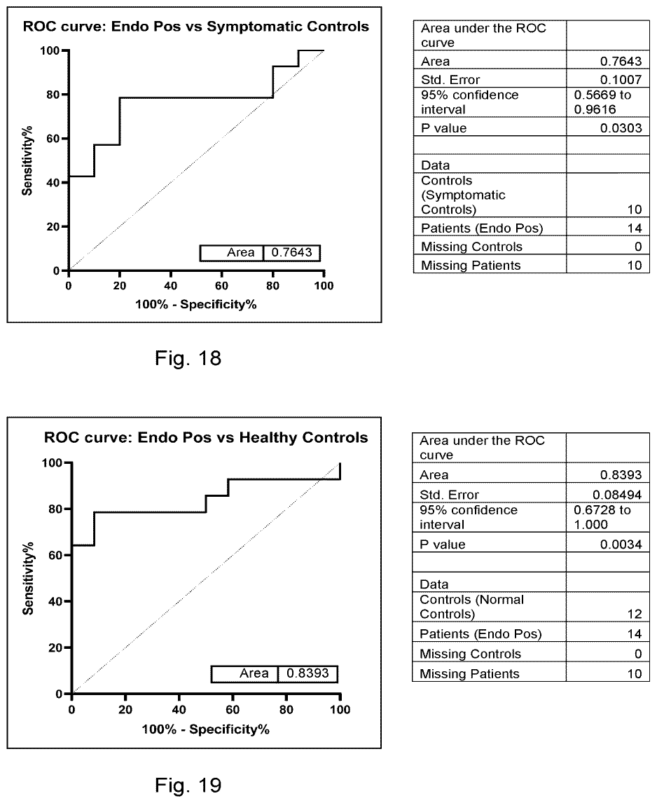

The data from Table 19 is illustrated in FIG. 15, which shows normalized 8.7 kb deletion distribution for specimens from endometrial cancer, ovarian cancer, breast cancer, symptomatic controls, and participants with peritoneal, ovarian or deep infiltrating endometriosis.

### Discussion

In the present study, we demonstrated the utility of measuring the levels of the 8.7 kb deletion biomarker in plasma samples as a potential assay for detecting endometriosis. Our assay met robustness criteria for diagnostic tests, utilized a minimally invasive specimen from blood, and accurately detected all subtypes and stages of disease, with best performance seen in the peritoneal sub-type and low stages of endometriosis, both encountered at high frequency in a primary care setting. When translated into clinical use, this assay could potentially shorten the time to diagnosis and enable medical intervention before surgery. Specimens are easy and inexpensive to collect via venipuncture, with a low possibility of co morbidities associated with this type of collection.

With good diagnostic accuracy, especially for low stage and peritoneal disease, the 8.7 kb deletion assay has the potential to augment the current standard of care, particularly in the diagnosis of peritoneal disease, which, unlike ovarian and deep infiltrating endometriosis, cannot be reliably detected by imaging modalities. The relative simplicity of the 8.7 kb deletion assay means it could be viable for use in both primary and secondary care settings. Blood samples are routinely collected in primary care without the need for dedicated surgical space or equipment. The high copy number of mtDNA means standard DNA extraction methods can be used without enrichment techniques. Furthermore, a high failure rate for the test is unlikely given the ample amount of DNA recovered from a standard blood specimen. Real time PCR based technology is widely used in clinical laboratories producing easily interpreted and quantitative results.

In our study we showed an absence of correlation between the deletion and patient age, specimen age, hormone status, or phase of menstrual cycle. A lack of correlation with menstrual stage simplifies sampling requirements, with no need to consider menstruation phase when scheduling sample collection.

The current complex diagnostic process, combined with symptoms that overlap with other gynecological disorders, mean the epidemiology of endometriosis has not been fully characterized, yet this is a key element in the successful management of any condition [1, 2, 4, 6]. The advent of molecular assays and novel biomarkers will provide additional, more readily available data to help improve our understanding of endometriosis epidemiology. Importantly, our study used publicly available and standardized methods for collecting and processing specimens, which allows a more direct comparison of test results across studies and patient populations [41-44, 53].

### Conclusion

The assay described above, using the mitochondrial derived 8.7 kb deletion biomarker, is a minimally invasive, blood sample based method for diagnosing endometriosis that may be used in both primary and secondary clinical settings. The relatively simple and more patient friendly approach provided by this assay would shorten the time to diagnosis and thereby improve the management of the debilitating condition described herein and thereby improve patients' quality of life.

### Example 5: Identification of 4.8 kb mtDNA Deletion for Detecting Endometriosis

A study similar to that described above was conducted for identifying a correlation between the frequency of the 4.8 kb mtDNA deletion (Deletion ID 8590) and endometriosis. The method of Example 4 was followed for this analysis. The primers used for this study are shown in Table 20.

TABLE 20
Primer sequences used for amplification of 4.8 kb deletion

| | Primer | | | |
|---|---|---|---|---|
| Deletion ID | Direction | Length | Sequence | SEQ ID NO. |
| 8590 | Fwd | 21 | TGCCCTAGCCCACTTCTTACC | 80 |
| (“4.8 kb | Rev | 25 | TAGTTGAGGTCTAGGGCTGTTGGTT | 81 |
| Deletion”) | (junction | | | |
| | primer) | | | |

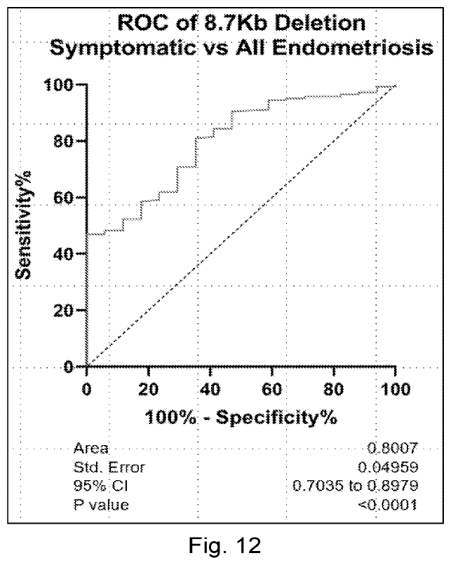

Data illustrating the utility of the 4.8 kb deletion in detecting endometriosis is provided in Table 21 and illustrated in FIGS. 12-14.

TABLE 21
Identification of 4.8 kb mtDNA deletion

| | | Symptomatic | Normal Healthy |
|---|---|---|---|
| | Endo Positive | Controls | Controls |
| Number of values | 14 | 10 | 12 |
| Minimum | 1.005 | 2.93 | 4.575 |
| 25% Percentile | 1.67 | 4.771 | 5.85 |
| Median | 3.033 | 5.945 | 6.89 |
| 75% Percentile | 5.489 | 6.7 | 8.224 |
| Maximum | 9.47 | 11.74 | 8.93 |
| Range | 8.465 | 8.81 | 4.355 |
| Mean | 3.833 | 6.161 | 6.899 |
| Std. Deviation | 2.537 | 2.547 | 1.436 |
| Std. Error of Mean | 0.6781 | 0.8053 | 0.4147 |
| Lower 95% CI of mean | 2.368 | 4.339 | 5.986 |
| Upper 95% CI of mean | 5.298 | 7.982 | 7.811 |
| Sum | 53.67 | 61.61 | 82.79 |

Although the above description includes reference to certain specific embodiments, various modifications thereof will be apparent to those skilled in the art. Any examples provided herein are included solely for the purpose of illustration and are not intended to be limiting in any way. Any drawings provided herein are solely for the purpose of illustrating various aspects of the description and are not intended to be drawn to scale or to be limiting in any way. The scope of the claims appended hereto should not be limited by the preferred embodiments set forth in the above description but should be given the broadest interpretation consistent with the present specification as a whole. The disclosures of all documents recited herein are incorporated herein by reference in their entirety.

### REFERENCE

1. Eskenazi B, Warner M L. Epidemiology of endometriosis. Obstet. Gynecol. Clin. North Am. 24(2), 235-258 (1997).
2. Kjerulff K H, Erickson B A, Langenberg P W. Chronic gynecological conditions reported by US women: findings from the National Health Interview Survey, 1984 to 1992. Am J Public Health. 86(2), 195-199 (1996).
3. Houston D E, Noller K L, Melton L J, Selwyn B J, Hardy R J. Incidence of pelvic endometriosis in Rochester, Minnesota, 1970-1979. Am. J. Epidemiol. 125(6), 959-969 (1987).
4. Wheeler JM. Epidemiology of endometriosis-associated infertility. J Reprod Med. 34(1), 41-46 (1989).
5. Giudice L C, Kao L C. Endometriosis. Lancet. 364(9447), 1789-1799 (2004).
6. Vessey M P, Villard-Mackintosh L, Painter R. Epidemiology of endometriosis in women attending family planning clinics. BMJ. 306(6871), 182-184 (1993).
7. Zondervan K T, Becker C M, Koga K, Missmer S A, Taylor R N, Viganò P. Endometriosis. Nature Reviews Disease Primers. 4(1), 9 (2018).
8. Barlow D H, Kennedy S. Endometriosis: new genetic approaches and therapy. Annu. Rev. Med. 56, 345-356 (2005).
9. Giudice LC. Clinical practice. Endometriosis. N. Engl. J. Med. 362(25), 2389-2398 (2010).
10. Fassbender A, Vodolazkaia A, Saunders P, et al. Biomarkers of endometriosis. Fertil. Steril. 99(4), 1135-1145 (2013). **Review on recent work in biomarkers of endometriosis
11. S K, Kantawala K P, Menias C O. Beyond the boundaries-endometriosis: typical and atypical locations. Curr Probl Diagn Radiol. 40(6), 219-232 (2011).
12. Berkley K J, Rapkin A J, Papka R E. The pains of endometriosis. Science. 308(5728), 1587-1589 (2005).
13. Nnoaham K E, Hummelshoj L, Webster P, et al. Impact of endometriosis on quality of life and work productivity: a multicenter study across ten countries. Fertil. Steril. 96(2), 366-373.e8 (2011).
14. Simoens S, Dunselman G, Dirksen C, et al. The burden of endometriosis: costs and quality of life of women with endometriosis and treated in referral centres. Hum. Reprod. 27(5), 1292-1299 (2012).
15. Dunselman G a. J, Vermeulen N, Becker C, et al. ESHRE guideline: management of women with endometriosis. Hum. Reprod. 29(3), 400-412 (2014).
16. Hadfield R, Mardon H, Barlow D, Kennedy S. Delay in the diagnosis of endometriosis: a survey of women from the USA and the UK. Hum. Reprod. 11(4), 878-880 (1996).
17. Ahn S H, Singh V, Tayade C. Biomarkers in endometriosis: challenges and opportunities. Fertil. Steril. 107(3), 523-532 (2017). **Review on recent work in biomarkers of endometriosis
18. Rifai N, Gillette M A, Carr S A. Protein biomarker discovery and validation: the long and uncertain path to clinical utility. Nat. Biotechnol. 24(8), 971-983 (2006).
19. Hu C, van der Heijden R, Wang M, van der Greef J, Hankemeier T, Xu G. Analytical strategies in lipidomics and applications in disease biomarker discovery. J. Chromatogr. B Analyt. Technol. Biomed. Life Sci. 877(26), 2836-2846 (2009).
20. Liotta L A, Petricoin E F. Serum peptidome for cancer detection: spinning biologic trash into diagnostic gold. J. Clin. Invest. 116(1), 26-30 (2006).
21. Petricoin E F, Ardekani A M, Hitt B A, et al. Use of proteomic patterns in serum to identify ovarian cancer. Lancet. 359(9306), 572-577 (2002).
22. Petricoin E F, Belluco C, Araujo R P, Liotta L A. The blood peptidome: a higher dimension of information content for cancer biomarker discovery. Nat. Rev. Cancer. 6(12), 961-967 (2006).
23. Zhang Z, Bast R C, Yu Y, et al. Three biomarkers identified from serum proteomic analysis for the detection of early stage ovarian cancer. Cancer Res. 64(16), 5882-5890 (2004).
24. Anderson N L, Anderson N G. The human plasma proteome: history, character, and diagnostic prospects. Mol. Cell Proteomics. 1(11), 845-867 (2002).
25. May K E, Conduit-Hulbert S A, Villar J, Kirtiey S, Kennedy S H, Becker C M. Peripheral biomarkers of endometriosis: a systematic review. Hum. Reprod. Update. 16(6), 651-674(2010).
26. Rahmioglu N, Fassbender A, Vitonis A F, et al. Harmonization of Clinical and Laboratory Data to Improve Biomarker Discovery in Endometriosis: WERF EPHect. In: Biomarkers for Endometriosis. Springer, Cham, 199-226. ** Summary of standardized methods in endometriosis research
27. Taylor R W, Tumbull D M. Mitochondrial DNA mutations in human disease. Nat. Rev. Genet. 6(5), 389-402 (2005).
28. Polyak K, Li Y, Zhu H, et al. Somatic mutations of the mitochondrial genome in human colorectal tumours. Nat. Genet. 20(3), 291-293 (1998).
29. Petros J A, Baumann A K, Ruiz-Pesini E, et al. mtDNA mutations increase tumorigenicity in prostate cancer. Proc. Natl. Acad. Sci. U.S.A. 102(3), 719-724 (2005).
30. Creed J, Klotz L, Harbottle A, et al. A single mitochondrial DNA deletion accurately detects significant prostate cancer in men in the PSA “grey zone.” World J Urol. 36(3), 341-348 (2018).
31. Parrella P, Xiao Y, Fliss M, et al. Detection of mitochondrial DNA mutations in primary breast cancer and fine-needle aspirates. Cancer Res. 61(20), 7623-7626 (2001).
32. Liang B C. Evidence for association of mitochondrial DNA sequence amplification and nuclear localization in human low-grade gliomas. Mutat. Res. 354(1), 27-33 (1996).
33. Guo X-G, Guo Q-N. Mutations in the mitochondrial DNA D-Loop region occur frequently in human osteosarcoma. Cancer Lett. 239(1), 151-155 (2006).
34. Alonso A, Martin P, Albarran C, et al. Detection of somatic mutations in the mitochondrial DNA control region of colorectal and gastric tumors by heteroduplex and single-strand conformation analysis. Electrophoresis. 18(5), 682-685 (1997).
35. Liu V W, Shi H H, Cheung A N, et al. High incidence of somatic mitochondrial DNA mutations in human ovarian carcinomas. Cancer Res. 61(16), 5998-6001 (2001).
36. Dai J G, Xiao Y B, Min J X, Zhang G Q, Yao K, Zhou R J. Mitochondrial DNA 4977 BP deletion mutations in lung carcinoma. Indian J Cancer. 43(1), 20-25 (2006).
37. Liu V W S, Yang H J, Wang Y, et al. High frequency of mitochondrial genome instability in human endometrial carcinomas. Br. J. Cancer. 89(4), 697-701 (2003).
38. Wang Y, Xue W C, Liu V W S, Ngan H Y S. Detection of mosaic pattern of mitochondrial DNA alterations in different populations of cells from the same endometrial tumor. Mitochondrion. 7(1-2), 171-175 (2007).
39. Semczuk A, Lorenc A, Putowski L, et al. Clinicoprognostical features of endometrial cancer patients with somatic mtDNA mutations. Oncol. Rep. 16(5), 1041-1045 (2006).
40. Pejovic T, Ladner D, Intengan M, et al. Somatic D-loop mitochondrial DNA mutations are frequent in uterine serous carcinoma. Eur. J. Cancer. 40(16), 2519-2524 (2004).
41. Rahmioglu N, Fassbender A, Vitonis A F, et al. World Endometriosis Research Foundation Endometriosis Phenome and Biobanking Harmonization Project: III. Fluid biospecimen collection, processing, and storage in endometriosis research. Fertil. Steril. 102(5), 1233-1243 (2014). *Harmonized methods for endometriosis research
42. Fassbender A, Rahmioglu N, Vitonis A F, et al. World Endometriosis Research Foundation Endometriosis Phenome and Biobanking Harmonisation Project: IV. Tissue collection, processing, and storage in endometriosis research. Fertil. Steril. 102(5), 1244-1253 (2014). *Harmonized methods for endometriosis research
43. Becker C M, Laufer M R, Stratton P, et al. World Endometriosis Research Foundation Endometriosis Phenome and Biobanking Harmonisation Project: I. Surgical phenotype data collection in endometriosis research. Fertil. Steril. 102(5), 1213-1222 (2014). *Harmonized methods for endometriosis research
44. Vitonis A F, Vincent K, Rahmioglu N, et al. World Endometriosis Research Foundation Endometriosis Phenome and Biobanking Harmonization Project: II. Clinical and covariate phenotype data collection in endometriosis research. Fertil. Steril. 102(5), 1223-1232 (2014). *Harmonized methods for endometriosis research
45. American Society for Reproductive Medicine. Revised American Society for Reproductive Medicine classification of endometriosis: 1996. Fertility and Sterility. 67(5), 817-821.
46. King M P, Attardi G. Human cells lacking mtDNA: repopulation with exogenous mitochondria by complementation. Science. 246(4929), 500-503 (1989).
47. Brandon M C, Lott M T, Nguyen K C, et al. MITOMAP: a human mitochondrial genome database—2004 update. Nucleic Acids Res. 33(Database issue), D611-613 (2005).
48. Stratton P, Winkel C, Premkumar A, et al. Diagnostic accuracy of laparoscopy, magnetic resonance imaging, and histopathologic examination for the detection of endometriosis. Fertil. Steril. 79(5), 1078-1085 (2003).
49. Mettler L, Schollmeyer T, Lehmann-Willenbrock E, et al. Accuracy of laparoscopic diagnosis of endometriosis. JSLS. 7(1), 15-18 (2003). *Evaluation of current gold standard for diagnosis of endometriosis
50. Falcone T, Mascha E. The elusive diagnostic test for endometriosis. Fertil. Steril. 80(4), 886-888 (2003).
51. Wykes C B, Clark T J, Khan K S. Accuracy of laparoscopy in the diagnosis of endometriosis: a systematic quantitative review. BJOG. 111(11), 1204-1212 (2004).
52. Harel Z. Dysmenorrhea in adolescents and young adults: etiology and management. J Pediatr Adolesc Gynecol. 19(6), 363-371 (2006).
53. Casper R F. Introduction: new tools for enhancing collaborative endometriosis research. Fertil. Steril. 102(5), 1211-1212 (2014).
54. Salahi, E., et al.; Mitochondrial DNA 4977-bp deletion in endometriosis. Genes & Genomics, 35(5), 563-567 (2013).
55. Taylor H S. Diagnosing endometriosis: Is laparoscopy the gold standard? Supplement to OBG Management. S1-S2 (2019).
56. Ballard K, Lowton K, Wright J. What's the delay? A qualitative study of women's experiences of reaching a diagnosis of endometriosis. Fertil. Steril 86(5), 1296-301 (2006).
57. Creed J, Maggrah A, Reguly B, Harbottie A. Mitochondrial DNA deletions accurately detect endometriosis in symptomatic females of child-bearing age. Biomark. Med. 13(4), 291-306 (2019).
58. The Practice Committee of the American Society for Reproductive Medicine. Endometriosis and infertility. Fertil. Steril. 81, 1441-1446 (2004).
59. Nicholls, T. J., et al., Linear mtDNA fragments and unusual mtDNA rearrangements associated with pathological deficiency of MGME1 exonuclease. Hum Mol Genet., 23(23):6147-62 (2014).
60. Zhang C. et al., Multiple mitochondrial DNA deletions in an elderly human individual. FEBS, 297, 1, 2, 34-38 (1992).
61. Wei, Y. H., Mitochondrial DNA alterations as ageing-associated molecular events. Mutat. Res., 275(3-6), 145-55 (1992).
62. Tanaka, M. et al., Direct sequencing of deleted mitochondrial DNA in myopathic patients. Biochem Biophys Res Commun., 164(1):156-63, (1989).
63. Kapsa, R. et al., A novel mtDNA deletion in an infant with Pearson syndrome. J Inherit Metab Dis., 17(5):521-6, (1974).
64. Hseih, R. H., et al., Multiple rearrangements of mitochondrial DNA in unfertilized human oocytes. Fertil Steril., 77(5):1012-7, (2002).
65. Hjelm, B. E. et al., http://mitobreak.portugene.com/cgi-bin/Mitobreak_individual_page.cgi?Name=5371:14058&Breaktype=Del&Species=Homsap&Published=Pub

1-54: (canceled)

55. A method of identifying, in a biological sample from a mammalian subject, an aberrant mitochondrial DNA, mtDNA, molecule having a deletion, wherein once re-circularized, the mtDNA includes a junction point consisting of first and second nucleotides, and wherein, with respect to SEQ ID NO: 1:
the deletion includes nucleotides 5377-14048, the first nucleotide is between nucleotides 5362-5377 and the second nucleotide is between nucleotides 14048-14063.

56. The method of claim 55, wherein:
the deletion includes nucleotides 5377-14048, the first nucleotide is at position 5362 and the second nucleotide is at position 14049.

57. The method of claim 55, wherein the aberrant mtDNA comprises the nucleotide sequence set forth in SEQ ID NO: 75.

58. The method of claim 55, wherein the identification comprises contacting the biological sample with:
a) a nucleic acid probe having a nucleotide sequence substantially complementary to a portion of the nucleotide sequence of the aberrant mtDNA comprising the junction point;
b) a nucleic acid primer pair, wherein one of the primers has a nucleotide sequence complementary to a portion of the nucleotide sequence of the aberrant mtDNA comprising the junction point; or
c) a nucleic acid primer pair, wherein each of the primers has a nucleotide sequence complementary to nucleotide sequences of the aberrant mtDNA adjacent to the junction point.

59. The method of claim 58, wherein the one of the primers of the pair of primers has the nucleotide sequence set forth in SEQ ID NO: 83.

60. The method of claim 58, wherein the other primer of the pair of primers has the nucleotide sequence set forth in SEQ ID NO: 82.

61. A method of identifying, in a biological sample from a mammalian subject, a fusion transcript encoded by:
a) at least a portion of an aberrant mitochondrial DNA, mtDNA, molecule having a deletion resulting in a junction point after the mtDNA is re-circularized, wherein the junction point is between nucleotides 5362:14049 of the mtDNA nucleotide sequence of SEQ ID NO: 1; or
b) at least a portion of an aberrant mitochondrial DNA, mtDNA, molecule as defined in claim 55.

62. The method of claim 61, wherein the method comprises:
a) contacting the biological sample with a nucleic acid probe having a nucleic acid sequence that is complementary to the nucleotide sequence of the fusion transcript having the transcribed junction point; or
b) identifying a translation product of the fusion transcript.

63. The method of claim 62, wherein:
the fusion transcript comprises the nucleotide sequence set forth in SEQ ID NO: 77; or
the translation product has the amino acid sequence set forth in SEQ ID NO: 79.

64. The method of claim 55, wherein the method is for detecting, diagnosing, and/or monitoring endometriosis in the mammalian subject.

65. The method of claim 61, wherein the method is for detecting, diagnosing, and/or monitoring endometriosis in the mammalian subject.

66. The method of claim 55, further comprising quantifying, in the biological sample, the amount of the aberrant mtDNA and comparing the quantified amount of aberrant mtDNA to a reference value indicative of the presence of endometriosis or the development of endometriosis in the subject.

67. The method of claim 61, further comprising quantifying, in the biological sample, the amount of the aberrant mtDNA and comparing the quantified amount of aberrant mtDNA to a reference value indicative of the presence of endometriosis or the development of endometriosis in the subject.

68. The method of claim 55, wherein the biological sample is one or more of: blood; a blood derivative, such as plasma and/or serum; tissue; and menstrual fluid.

69. The method of claim 61, wherein the biological sample is one or more of: blood; a blood derivative, such as plasma and/or serum; tissue; and menstrual fluid.

70. A method of identifying, in a biological sample from a mammalian subject, a deleted mitochondrial DNA, mtDNA, molecule, wherein the deletion comprises nucleotides 5362-14049; 8469-13447; 7992-15730; 9191-12909; 9188-12906; 10367-12829; 6260-12814; 7973-9023; 9086-10313; 9079-14988; 7260-15540; 8431-10841; or 8984-13833 of the mtDNA nucleotide sequence of SEQ ID NO: 1.

71. The method of claim 70, wherein the method is for detecting, diagnosing, and/or monitoring endometriosis in the mammalian subject.

72. (canceled)

73. A kit for conducting the method according to claim 60, wherein the kit comprises at least one of:
a) a nucleic acid primer pair, wherein one of the primers has a nucleotide sequence complementary to a portion of the nucleotide sequence of the aberrant mtDNA comprising the junction point; or
b) a nucleic acid primer pair, wherein each of the primers has a nucleotide sequence complementary to nucleotide sequences of the aberrant mtDNA adjacent to the junction point.

74. A kit for conducting the method according to claim 62, wherein the kit comprises at least one of:
a) primers and/or probes complementary to one or more fusion transcripts of the aberrant mtDNA molecules; or
b) binding agents, such as antibodies or antibody fragments, that are adapted to bind to proteins encoded by the aberrant mtDNA molecules.

75. The method of claim 61, wherein the portion includes the junction point.

76-81. (canceled)
